(19) United States
(12) Patent Application Publication
(10) Pub. No.: US 2019/0025297 A1
(43) Pub. Date: Jan. 24, 2019

(54) Stepwise and Blockwise Biochemical Network Laboratory Breadboard Systems and Techniques for Signaling, Disease Research, Drug Discovery, Cell Biology, and Other Applications

(71) Applicant: NRI R&D PATENT LICENSING, LLC, San Antonio, TX (US)

(72) Inventors: Lester F. Ludwig, San Antonio, TX (US); Pooncharas Tipgunlakant, San Francisco, CA (US); Hong Ye Wu, San Francisco, CA (US); Catherine Luk, San Francisco, CA (US)

(21) Appl. No.: 14/485,791

(22) Filed: Sep. 14, 2014

Related U.S. Application Data
(63) Continuation-in-part of application No. 14/216,420, filed on Mar. 17, 2014.
(60) Provisional application No. 61/802,127, filed on Mar. 15, 2013.

(51) Int. Cl. G01N 33/543; B01L 3/00; G05D 7/06; G06F 19/12; G06F 17/18; G06F 19/24; G06F 19/28; C12M 1/36; G01N 35/10

(52) U.S. Cl. CPC G01N 33/54373; B01L 3/502715; G05D 7/0694; G06F 19/12; G06F 17/18; B01L 2300/0816; G06F 19/28; C12M 41/48; G01N 35/1095; B01L 2200/028; B01L 2200/027; G06F 19/24

(57) ABSTRACT

A “breadboard” approach by which a biochemical pathway under study is separated or segmented into smaller portions, at least one of which can to a degree of approximation be accurately emulated with a replica microscale and/or nanoscale fluidic implementation whose constituent species, inhibitor(s), catalyst(s) and other reaction agent(s) can be closely controlled and at least one aspect of whose behavior can be measured. Control and measurement information interfaces with a computer that executes algorithms comprising one or more of a control process, control event-script, experiment, data recording, and mathematical model. A model can be used to simulate the actions, behavior, or other aspects of another portion of the biochemical signaling process, pathway, or network. Replica constituents can include enzymes, other proteins, lipids, ions, peptides, and other materials provided under controlled conditions and timing, as well as varying degrees of competitive species, drugs, environmental influences, and substitute or representative molecular crowding.

### CROSS-REFERENCE TO RELATED APPLICATIONS

This application is a Continuation-In-Part of pending U.S. patent application Ser. No. 14/216,420, filed Mar. 17, 2014, which claims benefit of priority from Provisional U.S. Patent application Ser. No. 61/802,127, filed Mar. 15, 2013, the contents of which are incorporated by reference.

### COPYRIGHT & TRADEMARK NOTICES

A portion of the disclosure of this patent document may contain material, which is subject to copyright protection. Certain marks referenced herein may be common law or registered trademarks of the applicant, the assignee or third parties affiliated or unaffiliated with the applicant or the assignee. Use of these marks is for providing an enabling disclosure by way of example and shall not be construed to exclusively limit the scope of the disclosed subject matter to material associated with such marks.

### BACKGROUND

### Field of the Invention

The present disclosure pertains to next generation tools for the study of biological signaling processes and networks in living biological cells, and in particular to laboratory tools, methods, microscale sensors, microscale instrumentation, microfluidic, computerized instrumentation, computer simulation, and computer analysis tools relating to the study, analysis, and modeling of biological cell signaling. Aspects of the present application can also be readily used or adapted for the study, analysis, and modeling of biochemical processes and pathways for metabolism and gene regulation as well as laboratory or industrial activities pertaining to confined-environment chemistry, intercalation chemistry, chemical reactions in constrained systems, molecular encapsulation, and aspects of host-guest chemistry and the synthesis and study of meta-materials.

### Related Art

Material related to the topic of this patent application is provided in three earlier pending patent applications by one of the present inventors, specifically U.S. patent application Ser. No. 14/216,420, U.S. patent application Ser. No. 13/157,304, and paragraphs [00565] through [00569] of U.S. patent application Ser. No. 13/761,142. Pending patent application U.S. Ser. No. 13/761,142 additionally teaches throughout a variety of microscale sensors, microscale instrumentation, computer-controlled microfluidics, and computer-controlled instrumentation systems and methods useful to the present patent application. U.S. Pat. No. 8,594,848, also by the present inventor, teaches throughout additional computer-controlled microfluidics, microscale instrumentation, and computer-controlled instrumentation systems and methods useful to the present patent application. Allowed patent application U.S. Ser. No. 12/931,867, also by the present inventor, teaches throughout various microfluidic chemistry and further computer-controlled chemical processing systems and methods useful to the present patent application. U.S. Pat. Nos. 8,032,258 and 8,606,414 and pending U.S. patent application Ser. No. 13/251,288, also by the present inventor, teach controllable multichannel microfluidic chemical bus systems and methods useful to the present patent application.

Importance of Biochemical Signaling Pathways and Networks

Biochemical signaling networks play considerable roles in the cell cycle and most diseases, for example cancer. In addition to complicated multiple feedback loops and feedforward paths that regulate resulting dynamics, biochemical signaling networks include crosstalk among pathways, mechanical aspects of transport, conformation-dependent allosteric (state-dependent) reaction dynamics, enzyme recovery dynamics, exogenous regulatory controls, and many other exotic processes that are both critical to life processes and extremely complex. These render complex dynamics whose normal behavior, pathologies, and sensitivities are barely understood.

A more extensive analytical, quantitative, confirmative, and predictive understanding of biochemical signaling process, pathways, and networks is becoming increasingly indispensable. Comprehensive, accurate understanding and predictive modeling of biochemical signaling will play critical roles in future diagnostics and drug discovery, increasing replacing the roles of QSAR and other expensive and extensive approaches which, after a spectacular run of initial valuable productivity, have entirely failed to deliver new drugs. Further, the wide variability of side effects (both traceable and not traceable to the presence of undesirable enantiomers resultant from drug manufacturing processes) depends on the variability among patients' personal metabolic and signaling makeup. Additionally, many diseases such as cancer (lung cancer being a vibrant example) appear mechanistically to be optimally conquered via highly individualized analysis and therapies not unlike the notions of personalized or individualized medicine that are advocated for other sometimes controversial reasons. At the structural center of realistic approaches to all of these is again a comprehensive, accurate understanding and predictive modeling of biochemical signaling processes and signaling networks.

To date the approaches and results are at once both spectacular and primitive. A large number of signaling processes and pathways have been identified, with many new ones identified or conjectured every month. Many of these newly identified or conjectured signaling processes and pathways provide brand new understandings and explanations, and at times entirely unknown new phenomena. Experimental study of signaling processes and pathways have employed an impressive spectrum of technologies and methods but in many ways are limited to the introduction of instrumentation-observable markers into living cells, the testing of gross-effect biomarkers produced by living cells, laboratory-scale biochemical reaction studies, and probing of large molecule structure by biophysics and spectroscopic techniques. As powerful as these techniques have proven to be, they still suffer from immense limitation with regards to the types of behavior they can observe and characterize.

Examples of Current Biophysical Techniques Used to Study Protein-Protein Interactions

Some example techniques used to study protein-protein interactions include:
Co-Immunoprecipitation (Co-IP)
Pull-Down Assay
Crosslinking Protein Interaction Analysis
Label Transfer Protein Interaction Analysis
Far-Western Blot Analysis
Analytical Ultracentrifugation (AUC)
Flow cytometry
Fluorescence Spectroscopy
Surface Plasmon Resonance
Calorimetry

These are touched upon briefly in the subsections to follow.

Co-Immunoprecipitation (Co-IP)

Immunoprecipitation of intact protein also known as co-immunoprecipitation (Co-IP) is able to select an antibody that targets a known protein from a larger complex of proteins. The whole protein complex can be taken out which allows identification of unknown members of the complex. The proteins involved in the complex have to bind to each other tightly locking one member of the complex with an antibody. The concept of pulling protein complexes out of solution can also be referred to as “pull-down.” An example of the use of Co-IP in the study of the Hedgehog signaling pathway is provided in C. Tong; J. Jiang, “Using Immunoprecipitation to Study Protein—Protein Interactions in the Hedgehog-Signaling Pathway”. Methods In Molecular Biology Vol. 397, 2007, pp. 215-229 (available at http://link.springer.com/protocol/10.1007/978-1-59745-516-9_15).

Pull-Down Assay

Pull-down assay is a type of immunoprecipitation except that is the precipitation of target proteins instead of antibodies. It is an in vitro method capable of determining the physical interactions between proteins. This technique is usually used to confirm the existence of protein-protein interactions anticipated by other technique such as Co-IP but also to determine the existence of unknown protein-protein interactions. An example of the use of pull-down assay in the study of the Ras-Raf-MEK-ERK signaling pathway is provided in C. Song; W. Wang; M. Li; Y. Liu; D. Zheng, “Tax1 enhances cancer cell proliferation via Ras-Raf-MEK-ERK signaling pathway,” IUBMB Life. 2009 June; 61(6): pp. 685-92 (available at http://www.ncbi.nlm.nih.gov/pubmed/19472191).

Crosslinking Protein Interaction Analysis

Crosslinking reagents covalently bind protein-protein complexes together as they interact giving a method to measure them. Generally protein-protein interactions happen in short period of time. Crosslinking protein interaction analysis is able to freeze these brief contacts to study the proteins involved and the way they interact. An example of the use of crosslinking protein interaction analysis in the study of the MAPK signaling pathway is provided in W-K. Weng; L. Jarvis; T. W. LeBien, “Signaling through CD19 Activates Vav/Mitogen-Activated Protein Kinase Pathway and Induces Formation of a CD19/Vav/Phosphatidylinositol 3-Kinase Complex in Human B Cell Precursors,” Journal of Biological Chemistry 269:32514, 1994 (available at http://web.stanford.edu/˜wkweng/home.med/jbc/jbc.text.htm)

Label Transfer Protein Interaction Analysis

Label transfer tags proteins interacting with a protein of interest. Its technique blends with crosslinking technique to study protein-protein interactions. This method is enabling to uncover new interactions, endorse interactions proposed by other methods, as well as investigating the interface of interacting proteins. The label transfer technique can also detect weak or transient protein interactions regularly bypassing detection in co-immunoprecipitation and pull-down methods. An example of the use of label transfer protein interaction analysis in the study of the MAPK signaling pathway is provided in S. S. Andrews; Z. B. Hill; D. J. Maly, “Label Transfer Reagents to Probe p38 MAPK Binding Partners”, Chembiochem. Jan. 21, 2013; 14(2), pp. 209-216 (available at http://www.ncbi.nlm.nih.gov/pmc/articles/PMC3762675/#!po=86.1111)

Far-Western Blot Analysis

Far-Western blotting is a molecular biological method based on the technique of Western blotting to detect in vitro protein-protein interaction. In general, Western blotting identifies the protein of interest by using an antibody, while Far-Western blotting uses a non-antibody protein capable of binding to the protein of interest. Therefore, Western blotting is more useful for the detection of certain proteins, and Far-Western blotting to detect protein-protein interactions. An example of the use of far-western blot analysis in the study of the nuclear factor-kappaB signaling pathway is provided in X. Hu; O. Nesic-Taylor; J. Qiu; H. C. Rea; R. Fabian; D. K. Rassin; J. R. Perez-Polo, “Activation of nuclear factor-kappaB signaling pathway by interleukin-1 after hypoxia/ischemia in neonatal rat hippocampus and cortex,” J. Neurochem, 2005 April; 93(1), pp. 26-37 (available at http://www.ncbi.nlm.nih.gov/pubmed/15773902).

Analytical Ultracentrifugation (AUC)

Analytical Ultracentrifugation (AUC) applies the principles of centrifugal acceleration to separate components of samples given the shape and mass differences. This technique requires a rotor capable of spinning samples at speeds enough to create forces that are up to tens of thousands times greater than that of gravity. AUC is capable of performing analysis of the concentration of the samples during centrifugation by implementing light detection devices into the system. Using ultraviolet light absorption and/or interference optical refractive index sensitive system allows the operator to observe the sample concentration versus the axis of rotation. AUC primarily performs two types of analysis: sedimentation velocity and sedimentation equilibrium. An example of the use of AUC in the study of the Notch signaling pathway is provided in A. G. Allgood; D. Barrick, “Mapping the Deltex-binding surface on the notch ankyrin domain using analytical ultracentrifugation,” J Mol Biol. 2011 Nov. 25; 414(2), pp. 243-59, (available at http://www.ncbi.nlm.nih.gov/pubmed/22001695).

Flow Cytometry

Flow Cytometry is a technique in which cells are suspended in a fluid flowing through a focus of exciting light that is scattered. Usually labeled with fluorescent markers, the light is first absorbed then emitted at changed frequencies. The scattered or emitted light is measured by a sensor that is able to detect the size and molecular characteristics of individual cells. Flow cytometry enables tens of thousands of cells to be examined per minute and gathers the data to be processed by computer. An example of the use of flow cytometry in the study of the MAPK signaling pathway is provided in A. Mavropoulos; D. P. Bogdanos; C. Liaskos; T. Orfanidou; T. Simopoulou; E. Zafiriou; L. I. Sakkas; E. I. Rigopoulou, “Flow Cytometric Detection of p38 MAPK Phosphorylation and Intracellular Cytokine Expression in Peripheral Blood Subpopulations from Patients with Autoimmune Rheumatic Diseases” Journal of Immunology Research Vol. 2014 (2014), p13 (available at http://www.hindawi.com/journals/jir/2014/671431/).

Fluorescence Spectroscopy

Fluorescence spectroscopy is an electromagnetic spectroscopy which analyzes fluorescence from a sample. It uses a beam of light (most commonly used is ultraviolet light), exciting the electrons in the molecules of compounds and causes emission of light. An example of the use of fluorescence spectroscopy in the study of the MAPK signaling pathway is provided in D. B. Slaughter; J. W. Schwartz; R. Li. “Mapping dynamic protein interactions in MAP kinase signaling using live-cell fluorescence fluctuation spectroscopy and imaging,” Proc. Natl. Acad. Sci. Dec. 18, 2007; 104(51), pp. 20320-25. (available at http://www.ncbi.nlm.nih.gov/pmc/articles/PMC2154429/#_ffn_sectitle).

Surface Plasmon Resonance (SPR)

Excited by incident light, surface plasmon resonance (SPR) is the accumulation of oscillation of electrons. It constitutes a powerful method and permits a real-time, label free detection of biomolecular interactions. SPR generates plasmons by hitting polarized light on an electrically conducting surface at the interface between two media causing reflected light's intensity diminution at a particular angle (resonance angle). Plasmons are proportional to the mass on a sensor surface. An example of the use of SPR in the study of the Hedgehog signaling pathway is provided in F. Zhang; J. S. McLellan; A. M. Ayala; D. J. Leahy; R. J. Linhardt, “Kinetic and structural studies on interactions between heparin or heparan sulfate and proteins of the hedgehog signaling pathway,” Biochemistry, 2007 Apr. 3; 46(13), pp. 3933-41 (available at http://www.ncbi.nlm.nih.gov/pubmed/17348690).

Calorimetry

Calorimetry is the amount of heat measurement associated with chemical reaction changes such as physical changes and phase transitions which are accompanied by heat transfer. Heat can be generated (exothermic), consumed (endothermic), or dissipated. An example of the use of calorimetry in the study of the RTK signaling pathway is provided in A. M. Spurches; H. J. Argiros; K. H. Lee; L. L. Haas; S. C. Pero; D. N. Krag; P. P. Roller; D. E. Wilcox; B. a. Lyions, “Calorimetric investigation of phosphorylated and non-phosphorylated peptide ligand binding to the human Grb7-SH2 domain,” J Mol Recognit. 2007 July-August; 20(4), pp. 245-52. (available at http://www.ncbi.nlm.nih.gov/pubmed/17705331)

Modeling of Signaling Networks

The scale, nonlinearities, and interconnected complexity of biochemical signaling networks have been initially addressed with attempts to modularize. A newly popular approach is the partition of complex signaling networks into small sections that are characterized as behaving like combinational and state-retaining logic circuits, i.e., so-called signaling “motifs.” However, the scale, interconnected complexity, and adaptively of biochemical signaling networks exceeds human comprehension and defies attempts to modularize. In many cases, conceptual modeling of biological signaling network has proven ineffective and at time deceptive as it is mentally impossible to juggle large pathways involving many components and because the mathematical behavior is too hard to intuitively characterize. In many ways the predictive modeling of signaling networks has exceeded what can be done without computer support.

Further, the explosion of genomic and proteomic laboratory analysis, bioinformatics, research publications, and “big data” analysis has created vast tomes of unverified signaling network models. Many of these are the result of automated statistical analysis of the results of automatic inferences drawn from automatic word searches on research publications text, be they speculative, unconfirmed, or robust verified.

Accurate Measurement of Rate Constants

To the extent that there is some valid degree of topological accuracy, reaction rates and many other parameters required for accurate analytical modeling are often unavailable. Further, the measurements of reaction rates and other parameters required for accurate analytical modeling is often made under artificial and inaccurate circumstances, for example not including the profound effects of molecular crowding, localized and confined reaction environments, or even being able to accurately control for other potentially interfering processes.

Summarizing

Summarizing some of the points thus far together with some additional remarks:
The long celebrated and indoctrinated methods of drug design and testing are failing to produce and producing exploding costs;
For many reasons the best candidates for the next step is the leveraging of analytical, quantitative, confirmative, and predictive understanding of biochemical signaling process, pathways, and networks;
For this and yet other reasons, an extensive analytical, quantitative, confirmative, and predictive understanding of biochemical signaling process, pathways, and networks is becoming increasingly indispensable for drug design, therapies, treatment of disease, and the control of destructive side effects;
The scale, nonlinearities, interconnected complexity, and adaptively of biochemical signaling networks exceeds human comprehension and defies attempts to modularize;
The need for accurate large-scale computer models of biochemical networks is urgent as there is vast need for predictive modeling of signaling networks, yet the predictive modeling has exceeded what can be done without computer support;
However, the powerful tools and methods employed to date for characterizing signaling processes, signaling network topologies, and signaling process modeling parameters are still primitive and subject to immense error.

As to some of the powerful tools and methods employed to date for characterizing biochemical signaling processes, signaling network topologies, and signaling process modeling parameters, FIG. 1 depicts an example spectrum of tools used in the study of biochemical signaling networks. Not explicitly depicted is the combined use of genomic and proteomic laboratory analysis, bioinformatics, research publications, and “big data” analysis in the creation of huge unverified signaling network models from automated statistical analysis of the results of automatic inferences drawn from automatic word searches on research publications text, be they speculative, unconfirmed, or robust verified; if these are to be included for some sense of completeness (as it is arguably a huge area of recent activity) these could fit in the meeting ground between the depicted group categories of “physical” and “mathematical” methodologies. The intended point of FIG. 1, however, is that there is a spectrum of methodologies ranging from attempts to directly and accurately observe biochemical signaling processes, signaling network topologies, and signaling process modeling parameters on one extreme to attempts to directly and accurately model with abstract mathematical models that hold the promise of providing predictive analytical and design tools.

In mechanical, electrical, optical, chemical, material science, and other forms of engineering there has been great success in creating abstract mathematical models (“CAD tools”) that accurate provide predictive analytical and design tools. These tools incorporate vast amounts of confirmed physical science, accurate physical measurements, and confirmed analytical models combined into a comprehensive framework, and virtually no contemporary suspension bridge, transportation vehicle, integrated circuit, consumer product, or chemical plant is designed without such tools. However, such tools have not yet become possible because of the shortcomings and shortages of needed confirmed physical science, needed accurate physical measurements, needed confirmed analytical models, and needed comprehensive framework.

Each of the five examples cited in the example spectrum of tools used in the study of biochemical signaling networks shown in FIG. 1 are subject to a number of limitations as described earlier. For example, the physical measurements of reaction rates and other parameters required for accurate analytical modeling is often made under artificial and inaccurate circumstances, for example not including the profound effects of molecular crowding, localized and confined reaction environments, or even being able to accurately control for other potentially interfering processes. On the other side of the spectrum, computer simulations and analytical models suffer from, among other things, the lack of accurate physical measurements, confirmed accurate analytical models, adequate level of scale, accurate signaling network topologies, and overall comprehensive framework.

### SUMMARY

The present application is directed to creating new tools and methods of research in the areas of biochemical signaling, rate constant determination, protein interaction, modeling, disease processes, drug discovery, cell biology, and other applications.

For purposes of summarizing, certain aspects, advantages, and novel features are described herein. Not all such advantages may be achieved in accordance with any one particular embodiment. Thus, the disclosed subject matter may be embodied or carried out in a manner that achieves or optimizes one advantage or group of advantages without achieving all advantages as may be taught or suggested herein.

The approach adapts the concept of a “breadboard” such as the electronic breadboards used in electronic circuitry prototyping and optical breadboards used in optical system R&D. The term breadboard comes from early experimental engineering practice where experimental electronics circuits were literally built on refined pieces of wood used for slicing bread, or pieces of wood suggestive of such, spreading the locations of electronic components across the surface of the wood and interconnecting the leads of the electronic components by connecting them to screws or nails put into the wood where needed. Often such a “breadboarded” electronic circuit was only a portion of a larger system. Today more formalized and structured breadboarding environments are available for electronics and optics, and typically these are richly interconnected to a number of external measurement devices, signal sources, and controlling devices. It is an analogy with many of these aspects that the term “breadboard” is used here. Other analogies could be made by viewing the present invention as a controlled (and/or instrumented) fluidics-based assay plate or a controlled (and/or instrumented) microfluidics version of a test tube.

The present invention includes approaches for the selective piecewise construction of replicas of portions of naturally-occurring biochemical processes and pathways for signaling, metabolism, and gene regulation.

These replicas can for example be implemented in microscale and nanoscale fluidic environments, can internally comprise one or more microscale and nanoscale fluidic environments, can be computer-controlled, and can comprise extensive monitoring via internal sensors, external sensors, and other types of instrumentation.

The replicas can for example additionally be arranged to include a variety of constituent species such as enzymes, other proteins, lipids, ions, peptides, and other materials, and the introduction of such constituent species can be provided under controlled conditions under controlled timing.

The replicas can for example additionally be arranged to include the controlled presence and controlled introduction of varying degrees of competitive species, drugs, and environmental influences (hormone, temperature, chemical, etc.).

Further, and in many cases importantly, the replicas can be arranged to include controlled degrees of substitute or representative molecular crowding.

The embodiments of the present application are directed to address problems in the study, analysis, and modeling of biological signaling processes and networks in living biological cells. Because of this and the “breadboard” abstraction adopted from electronics prototyping and optical R&D, for convenience embodiments of the present application will be referred to as a “biological signaling breadboard.” Embodiments of the present application can be readily used or adapted for the study, analysis, and modeling of biochemical processes and pathways for metabolism and gene regulation.

Embodiments of the biological signaling breadboard or features therein can comprise one or more microscale or nanoscale chemical reaction environments, each for example chemical reaction environment arranged to:
accept reactants, reagents, and other material,
comprise at least one reaction environment,
include or support sensors or internal instrumentation for monitoring one or more of:
   the presence or concentration of chemical/biochemical species,
   the presence and progress of chemical/biochemical processes,
include or support aspects of external instrumentation for monitoring one or more of:
   the presence or concentration of chemical/biochemical species,
   the presence and progress of chemical/biochemical processes,
provide controlled introduction of one or more chemical/biochemical materials,
provide controlled stimulus to initiate or maintain one or more chemical/biochemical processes.

Embodiments of the biochemical signaling breadboard can further be configured to interface with a computing system performing one or more of the following functions:
Receive measurement information from sensors and/or instrumentation associated with each of the one or more microscale or nanoscale chemical reaction environments;
Transmit control information used to control fluidics systems.
Transmit control information used to control the introduction of one or more chemical/biochemical materials into each of the one or more microscale or nanoscale chemical reaction environments;
Transmit control information used to control the stimulus of one or more chemical/biochemical processes into each of the one or more microscale or nanoscale chemical reaction environments;
Execute control algorithms for creating and timing the transmitting of the aforementioned control information;
Execute feedback control algorithms for creating and timing the transmitting of the aforementioned control information responsive to received measurement information;
Execute storage algorithms for at least storing the aforementioned measurement information to create stored measurement information;
Execute retrieval algorithms for at least retrieving the aforementioned stored measurement information;
Execute control algorithms for creating and timing the transmitting of the aforementioned control information responsive to retrieved stored measurement information;
Execute analysis algorithms for at least analyzing the aforementioned measurement information;
Execute storage algorithms for at least storing the aforementioned analysis information to create stored analysis information;
Execute retrieval algorithms for at least retrieving the aforementioned stored analysis information;
Execute control algorithms for creating and timing the transmitting of the aforementioned control information responsive to retrieved stored analysis information;
Support the use of scripts and script-driven control algorithms;
Provide user interface functions.
In some embodiments the computing system is also executing a mathematical model, for example as part of the aforementioned algorithms or in communication with the aforementioned algorithms

In embodiments where there are more than one microscale or nanoscale chemical reaction environments, where advantageous, two or more of these microscale or nanoscale chemical reaction environments can be configured to comprise linking algorithms or other arrangements wherein received measurement information associated with one of the microscale or nanoscale chemical reaction environments is used for creating and timing the transmitting of control information directed to at least one other of the microscale or nanoscale chemical reaction environments.

In embodiments comprising at least one such linking algorithm, the combination of controlled and (sensor or instrument) monitored microscale or nanoscale chemical reaction environments together with the linking algorithms can be used to emulate a signaling cascade or portion of a signaling network.

In embodiments comprising a plurality of such linking algorithm, the combination of controlled and (sensor or instrument) monitored microscale or nanoscale chemical reaction environments together with the linking algorithms can be used to emulate a larger signaling cascade, portion of a signaling network, or entire signaling network.

In embodiments comprising a plurality of such linking algorithms, and wherein the linking introduces a feedback loop involving two or more of the microscale or nanoscale chemical reaction environments, the combination of controlled and (sensor or instrument) monitored microscale or nanoscale chemical reaction environments together with the linking algorithms can be used to emulate signaling feedback, a signaling cascade with feedback, a portion of a signaling network comprising feedback, or entire signaling network comprising feedback.

In embodiments comprising a plurality of such linking algorithms, and wherein the linking introduces a feedforward path involving two or more of the microscale or nanoscale chemical reaction environments, the combination of controlled and (sensor or instrument) monitored microscale or nanoscale chemical reaction environments together with the linking algorithms can be used to emulate signaling feedforward, a signaling cascade with feedforward, a portion of a signaling network comprising feedforward, or entire signaling network comprising feedforward paths.

Embodiments of the biochemical signaling breadboard can further be configured to include at least one fluidic interconnection between at least two of the microscale or nanoscale chemical reaction environments. In some embodiments the fluidic interconnection is simply gated on and off under computer control. In other embodiments, the fluidic interconnection is realized as one possible configuration of a computer-controlled reconfigurable fluidic interconnection network. In some embodiments the computer-controlled reconfigurable fluidic interconnection network can be implemented as or comprise aspects of a controllable multichannel microfluidic chemical bus such as that taught in pending U.S. Pat. Nos. 8,032,258 and 8,606,414 and pending U.S. patent application Ser. No. 13/251,288.

In embodiments comprising at least one such fluidic interconnection, the combination of controlled and (sensor or instrument) monitored microscale or nanoscale chemical reaction environments together with the linking algorithms can be used to emulate a signaling cascade or portion of a signaling network.

In embodiments comprising a plurality of such fluidic interconnections, the combination of controlled and (sensor or instrument) monitored microscale or nanoscale chemical reaction environments together with the linking algorithms can be used to emulate a larger signaling cascade, portion of a signaling network, or entire signaling network.

In embodiments comprising a plurality of such fluidic interconnections, and wherein the linking introduces a feedback loop involving two or more of the microscale or nanoscale chemical reaction environments, the combination of controlled and (sensor or instrument) monitored microscale or nanoscale chemical reaction environments together with the linking algorithms can be used to emulate signaling feedback, a signaling cascade with feedback, a portion of a signaling network comprising feedback, or entire signaling network comprising feedback.

In embodiments comprising a plurality of such fluidic interconnections, and wherein the linking introduces a feedforward path involving two or more of the microscale or nanoscale chemical reaction environments, the combination of controlled and (sensor or instrument) monitored microscale or nanoscale chemical reaction environments together with the linking algorithms can be used to emulate signaling feedforward, a signaling cascade with feedforward, a portion of a signaling network comprising feedforward, or entire signaling network comprising feedforward paths.

### BRIEF DESCRIPTION OF THE DRAWINGS

The above and other aspects, features and/or advantages of the present application may become more apparent upon consideration of the following description of embodiments taken in conjunction with the accompanying drawing figures, wherein:

FIG. 1 illustrates a spectrum of tools used in the study of biochemical signaling networks.

FIG. 2 illustrates a relative role of an embodiment of the present application in the spectrum depicted in FIG. 1.

FIG. 3 illustrates a representation of recent understanding of the EGF (Epidermal Growth Factor) induced MAP (Mitogen-Activated Protein) Kinase Signal Transduction Pathway.

FIG. 4 illustrates an example representative pathway segment such as that found in biochemical signaling pathways.

FIG. 5a illustrates an example partition of the example representative pathway segment depicted in FIG. 4.

FIG. 5b illustrates another example partition of the example representative pathway segment depicted in FIG. 4.

FIG. 5c illustrates the entire example representative pathway segment depicted in FIG. 4.

FIG. 6 depicts some examples of how the various steps in biochemical pathways such as those depicted in FIGS. 3, 4, and 5a-5c can be classified in a manner suitable for the present invention or extensions of the present invention.

FIG. 7a depicts the implementation of a stoichiometric replica reaction emulating a biochemical pathway step wherein the emulating reaction only involves constituent materials.

FIG. 7b depicts the implementation of a stoichiometric replica reaction that additionally involves one or more inhibitors, catalysts, or other reaction-affecting agents.

FIG. 8 shows an example of a “blockwise” replica reaction that consumes reaction product B.

FIGS. 9a and 9b depict an example splitting of the “blockwise” replica reaction of FIG. 8 wherein a consuming measurement of reaction product A is made for the “stepwise” replica reaction depicted in FIG. 9a, and controlled introduction of that reaction product A is (then or later) provided by computer control as a constituent material into the “stepwise” replica reaction depicted in FIG. 9b.

FIG. 10 depicts an example arrangement wherein the “stepwise” replica reactions depicted in FIGS. 9a and 9b are joined together by a replication process.

FIG. 11 illustrates an example comparison of the present invention to other techniques used in studying the biochemistry and phenomenology of biochemical pathways.

FIG. 12 depicts a simple example of a signaling pathway step emulation employing external computer controlled precision micro-flow stepper-motor pumps such as those commonly used in precision liquid chromatography to provide controlled application of constituents.

FIG. 13 depicts a variation on the arrangement suggested by FIG. 12 wherein on-chip microfluidic pumps are used to provide controlled application of constituents.

FIG. 14 illustrates an example embodiment comprising a plurality of computer-controlled and computer-monitored microscale or nanoscale chemical reaction environments and a computer-controlled arrangement for executing algorithms and interfacing with the plurality of computer-controlled and computer-monitored microscale or nanoscale chemical reaction environments.

FIG. 15 illustrates an example approach to emulating molecular crowding through the controlled introduction of molecular crowding constituents into a monitored microscale or nanoscale chemical reaction environment.

FIG. 16 illustrates an example variation on the example embodiment depicted in FIG. 14 that incorporates the approach to emulating molecular crowding depicted in FIG. 15.

FIG. 17 illustrates an example variation on the approach to emulating molecular crowding depicted in FIG. 15.

FIG. 18 illustrates an example variation on the example embodiment depicted in FIG. 16 that incorporates the approach to the dispensing of drug constituents depicted in FIG. 17.

FIG. 19 through FIG. 34 and the following tables provide example sequential step-by-step breakdown of an example contemporary understanding of the Wnt pathway.

FIG. 35 through FIG. 40 and the following tables provide example sequential step-by-step breakdown of an example contemporary understanding of the Hedgehog pathway.

FIG. 41 through FIG. 56 and the following table provide example sequential step-by-step breakdown of an example contemporary understanding of the RTK pathway.

FIG. 57 through FIG. 64 and the following table provide example sequential step-by-step breakdown of an example contemporary understanding of the JAK-STAT pathway.

FIG. 65 through FIG. 72 and the following table provide example sequential step-by-step breakdown of an example contemporary understanding of the Notch pathway.

### DETAILED DESCRIPTION

In the following description, reference is made to the accompanying drawing figures which form a part hereof, and which show by way of illustration specific embodiments of the present application. It is to be understood by those of ordinary skill in this technological field that other embodiments may be utilized, and structural, electrical, as well as procedural changes may be made without departing from the scope of the present application.

In the following description, numerous specific details are set forth to provide a thorough description of various embodiments. Certain embodiments may be practiced without these specific details or with some variations in detail. In some instances, certain features are described in less detail so as not to obscure other aspects. The level of detail associated with each of the elements or features should not be construed to qualify the novelty or importance of one feature over the others.

The present disclosure describes approaches to additional tools for the study of biological cell signaling employing methods, microscale sensors, microscale instrumentation, microfluidic, and computerized instrumentation. The approach adapts the concept of a “breadboard” such as the electronic breadboards used in electronic circuitry prototyping and optical breadboards used in optical system R&D. Embodiments of the present application will be referred to as “biological signaling breadboards” for convenience. Results from and the environments comprised by embodiments of the present application can be used by and combined with computer simulation and computer analysis tools relating to the study, analysis, and modeling of biological cell signaling, for example the modeling environment described in U.S. Pat. No. 8,660,823 by the present inventor. For example, results from and the environments comprised by embodiments of the present application can be used by and combined with computer simulation and computer analysis tools in manners such as those described in pending U.S. patent application Ser. No. 13/157,304.

Among other things, the present disclosure describes approaches for selective piecewise construction of replicas of portions of naturally-occurring biochemical processes and pathways for signaling, metabolism, and gene regulation. These replicas can for example be implemented in microscale and nanoscale fluidic environments, can internally comprise one or more microscale and nanoscale fluidic environments, can be computer-controlled, and can comprise extensive monitoring via internal sensors, external sensors, and other types of instrumentation. The replicas can for example additionally be arranged to include a variety of constituent species such as enzymes, other proteins, lipids, ions, peptides, and other materials, and the introduction of such constituent species can be provided under controlled conditions under controlled timing. The replicas can for example additionally be arranged to include the controlled presence and controlled introduction of varying degrees of competitive species, drugs, and environmental influences (hormone, temperature, chemical, etc.). Further, the replicas can be arranged to include controlled degrees of substitute or representative molecular crowding.

In embodiments where there are more than one microscale or nanoscale chemical reaction environments, where advantageous, two or more of these microscale or nanoscale chemical reaction environments can be configured to comprise linking algorithms or other arrangements wherein received measurement information associated with one of the microscale or nanoscale chemical reaction environments is used for creating and timing the transmitting of control information directed to at least one other of the microscale or nanoscale chemical reaction environments.

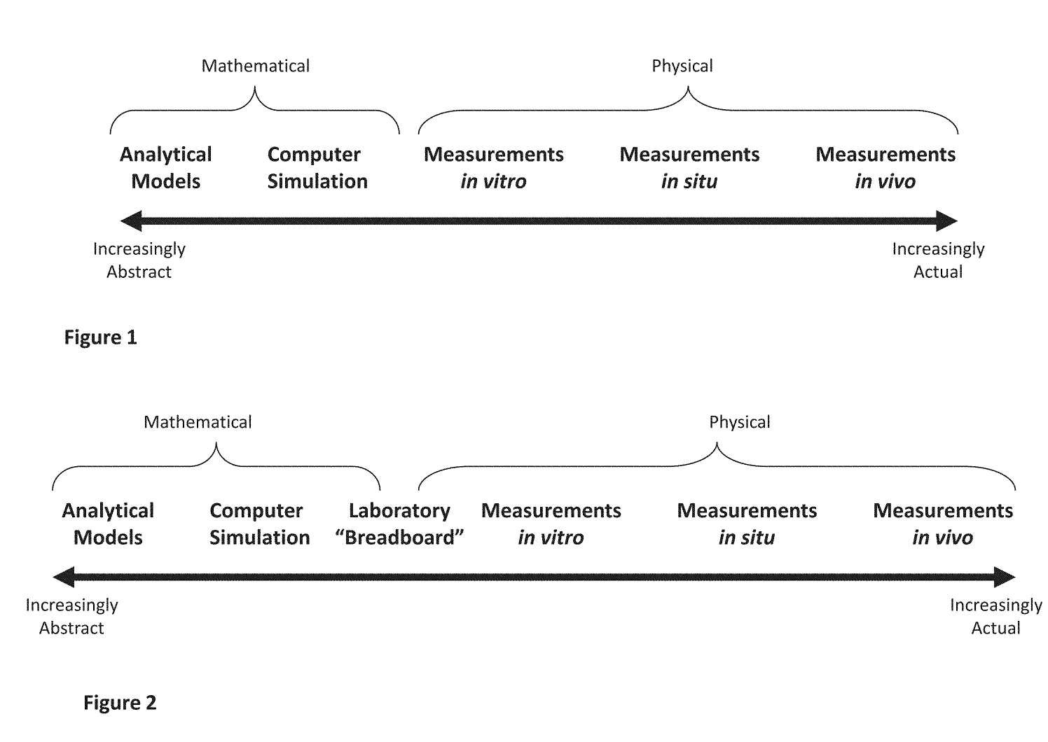

The embodiments of the present application are directed to the study, analysis, and modeling of biological cell signaling, and also could be used for the study, analysis, and modeling of biochemical processes and pathways for metabolism and gene regulation. In that it can combine physical measurements with computer control driving by mathematical models, FIG. 2 depicts an example relative role of an embodiment of the present application straddling “physical” and “mathematical” methodologies in the spectrum depicted in FIG. 1.

Additionally, aspects of the present application can also be readily used or adapted for laboratory or industrial activities pertaining to confined-environment chemistry, intercalation chemistry, chemical reactions in constrained systems, molecular encapsulation, and aspects of host-guest chemistry and the synthesis and study of meta-materials.

Naturally-Occurring Biochemical Signaling Pathways

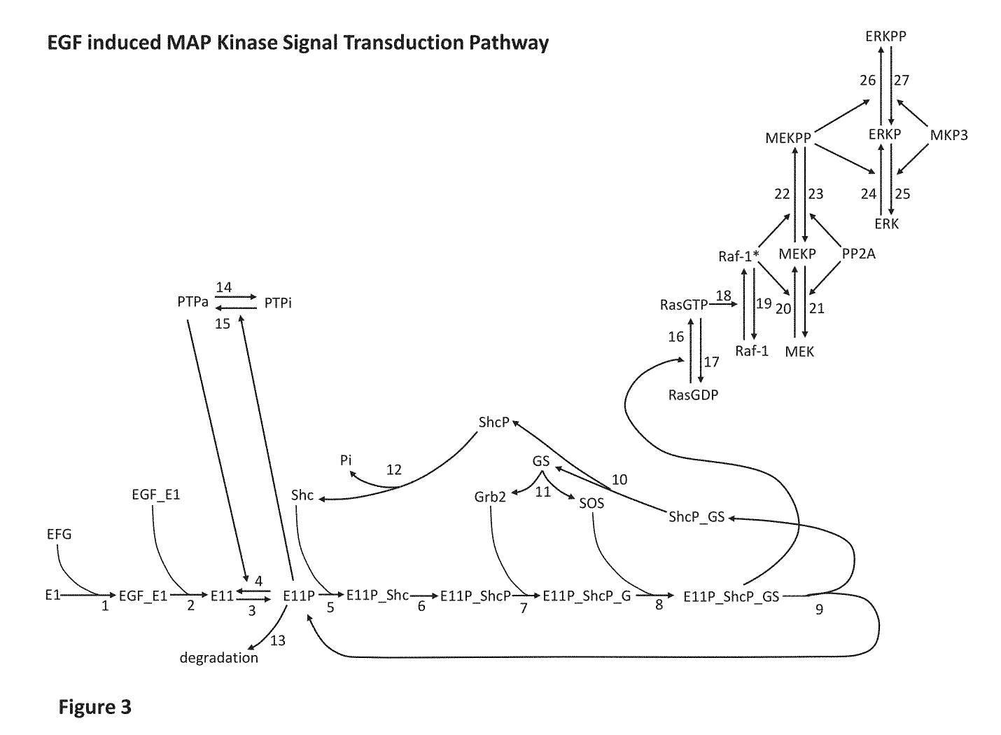

As described above, network topology graphs (the term “graph” here being the mathematical term meaning a collection of nodes and a collection of links or “edges” interconnecting these nodes) for naturally-occurring biochemical signaling pathways can be quite large and are always subject to change subject to new findings and their acceptance. As a representative example, FIG. 3 depicts a representation of recent understanding of the EGF (Epidermal Growth Factor) induced MAP (Mitogen-Activated Protein) Kinase Signal Transduction Pathway, one of the most important pathways in mammalian cells for regulating cell growth, survival, proliferation, and differentiation. Accordingly, this is one of the most experimentally and computationally investigated cellular signaling pathways, with numerous dynamic analysis and computational models available in the literature. (A popular review article on this pathway providing extensive citations is that of Oda, Matsuoka, Funahashi, Kiano “A Comprehensive Pathway Map of Epidermal Growth Factor Receptor Signaling,” Molecular Systems Biology, 1:2005.0010, May 25, 2005.)

This and other pathways could be separated or segmented into interconnected smaller portions of the overall pathway with, for example, pair-wise interfaces among smaller portions of the overall pathway, these pair-wise interfaces implementing or supporting the interconnection. Several additional example pathways implicated in many human diseases, and detailed partition of these into smaller portions or segments, are provided in later in the discussion.

Leveraging this general approach provides a basis for creating a “biological signaling breadboard” as will be described. A larger pathway can be separated or segmented into interconnected smaller portions, at least one of which can be to some degree of controlled approximation be accurately emulated with some form of replica microscale and/or nanoscale fluidic implementation whose constituent species can be closely controlled and at least one aspect of whose biochemical behavior can be closely measured by some means with adequate accuracy. The control and measurement information can be interfaced with a computer that executes algorithms comprising for example one or more of a control process, control event-script, experiment, data recording, and mathematical model.

Example Partitions of Natural Biochemical Signaling Pathways

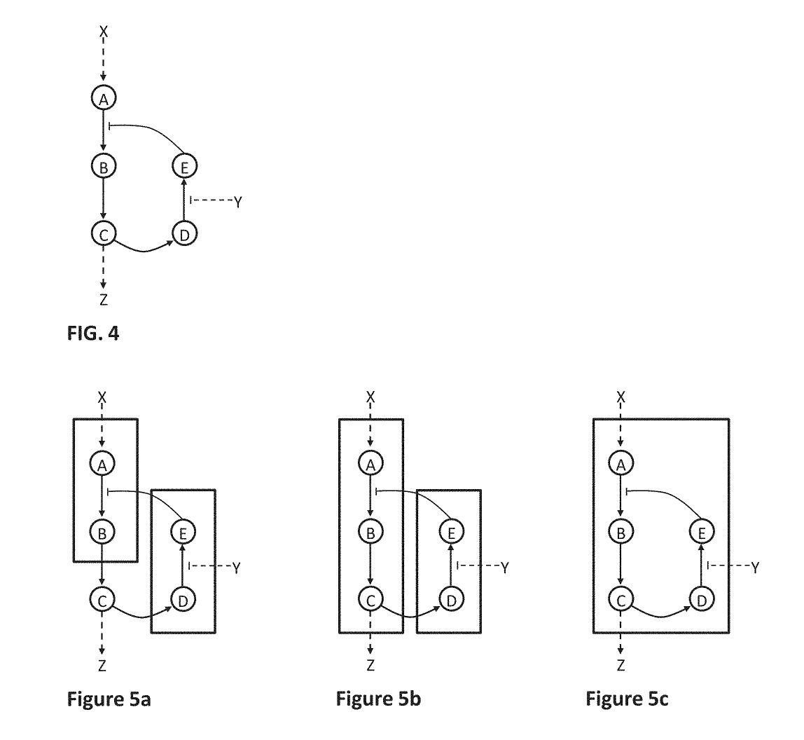

As an abstract example, FIG. 4 depicts a representative pathway segment such as that found in biochemical signaling pathways. The constituent materials A, B, C, D, and E can be, for example, proteins, ions, ligands, complexes, etc. In this example, an incoming stimulus from external source X introduces a constituent material A, which subsequently reacts with or activates a constituent material B, which in turn produces or activates C, the results of which is provided to both external respondent Z as well as internal respondent constituent material D, the latter which in turn reacts with or activates E subject to inhibition by material or influence provided by external source Y. The reaction with or activation of E in turn inhibits the reaction with or activation of A.

FIG. 5a depicts an example partition of the example representative pathway segment depicted in FIG. 4, wherein a first partition comprises constituent materials A and B, the pathway from A to B, and an inhibitor input to that pathway from A to B, and wherein a second partition comprises constituent material D and E, the pathway from D to E, and an inhibitor input to the pathway from D to E. The partition excludes constituent material C.

FIG. 5b depicts another example partition of the example representative pathway segment depicted in FIG. 4, wherein a first partition comprises constituent materials A, B, C, the pathway between A and B, an inhibitor input to the pathway from A to B, and the pathway from B to C.

FIG. 5c depicts the entire example representative pathway segment depicted in FIG. 4, so the partition is simply the separation for external sources X and Y and external respondent Z.

In an embodiment of the present invention or extensions of the present invention, the entire example representative pathway segment depicted in FIG. 4 can be “step-wise” implemented with computer-controlled and computer-monitored microscale or nanoscale chemical reaction environments linked to at least one algorithm executing on a computer, and physically provided concentrations of constituent materials that would be provided by X and Y are instead controlled by at least one algorithm executing on the computer or a related computer. For example, for either of the cases depicted in FIG. 5a or FIG. 5b, a first computer-controlled and computer-monitored microscale or nanoscale chemical reaction environment can be used to implement the left-side partition depicted in the figure and a second computer-controlled and computer-monitored microscale or nanoscale chemical reaction environment can be used to implement the right-side partition depicted in the figure. The first and second computer-controlled and computer-monitored microscale or nanoscale chemical reaction environments can be separately studied, or linked by using measurements made of the measurable product or outcome of the first computer-controlled and computer-monitored microscale or nanoscale chemical reaction environment to provide controlled introduction and concentration of that measurable product or outcome to the second computer-controlled and computer-monitored microscale or nanoscale chemical reaction environment. The stimulus to external respondent Z can, for example be represented by a measurement of the concentration or other properties of constituent material C.

In another embodiment of the present invention or extensions of the present invention, the entire example representative pathway segment depicted in FIG. 4 can be can be “step-wise” implemented with computer-controlled and computer-monitored microscale or nanoscale chemical reaction environments linked to at least one algorithm executing on a computer, and physically provided concentrations of constituent materials that would be provided by X and Y are instead controlled by at least one algorithm executing on the computer or a related computer. For example, for either of the cases depicted in FIG. 5a or FIG. 5b, a first computer-controlled and computer-monitored microscale or nanoscale chemical reaction environment can be used to implement the left-side partition depicted in the figure and a second computer-controlled and computer-monitored microscale or nanoscale chemical reaction environment can be used to implement the right-side partition depicted in the figure, and the first and second computer-controlled and computer-monitored microscale or nanoscale chemical reaction environments can be linked by at least one algorithm executing on a controlling computer or a related computer. The stimulus to external respondent Z can, for example be represented by a measurement of the concentration or other properties of constituent material C. The combination of two or more emulation linked by an algorithm in such a manner, or related manner, while be referred to as an “emulation-simulation hybrid.”

In yet another embodiment of the present invention or extensions of the present invention, the entire example representative pathway segment depicted in FIG. 4 can be can be “block-wise” implemented with a single computer-controlled and computer-monitored microscale or nanoscale chemical reaction environment linked to at least one algorithm executing on a computer, and physically provided concentrations of constituent materials that would be provided by X and Y are instead controlled by at least one algorithm executing on the computer or a related computer. For example, the case depicted in FIG. 5c can be implemented in a single “one-pot” computer-controlled and computer-monitored microscale or nanoscale chemical reaction environment. The stimulus to external respondent Z can, for example be represented by a measurement of the concentration or other properties of constituent material C.

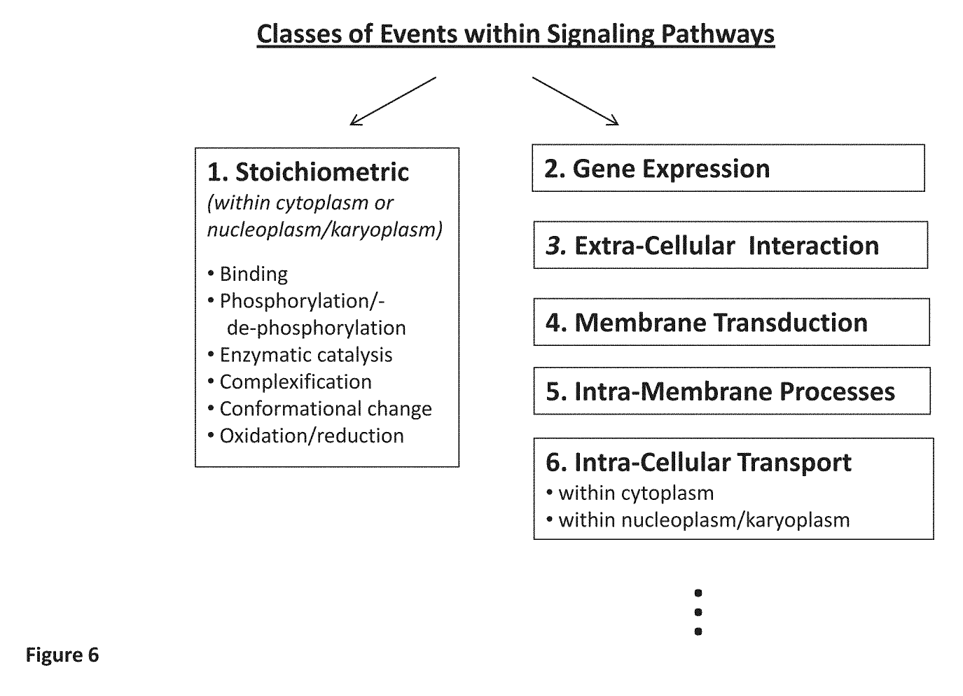

Various steps in biochemical pathways such as those depicted in FIGS. 3, 4, and 5a-5c can comprise many aspects. FIG. 6 depicts some examples of how the various reaction steps and other steps making up biochemical pathways such as those depicted in FIGS. 3, 4, and 5a-5c can be classified in a manner suitable for the present invention or extensions of the present invention. The class on the left side of the figure captures chemical reactions and other processes that can readily occur in a liquid media either without any additional physical structure or with suitable microenvironments as can be implemented with, for example, structured polymers, graphene and graphite structures, pillared clays and other types of structured clays, structured synthetic zeolites, inclusion compounds and cavity-containing supra-molecular compounds (for example cyclodextrins, calixarenes, and other organic host lattices), clathrates, liposomes, and various types of self-assembled supra-molecular structures. This class, for example, can apply to many steps in biochemical pathways occurring in the cytoplasm or nucleoplasm (a.k.a “karyoplasm”), and can be readily implemented in a simple unstructured reaction chamber wherein constituent reactants are simply mixed together in a media. For the purposes of discussion, this class will be referred to as “stochiometric” (with reference to stoichiometry as calculation of quantities of reactants and products in chemical reactions). Further, as will be discussed, the reaction chambers and media within them can be configured in some embodiments to accept and include controlled introductions of molecular crowding materials, drugs, gases, and other substances.

Accordingly, implementation of reaction chambers provided with continent materials in (fluid and/or gas) media and supporting reactions of these constituents thus provide a means of creating “replica reaction” steps that can emulate steps in natural biochemical pathways. Further, with increasing levels of precision made possible by various aspects of the invention and its implementation, steps in biochemical pathways can be increasingly accurately emulated by these reaction chambers.

Measurement of the concentration or other properties of product or other target constituent material(s) created or affected in the reaction chamber can be used to monitor the “replica reaction” and serve as output of emulations of one or more steps in biochemical pathways emulated by these reaction chambers. Although in principle biochemical pathway reaction steps can be freely defined and reorganized, when the invention is configured so that a reaction chamber implements a single reaction step (which can involve one or more constituents and can, as appropriate, include inhibitors and catalysis as well as reactants), the invention will be said to be implementing a “stepwise” emulation, and when the invention is configured so that a reaction chamber implements a plurality of reaction steps in the same reaction chamber, the invention will be said to be implementing a “stepwise” emulation.

By implementing the reaction chambers and associated material transport with microfluidic and microfluidic-like technology, or with nanofluidic and nanofluidic-like technology, the quantities of constituent material (which can be rare, expensive, or require custom derivation or synthesis) can be kept small and a wide variety of microfluidic and nanofluidic sensing methods can be used. For example, sensing of reaction products, binding, molecular states, etc. in the recation chambers can be performed using optical, spectroscopic, and/or electrical techniques such as fluorescent markers, spin traps, chromaphores, antibodies markers, antibody-based bioFETs, enzyme-based bioFETs, chemFETs, other electrochemical sensors, etc., as well as a wide range of other current, evolving, emergent, and future techniques.

Further, with the proper use of for example, structured polymers, graphene and graphite structures, pillared clays and other types of structured clays, structured synthetic zeolites, inclusion compounds and cavity-containing supra-molecular compounds (for example cyclodextrins, calixarenes, and other organic host lattices), clathrates, liposomes, and various types of self-assembled supra-molecular structures, the simple reaction chambers can be structured to emulate various types of micro-reaction environments, for example as occur in cell mitochondria.

Regarding the example additional classes of processes depicted in the right side of FIG. 6 (namely gene expression, extra-cellular interaction, membrane transduction, intra-membrane processes, inter-cellular transport, etc.), similar methods or entirely different methods can be used to create emulations of these processes. The emulation of these and other classes of processes will be subject of companion patent applications.

The present invention provides for two replica stoichiometric reactions that are separated by a shared non-stoichiometric process to simulate the effect of the non-stoichiometric process, for example employing an emulation-simulation hybrid as defined above.

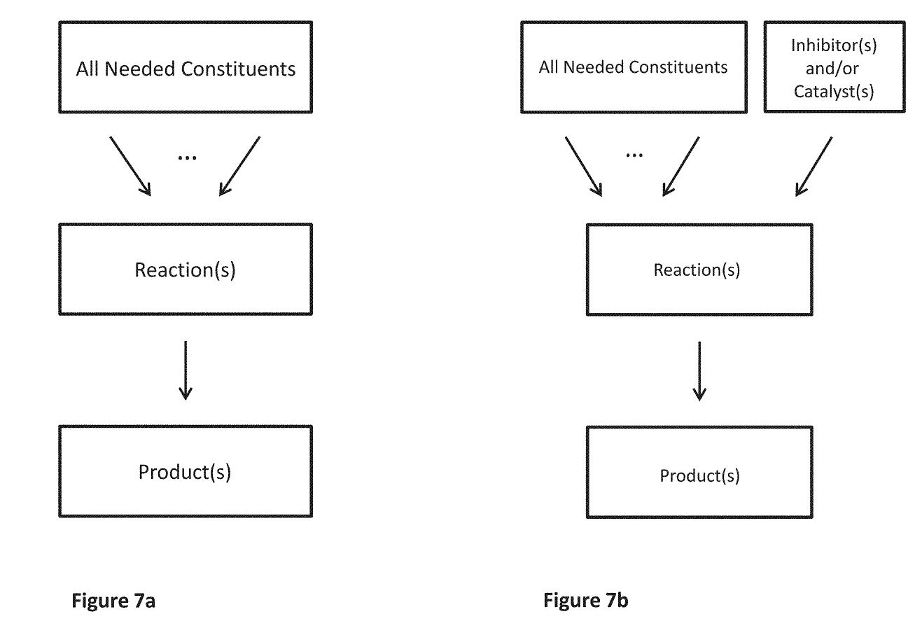

Turning attention now largely to the implementation of stoichiometric replica reactions, FIG. 7a depicts the implementation of a stoichiometric replica reaction emulating a biochemical pathway step wherein the emulating reaction only involves constituent materials. Alternatively, the implementation of a stoichiometric replica reaction can additionally involve one or more inhibitors, catalysts, or other reaction-affecting agents as suggested in FIG. 7b.

Regarding measurement, although some measurement techniques do not affect reaction products, constituents, or other reaction agents, many useful measurement methods will consume reaction products, constituents, or other reaction agents—for example sensing methods involving antibodies that bind to reaction products, constituents, or other reaction agents in order to detect them.

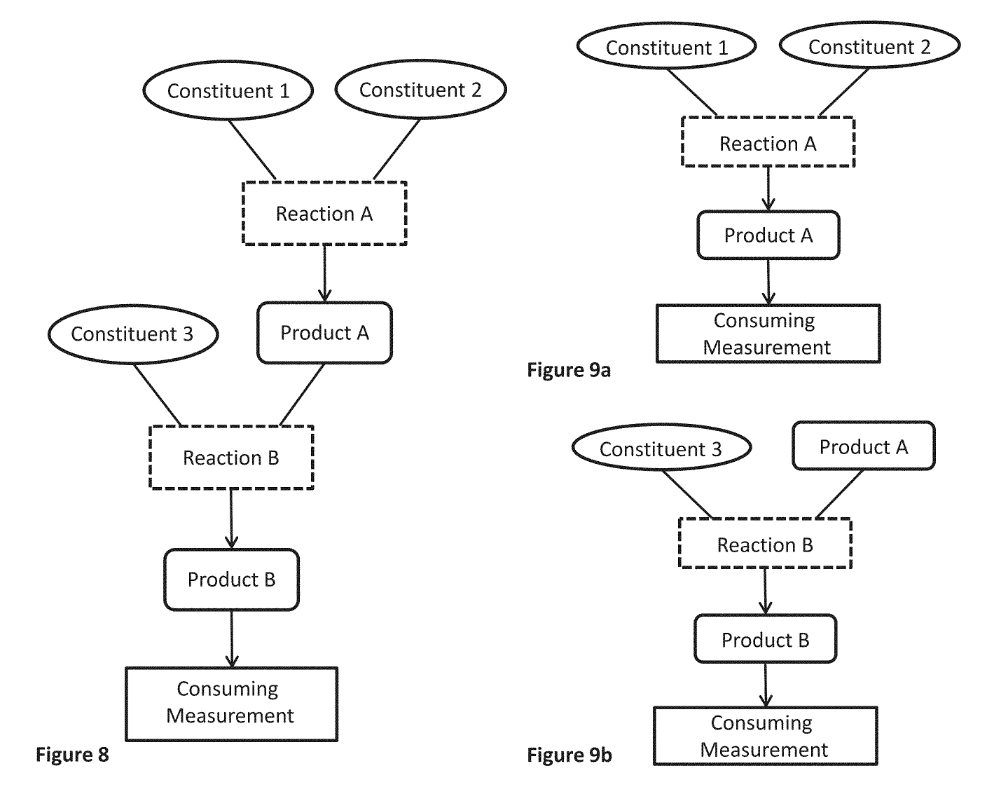

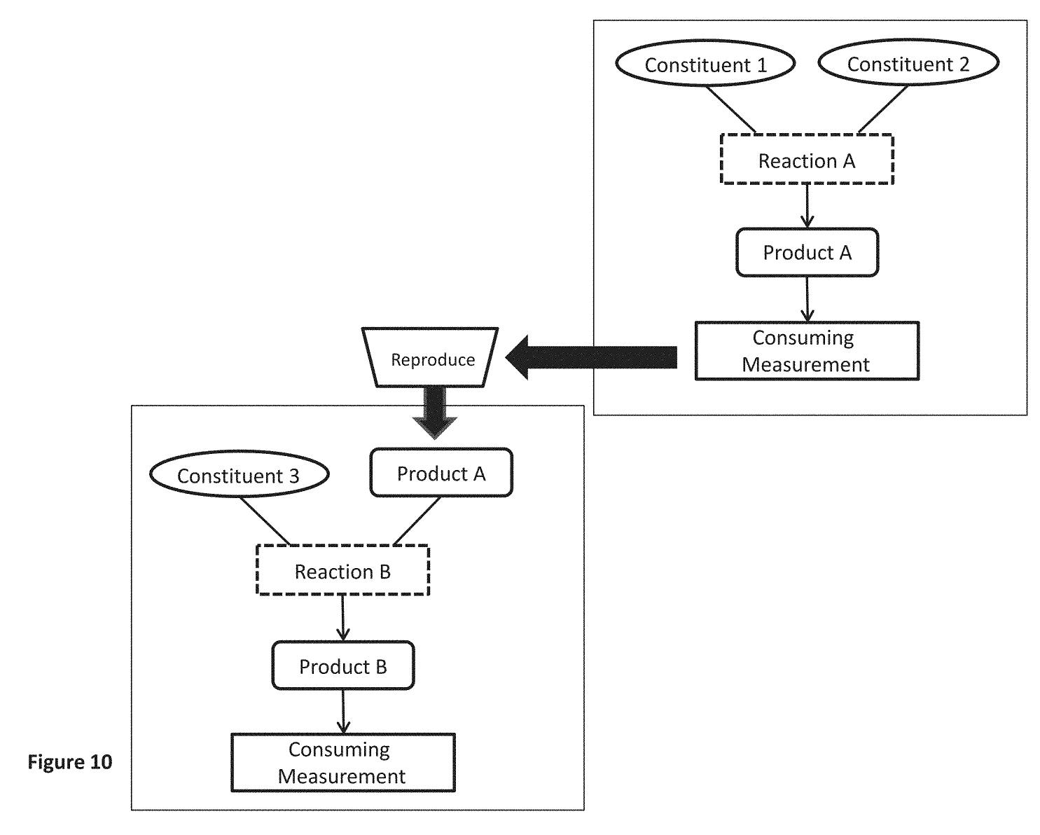

FIG. 8 shows an example of a “blockwise” replica reaction that consumes reaction product B. If knowledge of reaction product A was also needed, a non-consuming measurement would be needed to not affect the overall “blockwise” replica reaction. If this is not possible, the replica reaction of FIG. 8 could be broken into parts, for example the “stepwise” replica reactions depicted in FIGS. 9a and 9b. Here a consuming measurement of reaction product A is made for the “stepwise” replica reaction depicted in FIG. 9a, and controlled introduction of that reaction product A is (then or later) provided by computer control as a constituent material into the “stepwise” replica reaction depicted in FIG. 9b. This is illustrated in FIG. 10. In one example, the “stepwise” replica reactions depicted in FIGS. 9a and 9b can be joined together by a simulation algorithm in emulation-simulation hybrid as defined above.

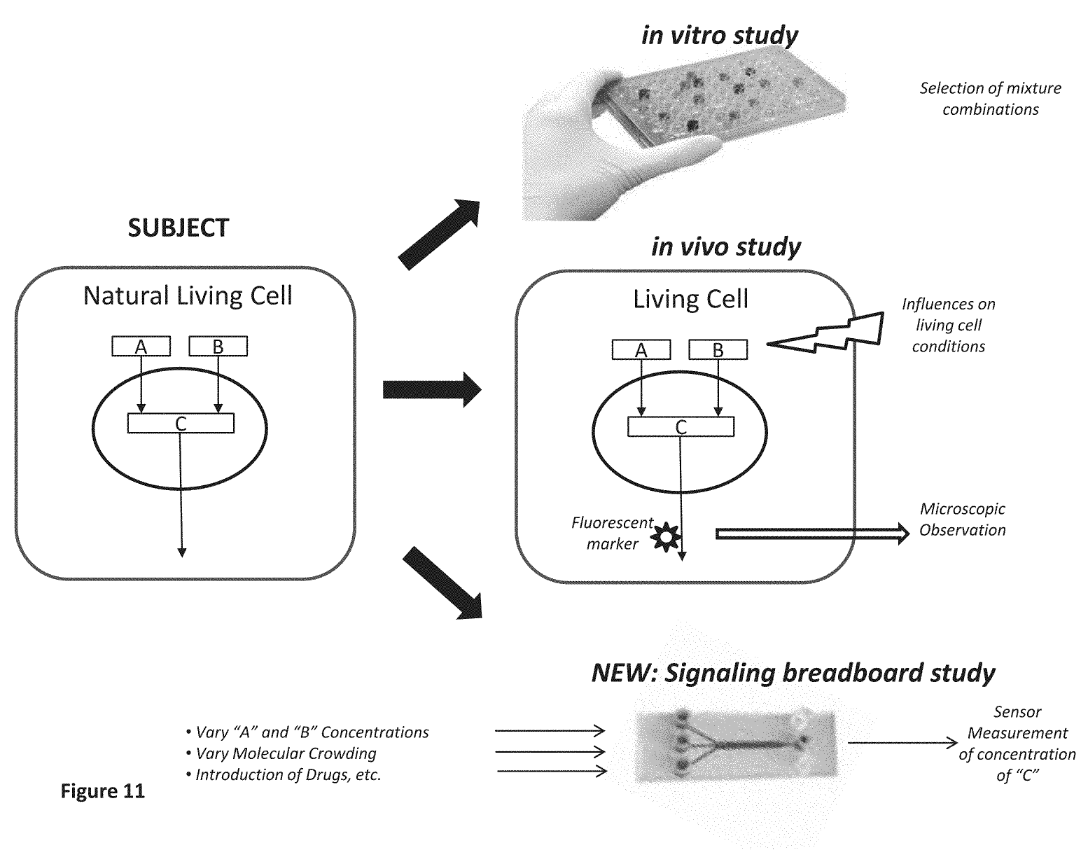

With the invention thus somewhat established, it is possible to compare it somewhat with in vitro and in vivo techniques used in studying the biochemistry and phenomenology of biochemical pathways as suggested in FIG. 11.

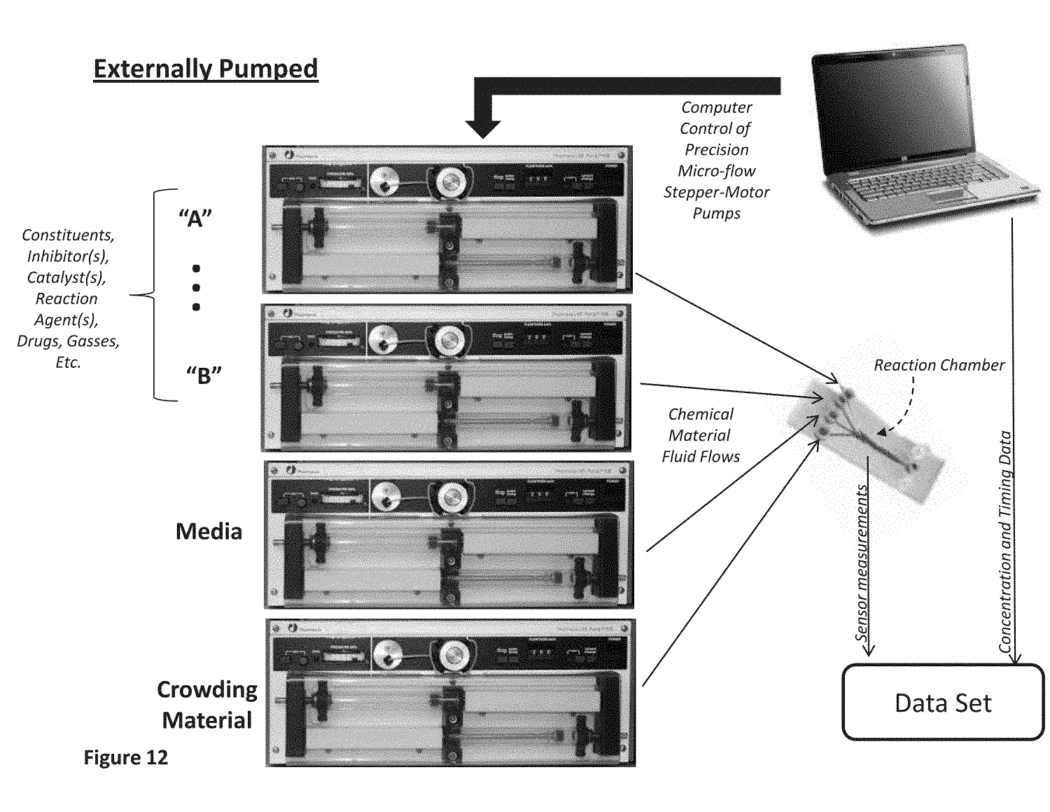

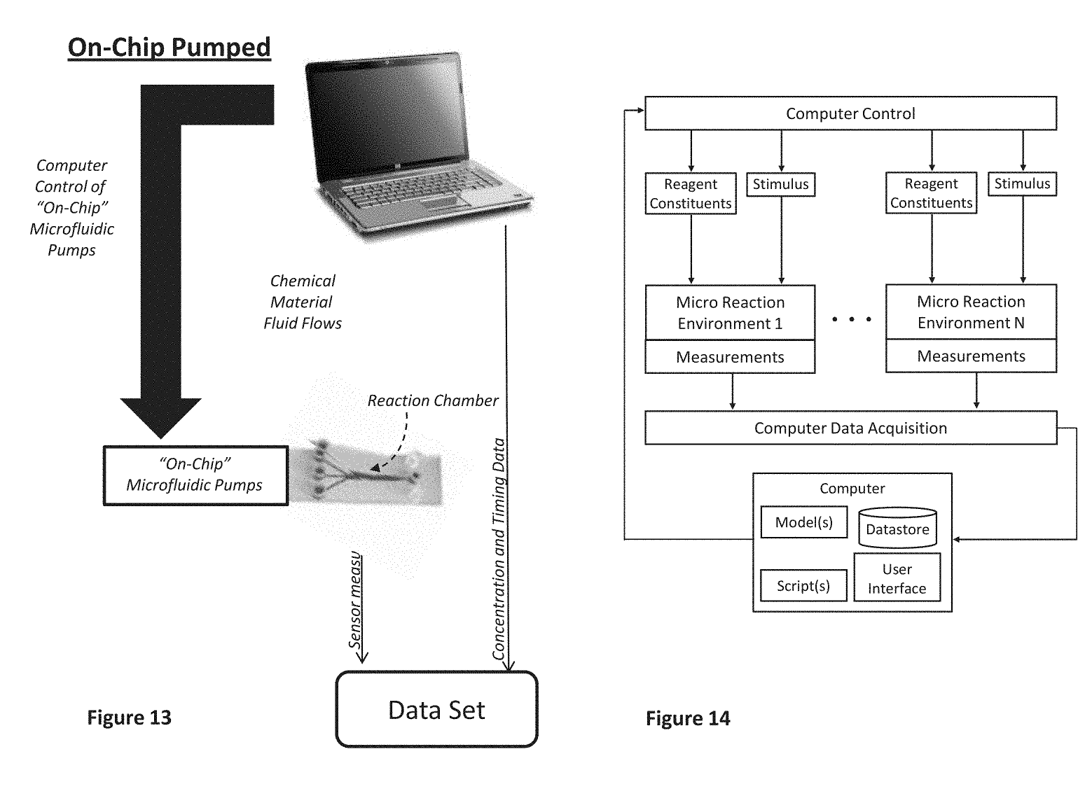

Regarding the controlled application of constituents, FIG. 12 depicts a simple example of a signaling pathway step emulation employing external computer controlled precision micro-flow stepper-motor pumps such as those commonly used in precision liquid chromatography. Alternatively, on-chip microfluidic pumps can be used, for example as suggested in FIG. 13.

Implementing Replicas of Partitioned Biochemical Pathways

Embodiments of the biological signaling breadboard or features therein can comprise one or more microscale or nanoscale chemical reaction environments that can be used to implement a replica partitioned portion of larger biochemical processes and pathways for signaling, metabolism, and gene regulation. For example, such replica partitioned chemical reaction environments can be arranged to:
accept reactants, reagents, and other material passively and/or responsive to computer control via fluidic inputs,
comprise at least one reaction environment,
include or support sensors or internal instrumentation for monitoring one or more of:
   the presence or concentration of chemical/biochemical species,
   the presence and progress of chemical/biochemical processes,
include or support aspects of external instrumentation for monitoring one or more of:
   the presence or concentration of chemical/biochemical species,
   the presence and progress of chemical/biochemical processes,
provide controlled introduction of one or more chemical/biochemical materials,
provide a controlled environment required maintain one or more chemical/biochemical processes,
provide controlled stimulus to initiate or maintain one or more chemical/biochemical processes.

In many embodiments, replica partitioned chemical reaction environments can be arranged to provide outlets for removing the reaction products.

In some embodiments, replica partitioned chemical reaction environments can comprise at least one membrane.

Functionally Interconnecting Replica Partition Chemical Reaction Environments with a Computer and Algorithms for Chemical Reaction Environment Control and Chemical Reaction Environment Measurements

FIG. 14 depicts an example embodiment comprising a plurality of computer-controlled and computer-monitored microscale or nanoscale chemical reaction environments and a computer-controlled arrangement for executing algorithms and interfacing with the plurality of computer-controlled and computer-monitored microscale or nanoscale chemical reaction environments.

Embodiments of the biochemical signaling breadboard can further be configured to interface with a computing system performing one or more of the following functions:
Receive measurement information from the sensors and/or instrumentation associated with each of the one or more microscale or nanoscale chemical reaction environments;
Transmit control information used to control fluidics systems.
Transmit control information used to control the introduction of one or more chemical/biochemical materials into each of the one or more microscale or nanoscale chemical reaction environments;
Transmit control information used to control the stimulus of one or more chemical/biochemical processes into each of the one or more microscale or nanoscale chemical reaction environments;
Execute control algorithms for creating and timing the transmitting of the aforementioned control information;
Execute feedback control algorithms for creating and timing the transmitting of the aforementioned control information responsive to received measurement information;
Execute storage algorithms for at least storing the aforementioned measurement information to create stored measurement information;
Execute retrieval algorithms for at least retrieving the aforementioned stored measurement information;
Execute control algorithms for creating and timing the transmitting of the aforementioned control information responsive to retrieved stored measurement information;
Execute analysis algorithms for at least analyzing the aforementioned measurement information;
Execute storage algorithms for at least storing the aforementioned analysis information to create stored analysis information;
Execute retrieval algorithms for at least retrieving the aforementioned stored analysis information;
Execute script-driven control algorithms for creating and timing the transmitting of the aforementioned control information responsive to retrieved stored analysis information;
Support the use of scripts and script-driven control algorithms;
Provide user interface functions.

Functionally Interconnecting Replica Partition Chemical Reaction Environments Using Computer Algorithms Invoking Chemical Reaction Environment Control Responsive to Chemical Reaction Environment Measurements

In embodiments where there are more than one microscale or nanoscale chemical reaction environments can be configured to comprise linking algorithms or other arrangements wherein received measurement information associated with one of the microscale or nanoscale chemical reaction environments is used for creating and timing the transmitting of control information directed to at least one other of the microscale or nanoscale chemical reaction environments.

In embodiments comprising at least one such linking algorithm, the combination of controlled and (sensor or instrument) monitored microscale or nanoscale chemical reaction environments together with the linking algorithms can be used to emulate a signaling cascade or portion of a signaling network.

In embodiments comprising a plurality of such linking algorithm, the combination of controlled and (sensor or instrument) monitored microscale or nanoscale chemical reaction environments together with the linking algorithms can be used to emulate a larger signaling cascade, portion of a signaling network, or entire signaling network.

In embodiments comprising a plurality of such linking algorithms, and wherein the linking introduces a feedback loop involving two or more of the microscale or nanoscale chemical reaction environments, the combination of controlled and (sensor or instrument) monitored microscale or nanoscale chemical reaction environments together with the linking algorithms can be used to emulate signaling feedback, a signaling cascade with feedback, a portion of a signaling network comprising feedback, or entire signaling network comprising feedback.

In embodiments comprising a plurality of such linking algorithms, and wherein the linking introduces a feedforward path involving two or more of the microscale or nanoscale chemical reaction environments, the combination of controlled and (sensor or instrument) monitored microscale or nanoscale chemical reaction environments together with the linking algorithms can be used to emulate signaling feedforward, a signaling cascade with feedforward, a portion of a signaling network comprising feedforward, or entire signaling network comprising feedforward paths.

In an embodiment, the two partitions depicted in FIG. X4a are implemented with computer-controlled and computer-monitored microscale or nanoscale chemical reaction environments linked by at least one algorithm executing on a computer, and the roles of X, Y, Z, and C are handled by at least one algorithm executing on the computer or a related computer.

In an embodiment, the two partitions depicted in FIG. X4a are implemented with computer-controlled and computer-monitored microscale or nanoscale chemical reaction environments linked by at least one algorithm executing on a computer, and the roles of X, Y, and Z are handled by at least one algorithm executing on the computer or a related computer.

Fluidically Interconnecting Replica Partition Chemical Reaction Environments

Embodiments of the biochemical signaling breadboard can further be configured to include at least one fluidic interconnection between at least two of the microscale or nanoscale chemical reaction environments. In some embodiments the fluidic interconnection is simply gated on and off under computer control. In other embodiments the fluidic interconnection is realized as one possible configuration of a computer-controlled reconfigurable fluidic interconnection network. In some embodiments the computer-controlled reconfigurable fluidic interconnection network can be implemented as, or comprise aspects of, a controllable multichannel microfluidic chemical bus such as that taught in pending U.S. Pat. Nos. 8,032,258 and 8,606,414 and pending U.S. patent application Ser. No. 13/251,288.

In embodiments comprising at least one such fluidic interconnection, the combination of controlled and (sensor or instrument) monitored microscale or nanoscale chemical reaction environments together with the linking algorithms can be used to emulate a signaling cascade or portion of a signaling network.

In embodiments comprising a plurality of such fluidic interconnection, the combination of controlled and (sensor or instrument) monitored microscale or nanoscale chemical reaction environments together with the linking algorithms can be used to emulate a larger signaling cascade, portion of a signaling network, or entire signaling network.

In embodiments comprising a plurality of such fluidic interconnections, and wherein the linking introduces a feedback loop involving two or more of the microscale or nanoscale chemical reaction environments, the combination of controlled and (sensor or instrument) monitored microscale or nanoscale chemical reaction environments together with the linking algorithms can be used to emulate signaling feedback, a signaling cascade with feedback, a portion of a signaling network comprising feedback, or entire signaling network comprising feedback.

In embodiments comprising a plurality of such fluidic interconnections, and wherein the linking introduces a feedforward path involving two or more of the microscale or nanoscale chemical reaction environments, the combination of controlled and (sensor or instrument) monitored microscale or nanoscale chemical reaction environments together with the linking algorithms can be used to emulate signaling feedforward, a signaling cascade with feedforward, a portion of a signaling network comprising feedforward, or entire signaling network comprising feedforward paths.

Anomalous Diffusion Processes Resulting from Molecular Crowding and Confined/Constrained Biochemical Reaction Environments

An important aspect in a faithfully rendered replica chemical reaction environment is provision for the almost universally ignored need for the inclusion of the effects if not accurate emulation molecular crowding. Formulations, models, simulations, and emulations that do not include accurate provisions for molecular crowding provide incorrect molecular-transport statistical thermodynamics and can completely omit reaction processes that naturally occur in living cells. For example, the statistical thermodynamics for molecular-transport without consideration of molecular crowding are those of classical Brownian motion, which in turn provides statistically reproducibility of time-observables, ensemble averaging, long-time convergences, the interchangeable equivalence of time-averaging and ensemble-averaging, and other ergodic and related properties. In contrast, the dense macromolecular environment inside living cells induces molecular crowding that dramatically shifts the statistical thermodynamics for molecular-transport to at least two gross types of “anomalous diffusion” processes that absolutely do not share the many singular privileges of classical Brownian motion. For these, time-averaged observables are not reproducible, time-translation invariance are not respected, molecular-transport is not regularized, diffusion is jumpier and slower, and diffusion exponents differing significantly from the value of 1 provided by classical Brownian motion result, with implications as to whether molecular interactions involving reaction barriers and restricted alignments have sufficient time to occur. Anomalous diffusion processes for molecules traveling throughout the crowded molecular environment of a living cell can be more accuracy modeled with the Continuous Time Random Walk (“CTRW”), a non-ergodic random process demonstrating excellent agreement with a wide range of measurement subjects. Lattice models are employed for modeling diffusion-limited bimolecular reactions where a small number of reactants diffuse in a crowded environment among a much larger number of inert particles; these provide confirming related results from a colloidal physical chemistry viewpoint. Anomalous diffusion processes for molecules confined within a smaller spatial region, for example in the telomeres of a chromosome, a monomer in a polymer chain, or a molecule embedded in a membrane, is the Fractional Brownian Motion (“FBM”) process. In contrast to classical Brownian motion, diffusion exponents for appropriate CTRM models have values of ˜0.7 while diffusion exponents for appropriate FMB models can have value of ˜0.3. Accessible well-written accounts describing these and other recent appreciations of molecular crowding can be found in the article by E. Barkai, Y. Garini, and R. Metzler entitled “Strange Kinetics of Single Molecules in Living Cells” published in Physics Today, Vol. 65 No. 8, August 2012, pp. 29-35 and the chapter by A. Minton and G. Rivas entitled “Biochemical Reactions in the Crowded and Confined Physiological Environment: Physical Chemistry Meets Synthetic Biology” in The Minimal Cell: The Biophysics of Cell Compartment and the Origin of Cell Functionality, P. Luisi and P. Stano (eds.) 2011, ISBN 9048199433, pp. 73-89.

Controlled Molecular Crowding Emulation

Thus not only would a model or simulation that does not account for molecular crowding encounter accuracy shortcomings, but an emulated or other experimental environment for studying the existence of and rate constants for biochemical reactions that occur in cells which does not account for molecular crowding can be expected to have significant, if not profound, accuracy shortcomings.

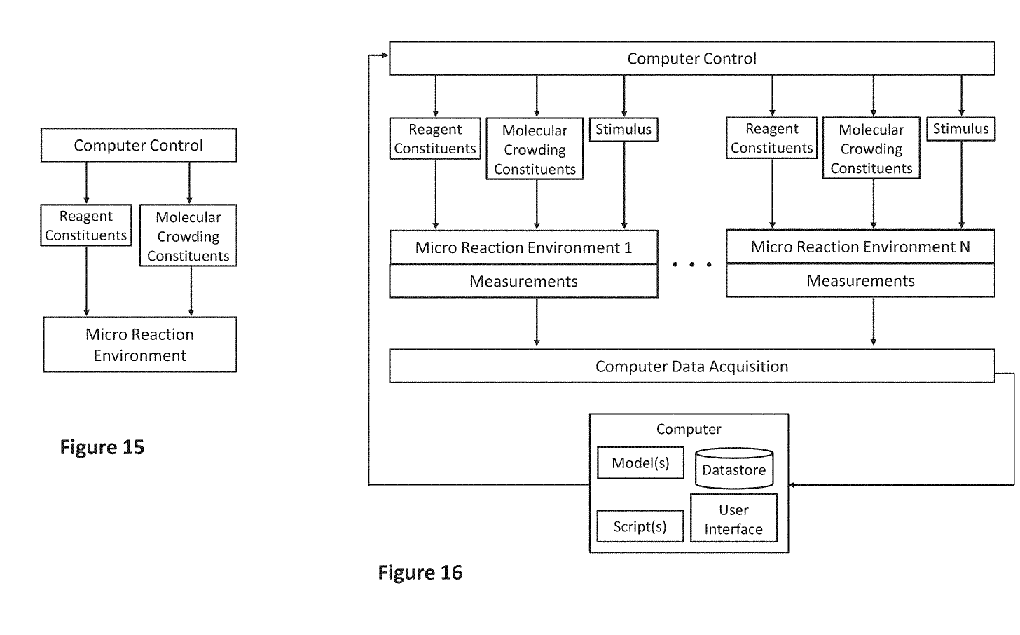

Accordingly, the present application provides for emulating molecular crowding through the controlled introduction of molecular crowding constituents into a monitored microscale or nanoscale chemical reaction environment. Replica partition chemical reaction environments can be arranged to include controlled degrees of substitute or representative molecular crowding. For example, FIG. 15 depicts an example approach to emulating molecular crowding through the controlled introduction of molecular crowding constituents into a monitored microscale or nanoscale chemical reaction environment. As another example, FIG. 16 depicts an example variation on the example embodiment depicted in FIG. 14 that incorporates the approach to emulating molecular crowding depicted in FIG. 15.

Confined and Constrained Reaction Environments for Replica Partition Chemical Reaction Environments

Similarly, not only would a model or simulation that does not account for confined and constrained reaction environments encounter accuracy shortcomings, but an emulated or other experimental environment for studying the existence of and rate constants for biochemical reactions that occur in cells which does not account for confined and constrained reaction environments can be expected to have significant, if not profound, accuracy shortcomings.

Aforementioned examples of confined and constrained reaction environments inside living cells include telomeres of a chromosome, a monomer in a polymer chain, or a molecule embedded in a membrane, but many other examples are comprised in cell organelles. As mentioned earlier, these diffusion processes (exhibiting FBM behavior and having diffusion exponent value of for example ˜0.3) differ profoundly from classical Brownian motion (having diffusion exponent value of 1) and also differ significantly from the types of anomalous diffusion processes for molecules traveling throughout the crowded molecular environment of a living cell (exhibiting CTRW behavior and having diffusion exponent value of for example ˜0.7 and other relevant statistical differences from FBM behavior).

Accordingly, the present application provides for replica partition chemical reaction environments to internally comprise one or more confined or constrained reaction environments for emulation of processes occurring within confined reaction environments inside living cells.

Confined and constrained reaction environments can be applicably implemented in a wide variety of ways including but not restricted to structured polymers, graphene and graphite structures, pillared clays and other types of structured clays, structured synthetic zeolites, inclusion compounds and cavity-containing supra-molecular compounds (for example cyclodextrins, calixarenes, and other organic host lattices), clathrates, liposomes, and various types of self-assembled supra-molecular structures. The present application also provides for the use of ordinary and controlled micelles where practical, advantageous, and feasible.

Controlled Dispensing of Drug Constituents into Replica Partition Chemical Reaction Environments

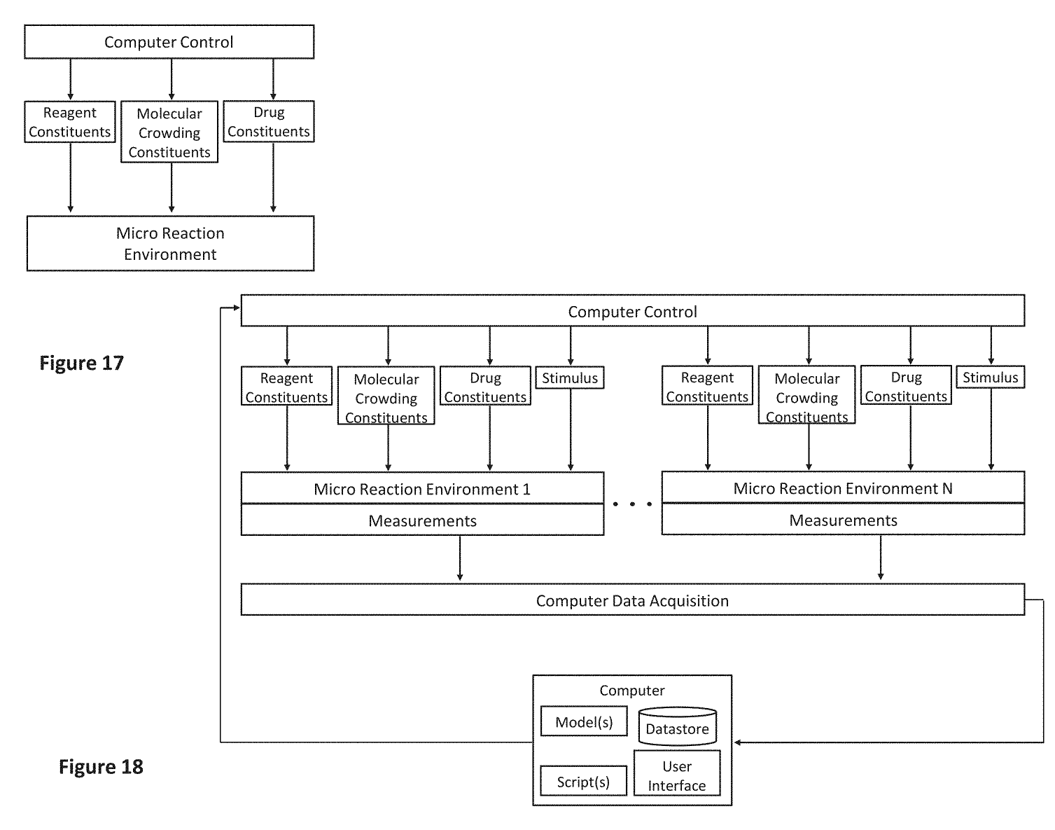

The present application provides for the controlled dispensing of drug constituents into replica partition chemical reaction environments using the same general system architecture. For example, FIG. 17 depicts an example variation on the approach to emulating molecular crowding depicted in FIG. 15 that further incorporates an approach the dispensing of drug constituents. As another example, FIG. 18 depicts an example variation on the example embodiment depicted in FIG. 16 that incorporates the approach to the dispensing of drug constituents depicted in FIG. 17.

Example Measurement Implementations

The present application provides for replica partition chemical reaction environments to internally comprise one or more of monitoring via internal sensors, monitoring via external sensors, and monitoring via other types of instrumentation such a microscopes, NMR systems, lensless optical microscopy and/or optical tomography such as that taught in pending U.S. patent application Ser. Nos. 12/817,107 and 14/105,108 by the present inventor, as well as a wide variety of other types of instruments.

As discussed earlier, internal sensing methods for the sensing of activities in the reaction chambers with internal sensors can be implemented in a wide variety of ways, for example including but not restricted to optical methods involving limited-influence fluorophore and chromophore probes on reactant molecules, bioFET sensing of reaction product concentrations, and a wide range of other current, evolving, emergent, and future techniques.

### Example 1: The Wnt Pathway

The Wnt Signaling Pathway plays an important role in the development of the embryo in many organisms, including humans. During embryonic development, the Wnt pathway plays a key role in body axis formation, especially the anteroposterior and dorsoventral axes. The formation of the body axes during early embryonic development is crucial in determining the overall outcome of the organism. Wnt is also involved in the formation of the dorsal-ventral of the central nervous system through axon guidance. Wnt proteins guide the axons of the spinal cord in an anterior-posterior direction.

The Wnt pathway is also involved in cell differentiation, cell proliferation, and cell migration. In cell differentiation, Wnt signaling promotes differentiation of stem cells into mesoderm and endoderm progenitor cells. These cells are further promoted to differentiate into even more specific cell types. Wnt signaling has been shown to a role in germ cell determination, hair follicle development, lung tissue development, ovary development, nephron development, gut tissue specification, and sex determination. In cell proliferation, the increased levels of β-catenin can initiate transcription of proteins such as cyclin D1, and cmyc, in which both controls the G1 to S phase in the cell cycle. During S phase, DNA replicates and leads to mitosis, which is responsible for cell proliferation. In cell migration, particularly during convergent extension, Wnt signaling aids to mediate the establishment of body axes, tissue formation, limb induction, and other processes during embryonic development.

The canonical Wnt signaling plays a role in the development of benign and malignant breast tumors. It was indicated due to the high levels of β-catenin in the nucleus and cytoplasm. Increased β-catenin is strongly correlated with poor prognosis in breast cancer patients. The over accumulation of β-catenin may be due to mutations in β-catenin, deficiencies in the destruction complex, overexpression of Wnt ligands, or loss of inhibitors.

Wnt signaling can also be involved in type II diabetes, due to its involvement in insulin sensitivity. Overexpression of Wnt5b could lead to increased rate of type II diabetes due to its involvement in adipogenesis, and since type II diabetes has high co-morbidity with obesity. Wnt signaling also activates mitochondrial biogenesis, which leads to an increase production of reactive oxygen species, known to induce DNA and cellular damage. This can cause the development of acute hepatic insulin resistance.

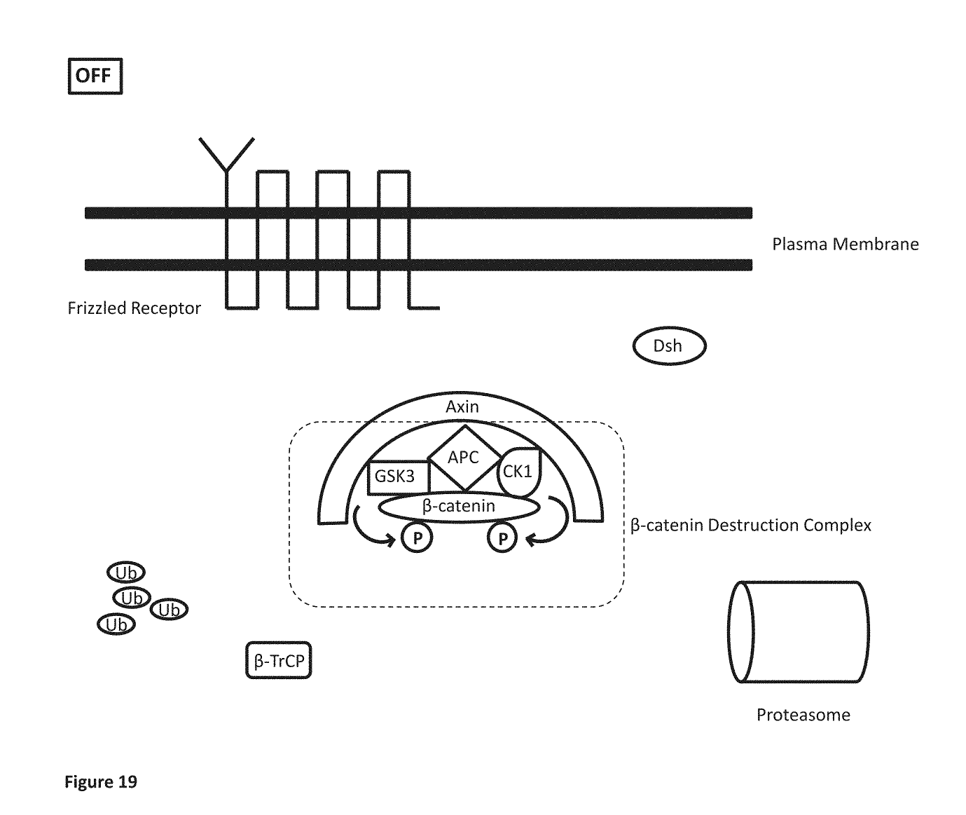

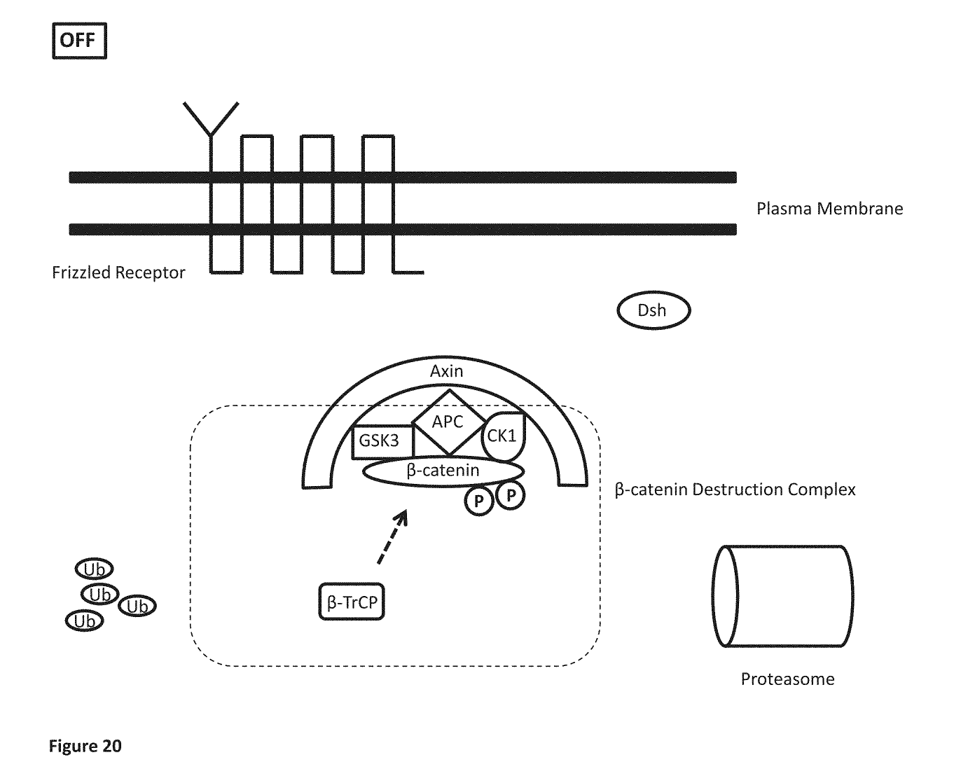

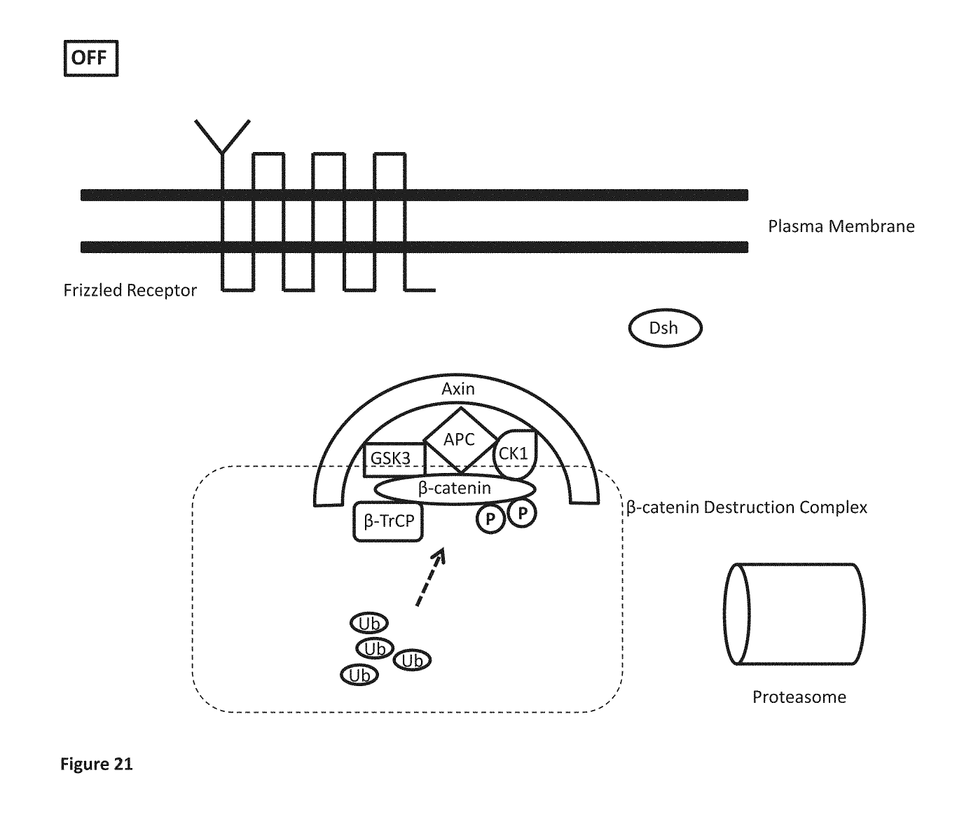

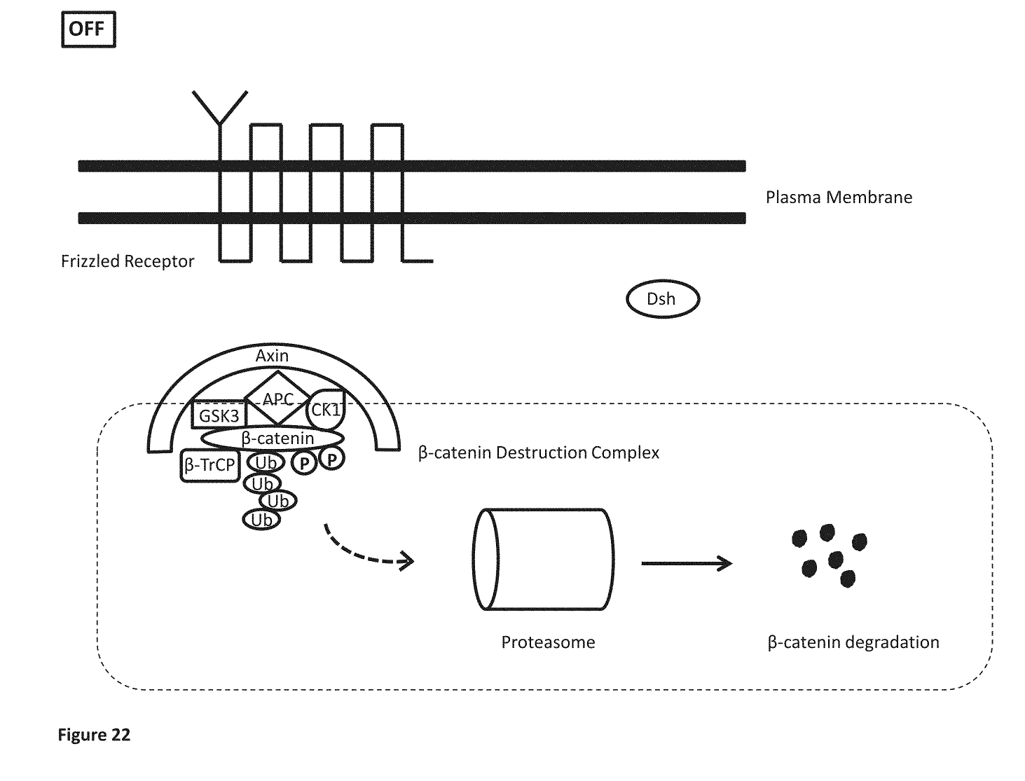

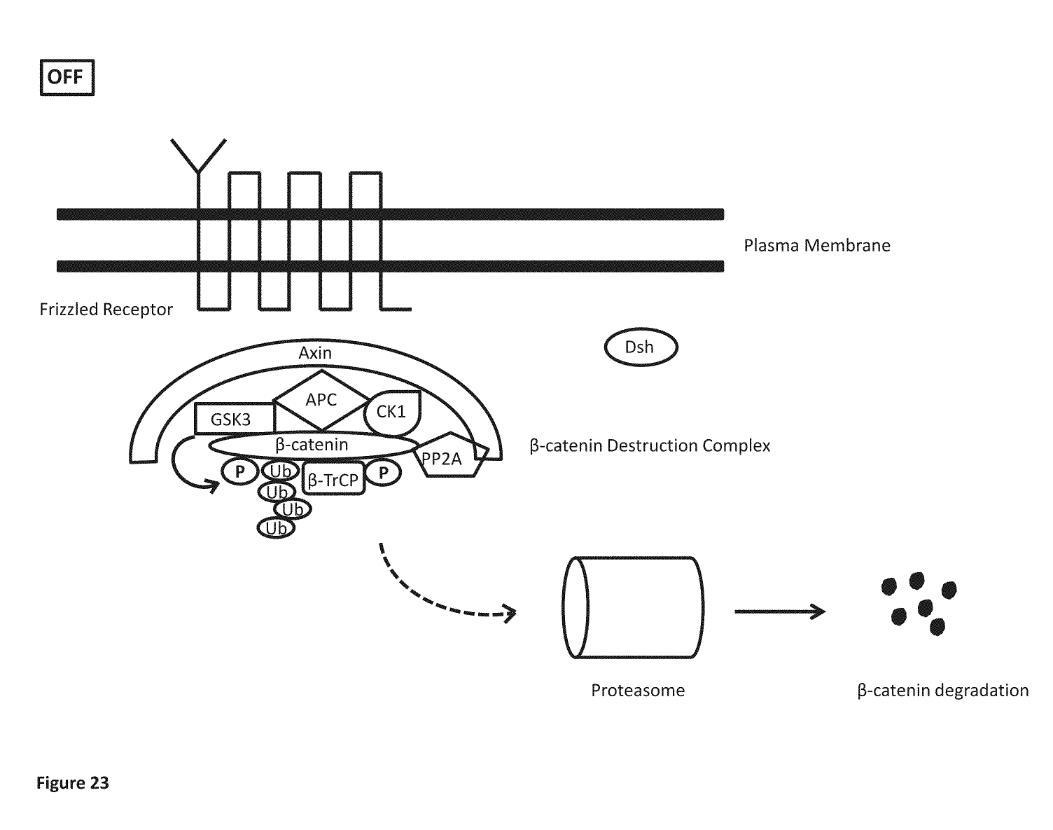

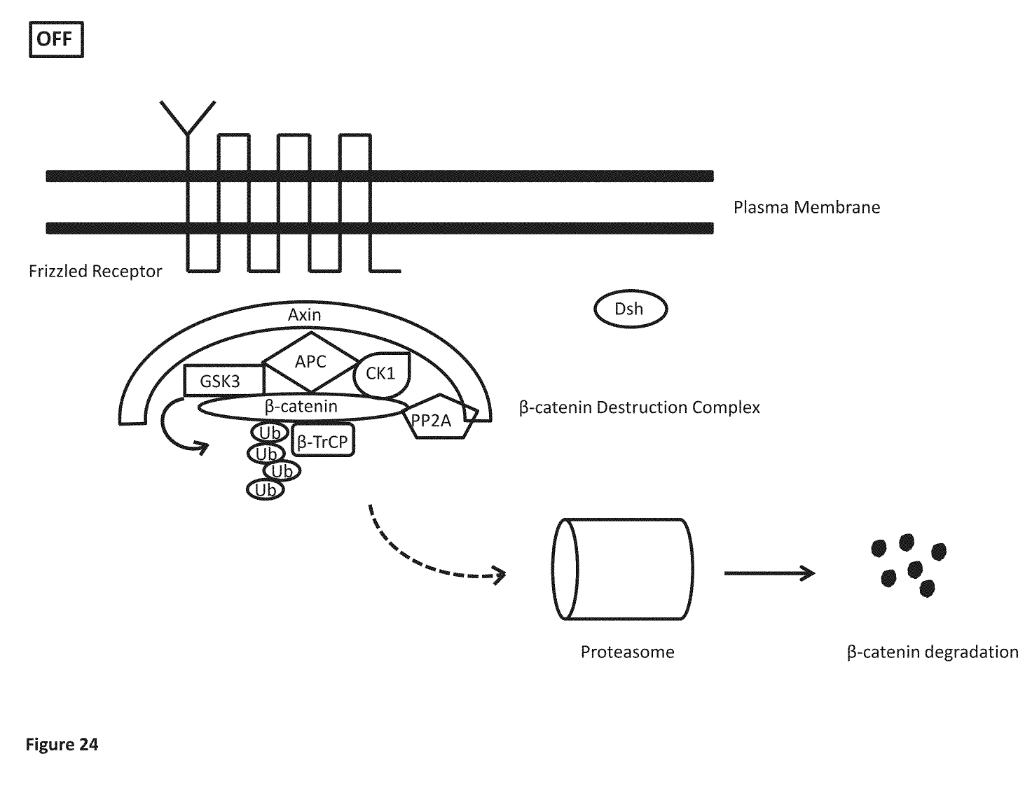

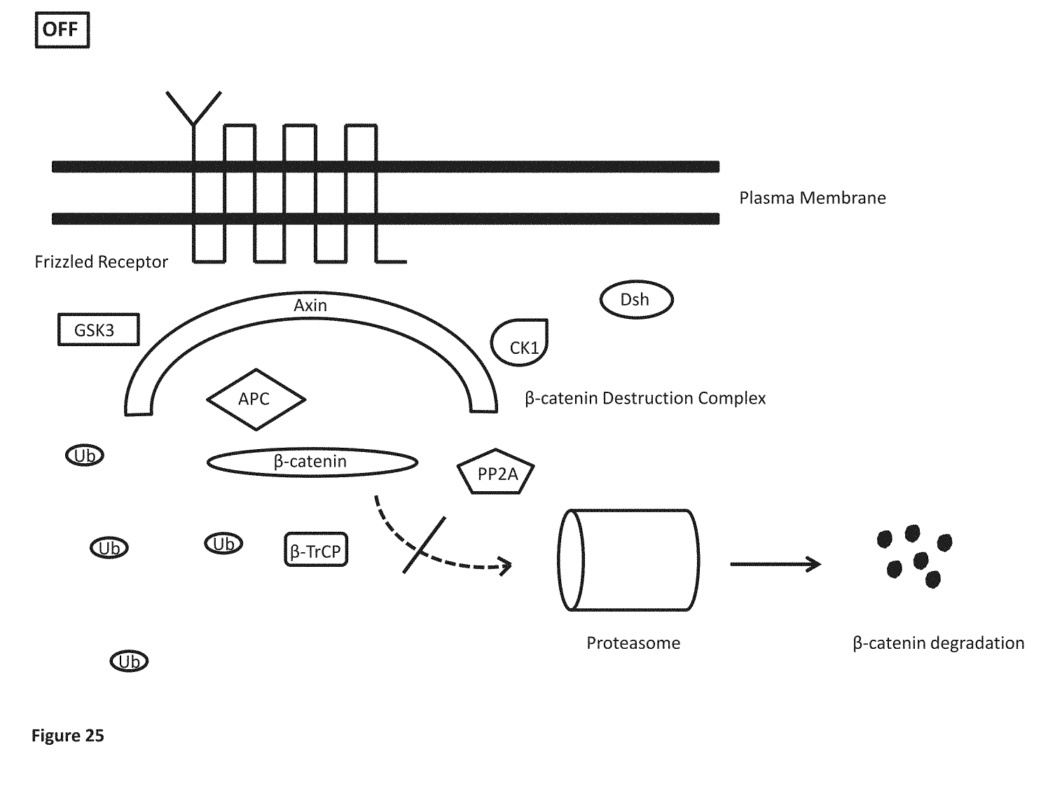

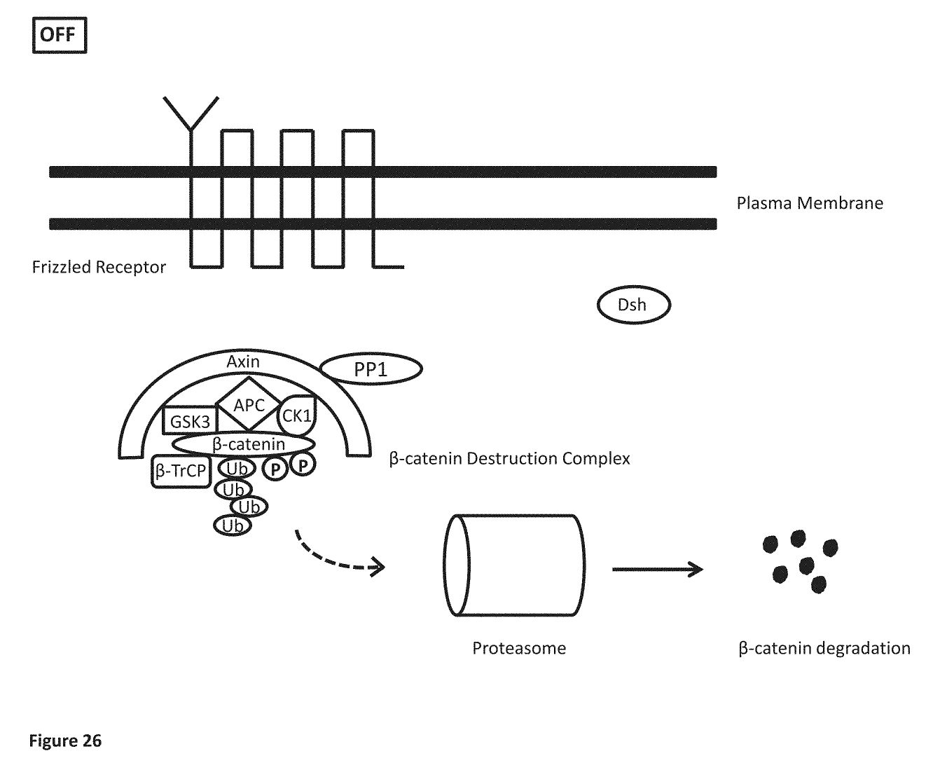

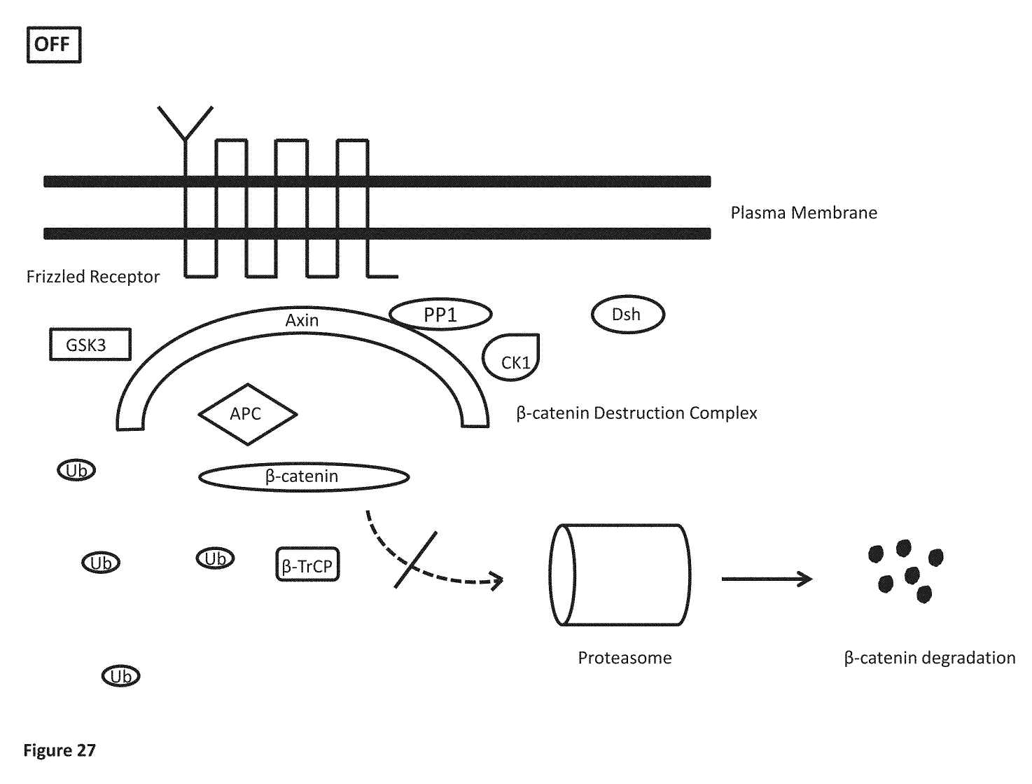

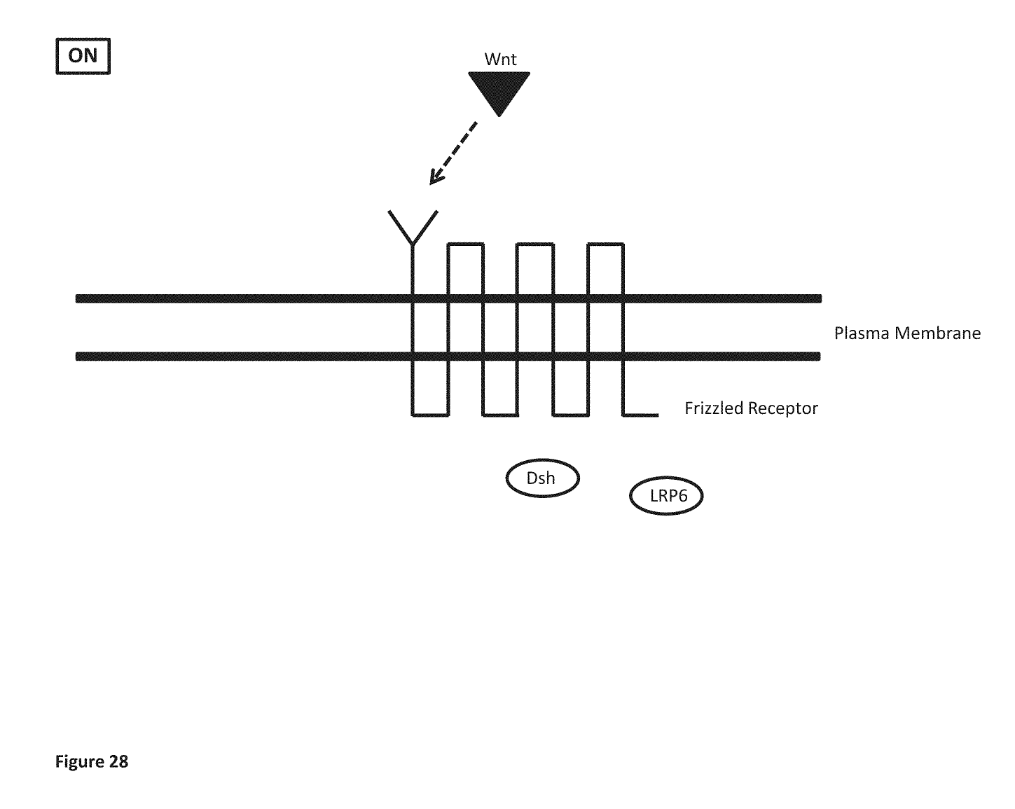

FIG. 19 through FIG. 34 and the following tables provide example sequential step-by-step breakdown of an example contemporary understanding of the Wnt pathway. Each step, represented by a table row and/or one of these figures, represents an example reaction step and is an example candidate for a replica reaction in the context of present invention and extensions of the present invention. As described earlier, the replica reactions can be implemented by embodiments of the present invention or extensions of the present invention in isolation (“stepwise”), and groups of contiguous steps can be implemented by embodiments of the present invention or extensions of the present invention together in a block of “one-pot” reactions (“blockwise”), the block representing a multiple-step segment or uninterrupted subset of the pathway. Example “stoichiometric” steps suitable for execution in the present invention are called out in the 4th column of the tables. The 6th column of the table identifies example correspondences between the example contemporary understanding of the Wnt pathway represented by the figures and example steps of the contemporary understanding of the Wnt pathway represented in of the tables that follow. The first table is directed to the “off” case

| | | Type(s) of | Classes of | | Associated |
|---|---|---|---|---|---|
| Step | Description | Event | events | Constituents | FIGS. |
| 1 | Off- A complex of | Phosphorylation | Stoichiometric | β-catenin, | FIG. 19 |
| | Axin, APC, GSK3, and | | | destruction | |
| | CK1 promotes β- | | | complex, | |
| | catenin | | | GSK3, | |
| | phosphorylation for | | | CK1-α | |
| | degradation (by GSK3 | | | | |
| | and CK1-α). | | | | |
| 2 | β-catenin | Binding | Stoichiometric | β-catenin, | FIG. 20 |
| | phosphorylation | | | β-Trcp | |
| | creates a binding site | | | | |
| | for the E3 ubiquitin | | | | |
| | ligase β-Trcp, inducing | | | | |
| | β-catenin ubiquination | | | | |
| | & degradation. | | | | |
| 3 | The binding of β-Trcp | Ubiquitination | Stoichiometric | β-catenin, | FIG. 21 |
| | subsequently attaches | | | β-Trcp, | |
| | polyubiquitin chains | | | ubiquitin | |
| | onto β-catenin. | | | | |
| 4 | Ubiquitinated | Degradation | Stoichiometric | β-catenin, | FIG. 22 |
| | β-catenin is bound and | | | β-Trcp, | |
| | destroyed by a | | | ubiquitin, | |
| | proteasome promoting | | | proteasome | |
| | proteasomal | | | | |
| | degradation of β-catenin. | | | | |
| 5 | PP2A | Enhancement | Membrane | PP2A, | FIG. 23 |
| | dephosphorylates β- | | Transduction | β-catenin, APC | |
| | catenin, reducing β- | | | | |
| | catenin degradation, | | | | |
| | but APC prevents | | | | |
| | PP2A | | | | |
| | dephosphorylation of | | | | |
| | β-catenin, thus | | | | |
| | enhancing β-catenin | | | | |
| | phosphorylation and | | | | |
| | degradation. | | | | |
| 6 | PP1 dephosphorylates | Dephoshporylation | Membrane | PP1, Axin, | |
| | Axin to antagonize | | Transduction | CK1, GSK3, | |
| | CK1 phosphorylation. | | Stoichiometric | destruction | |
| | | | | complex. | |
| 7 | Axin | Dissociation | Intracellular | PP1, Axin, | |
| | dephosphorylation | | Transport | CK1, GSK3, | |
| | negatively regulates | | Stoichiometric | destruction | |
| | GSK3-Axin binding | | | complex. | |
| | resulting in complex | | | | |
| | disassembly. | | | | |

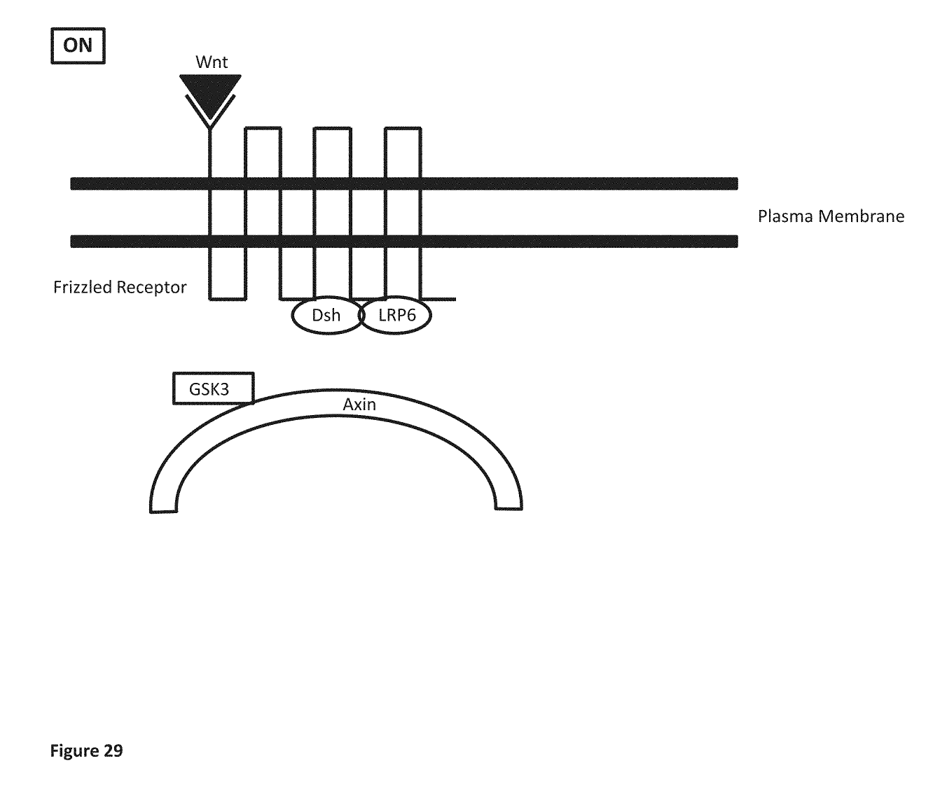

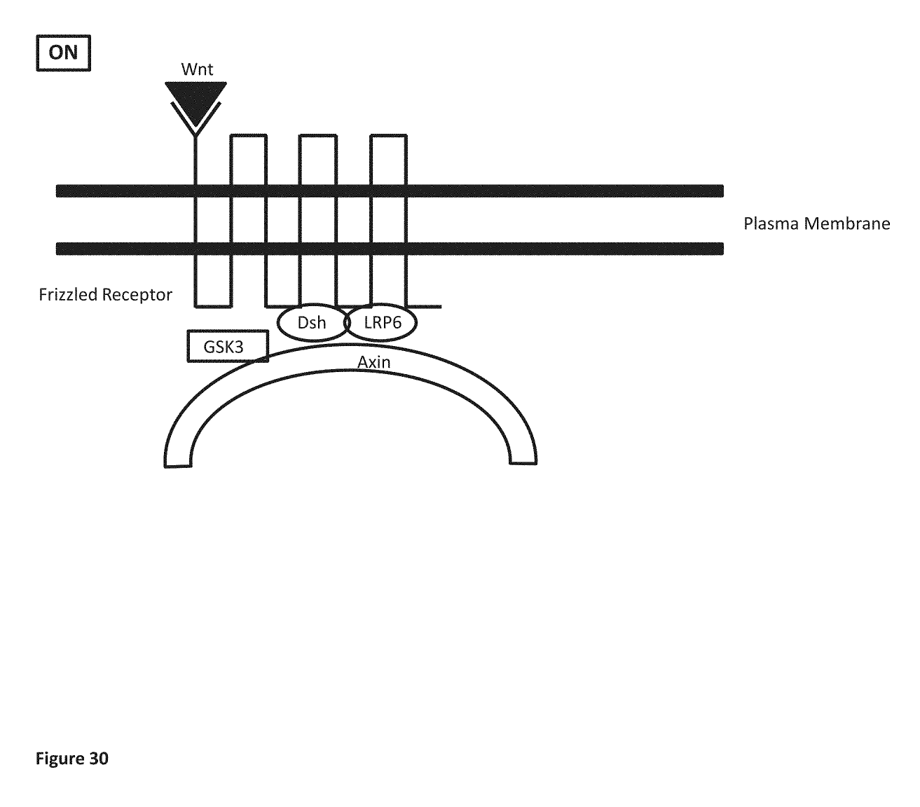

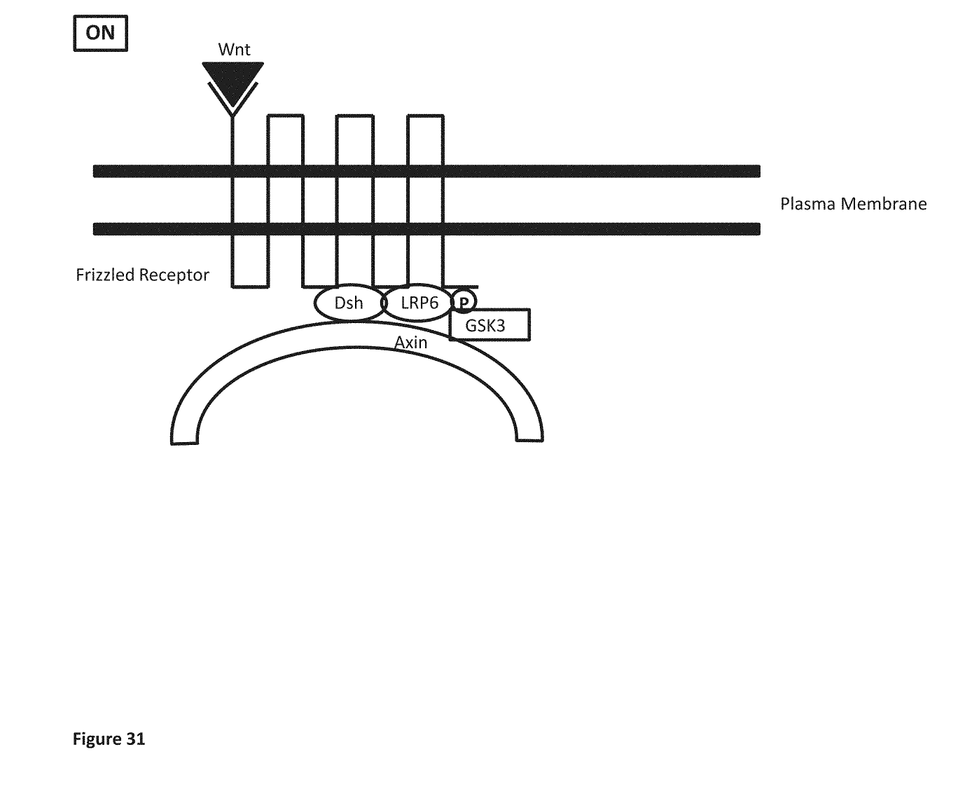

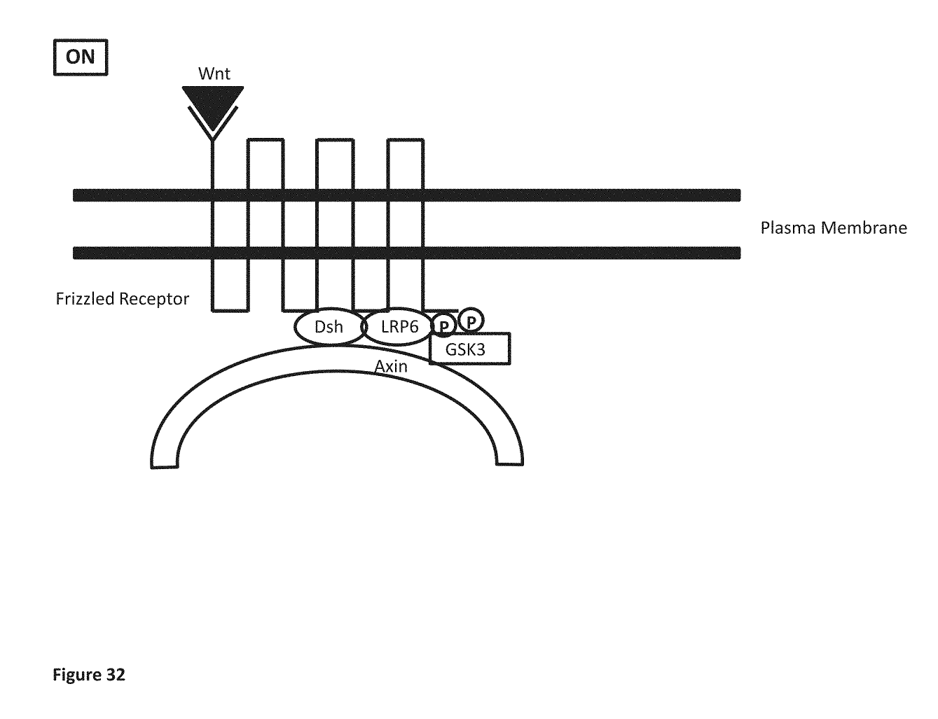

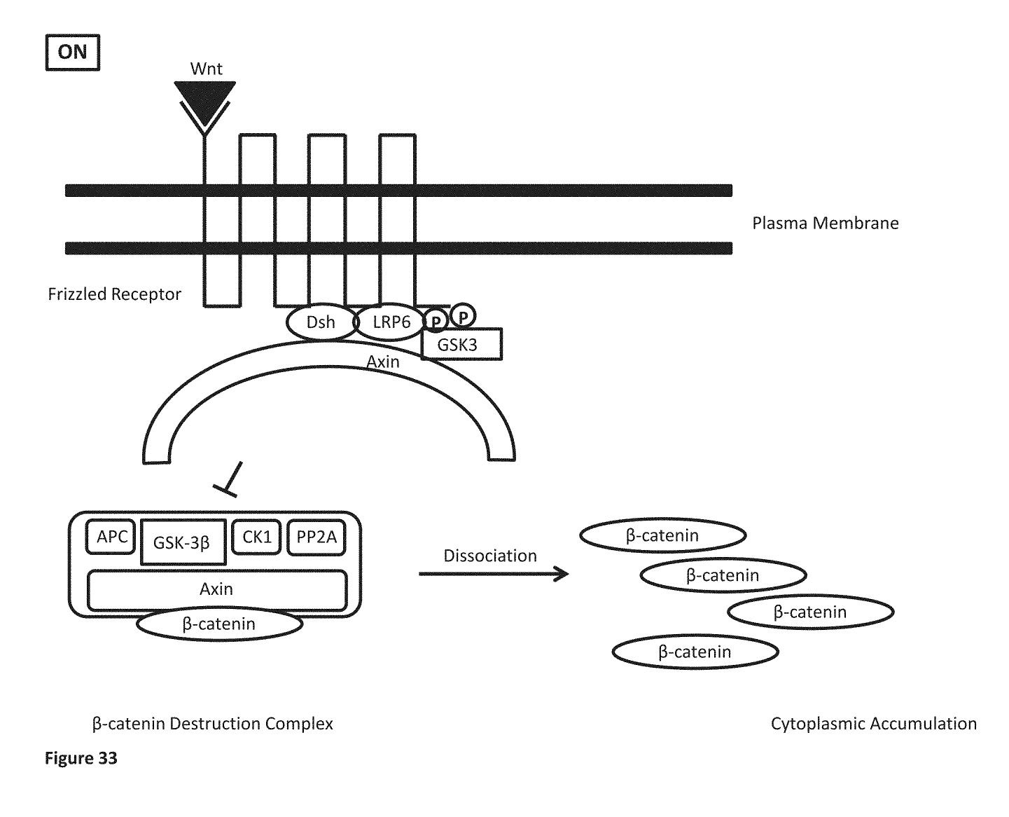

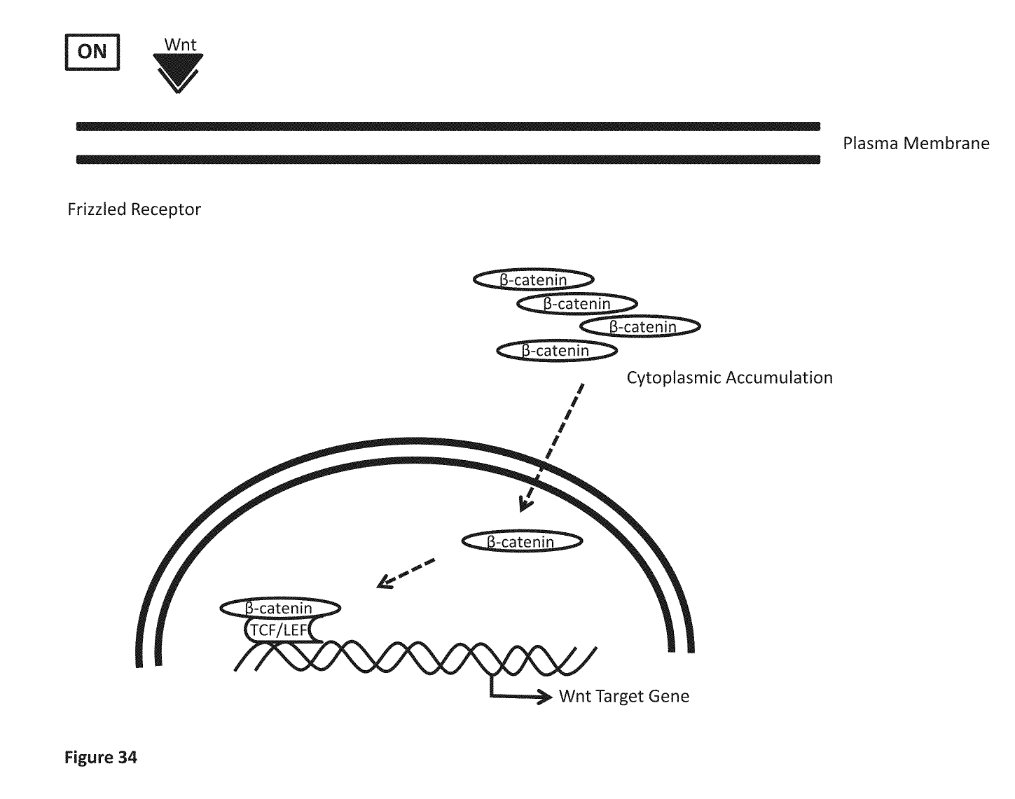

while the following table describes the “on” case.

| | | Type(s) of | Classes of | | Associated |
|---|---|---|---|---|---|
| Step | Description | Event | events | Constituents | FIGS. |
| 1 | On-Wnt protein binds | Binding | Intracellular | Wnt, Frizzled, | |
| | the N-terminal of a | | Transport | Dsh/Dvl | |
| | Frizzled receptor, which | | Stoichiometric | | |
| | is usually linked to | | | | |
| | Dsh/Dvl. | | | | |
| 2 | Wnt binds Fz-Dvl & | Binding | Stoichiometric | Wnt, Fz-Dvl, | FIG. 29 |
| | LRP6 (or ERP5) to | | | LRP6/5, Wnt- | |
| | become the Wnt-Fz- | | | Fz-Dvl-LRP | |
| | Dvl-LRP complex. | | | | |
| 3 | Dvl recruits Axin- | Phosphorylation | Gene | Dvl, Axin- | FIG. 30 |
| | GSK3, resulting in the | | Expression | GSK3, LRP6 | |
| | phosphorylation of | | | | |
| | LRP6 by GSK3 | | | | |
| 4 | The Phosphorylated | Amplification | Intracellular | p-LRP6, Axin | FIG. 31 |
| | LRP6 recruits Axin & a | Phosphorylation | Transport | | |
| | positive feedback loop | | | | |
| | amplifies the | | | | |
| | phosphorylation of | | | | |
| | LRP6 | | | | |
| 5 | Phosphorylated LRP6 | Inhibition | Stoichiometric | P-LRP6, | FIG. 32 |
| | cytoplasmic domain can | | | GSK3, | |
| | directly inhibit GSK3 | | | β -catenin | |
| | phosphorylation of β - | | | | |
| | catenin. | | | | |
| 6 | Overexpression of | Degradation | Stoichiometric | Wnt receptors, | FIG. 33 |
| | activated Wnt receptors | Dissociation | Gene expression | recombinant | |
| | or recombinant Dsh/Dvl | | | Dsh/Dvl, | |
| | can lead to Axin | | | Axin, | |
| | degradation and | | | destruction | |
| | dissociation of the | | | complex | |
| | destruction complex | | | | |
| | made up of the proteins: | | | | |
| | Axin, APC, PP2A, | | | | |
| | CK1a, GSK-3 & β - | | | | |
| | catenin. | | | | |
| 7 | The dissociation of the | Accumulation | Stoichiometric | Destruction | FIG. 33 |
| | destruction complex | | | complex, | |
| | allows β-catenin to | | | β-catenin | |
| | accumulate in the | | | | |
| | cytoplasm. | | | | |
| 8 | Normally, TCF | Translocation | Stoichiometric | β-catenin, | FIG. 34 |
| | interacts with Groucho | Binding | | TCF/LEF, | |
| | to promote histone | | | co-activators | |
| | deacetylation & | | | | |
| | chromation compaction. | | | | |
| | β-catenin replaces | | | | |
| | Groucho, translocating | | | | |
| | into the nucleus to bind | | | | |
| | with TCF/LEF to form | | | | |
| | a complex, recruiting | | | | |
| | other co-activators of | | | | |
| | gene activation. | | | | |
| 9 | TCF/LEF, in addition to | Activation | Stoichiometric | TCF/LEF, | FIG. 34 |
| | co-activators, activates | Transcription | | co-activators, | |
| | RNA polymerase, | | | RNA | |
| | inducing gene | | | polymerase | |
| | transcription. | | | | |

The 5th column of the above table lists constituents of the pathway step described in that row of the table. These constituents would be used in a replica reaction executed by the present invention corresponding to the pathway step described in the associated row of the above table.

The named constituents involved the example contemporary understanding of the Wnt pathway represented by the above table can be produced in a laboratory or in some cases are commercially available or can be produced commercially in the future for use in the present invention and extensions of the present invention. As an example, the table below lists example sources for the constituents identified in the table above.

| Constituents | Description |
|---|---|
| Wnt | The Wnt protein mentioned above, which initiates the Wnt |
| | Signaling Pathway can be obtained from a supplier through this source: |
| | http://www.rndsystems.com/molecule_group.aspx?g=456&r=2&g2=484 |
| | (Visited on Sep. 7, 2014) |
| Frizzled/LPR | The Frizzled, LPR, and Dsh/Dvl proteins bound by the Wnt |
| | protein to form a complex can be obtained from a supplier through these sources: |
| | http://www.rndsystems.com/molecule_group.aspx?g=1920&r=2&g2=484 |
| | (Visited on September 7, 2014) |
| | http://www.rndsystems.com/molecule_group.aspx?g=1922&r=2&g2=484 |
| | (Visited on Sep. 7, 2014) |
| Axin | The following proteins that make up the destruction complex: |
| GSK-3 | APC, PP2, CK1a, GSK-3, & β-catenin, Axin protein that |
| B-catenin | forms the Axin-GSK3 complex can be obtained from a supplier through this source: |
| Complex(APC, | http://www.rndsystems.com/molecule_group.aspx?g=1922&r=2&g2=484 |
| PP2, CK1a, GSK3, | (Visited on Sep. 7, 2014) |
| β -catenin) | |
| TCF | The TCF protein, which activates RNA polymerase, inducing |
| | gene transcription can be obtained from a supplier through this source: |
| | http://www.rndsystems.com/molecule_group.aspx?g=1922&r=2&g2=484 |
| | (Visited on Sep. 7, 2014) |

### Example 2: The Hedgehog Pathway

The Sonic Hedgehog signaling pathway plays a crucial role during development of the vertebrate limb. Sonic Hedgehog signaling promotes proliferation of adult stem cells from tissues, including hematopoietic cells, mammary, and neural stem cells. Mutation or disruption of hedgehog signaling during embryonic development can lead to severe developmental abnormalities, such as Holoprosencephaly, and Cyclopia.

Hedgehog pathway activation has been indicated and implicated in the development of cancers in organs, including the brain, lungs, mammary gland, prostate and skin. The most common form of cancer, Basal cell carcinoma, has the closest association with hedgehog signaling. Patients with this form of cancer have been identified to have mutations in Patched and Smoothened. Unusual activation of the hedgehog pathway leads to development of cancer through transformation of adult stem cells into cancer stem cells, which forms tumors.

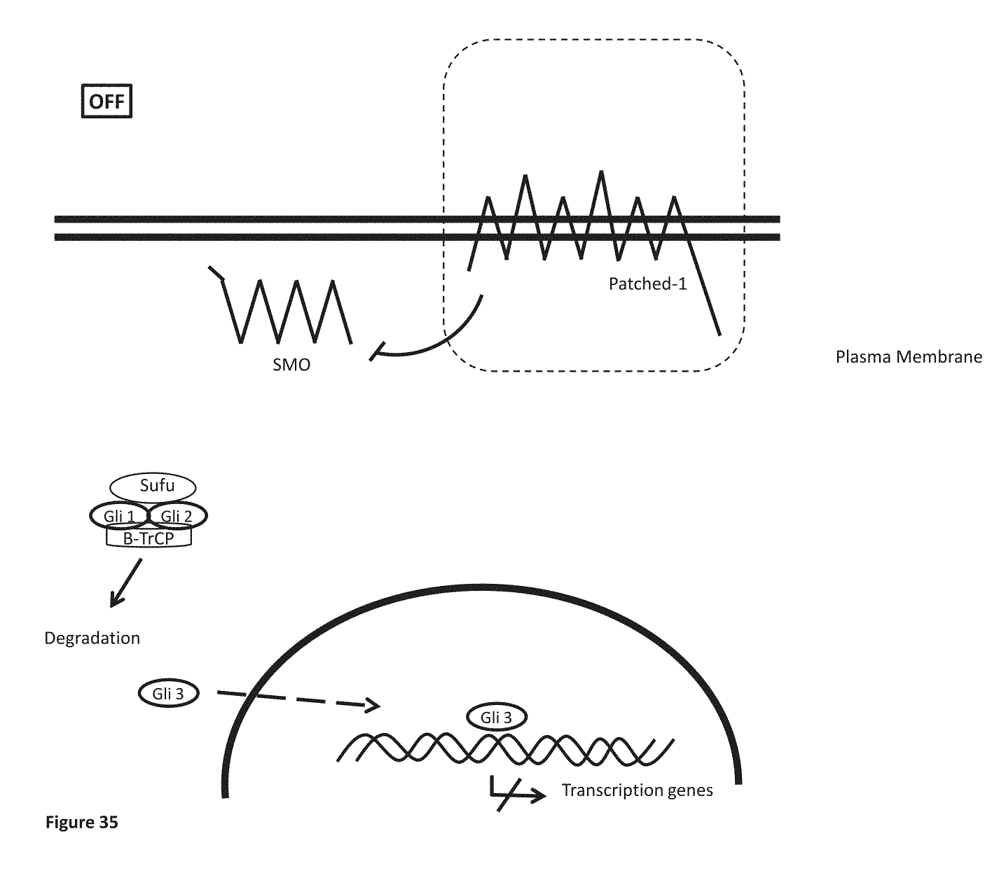

FIG. 35 through FIG. 40 and the following tables provide example sequential step-by-step breakdown of an example contemporary understanding of the Hedgehog pathway. Each step, represented by a table row and/or one of these figures, represents an example reaction step and is an example candidate for a replica reaction in the context of present invention and extensions of the present invention. As described earlier, the replica reactions can be implemented by embodiments of the present invention or extensions of the present invention in isolation (“stepwise”), and groups of contiguous steps can be implemented by embodiments of the present invention or extensions of the present invention together in a block of “one-pot” reactions (“blockwise”), the block representing a multiple-step segment or uninterrupted subset of the pathway. Example “stoichiometric” steps suitable for execution in the present invention are called out in the 4th column of the tables. The 6th column of the table identifies example correspondences between the example contemporary understanding of the Hedgehog pathway represented by the figures and the example steps of represented in of the table. The first table is directed to the “off” case

| | | Type(s) of | Classes of | | Associated |
|---|---|---|---|---|---|
| Step | Description | Event | events | Constituents | FIGS. |
| 1 | Off- Patched-1 interacts | Inhibition | Stoichiometric | Patched-1, | FIG. 35 |
| | with and inhibits SMO. | | | SMO, | |
| 2 | Inactive form of SMO is | Inhibition | Stoichiometric | Inactive form | FIG. 35 |
| | unable to inhibit sufu. | | | SMO, sufu | |
| 3 | Sufu induces | Degradation | Stoichiometric | Sufu, Gli1, Gli2 | FIG. 35 |
| | degradation of Gli | generation | | and Gli3 | |
| | activator (Gli1 and Gli2 | | | | |
| | in mammals) and | | | | |
| | generation of repressor - | | | | |
| | Gli | | | | |

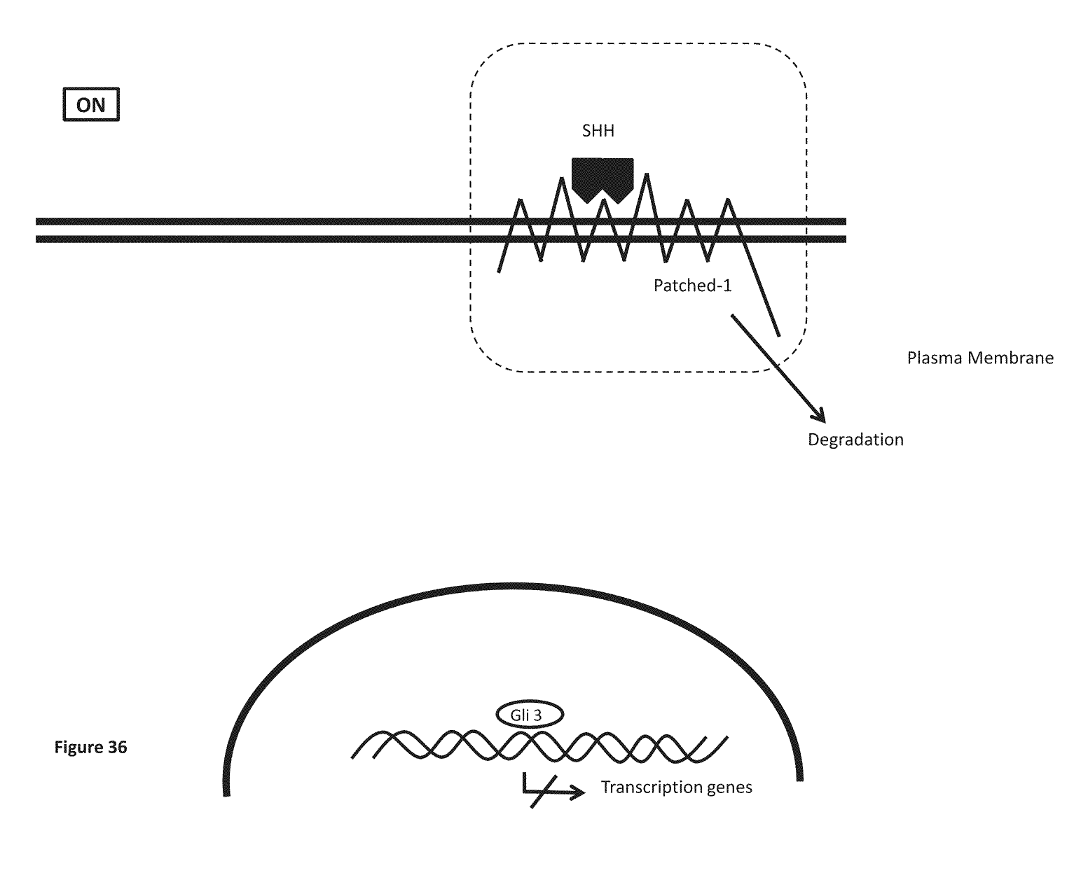

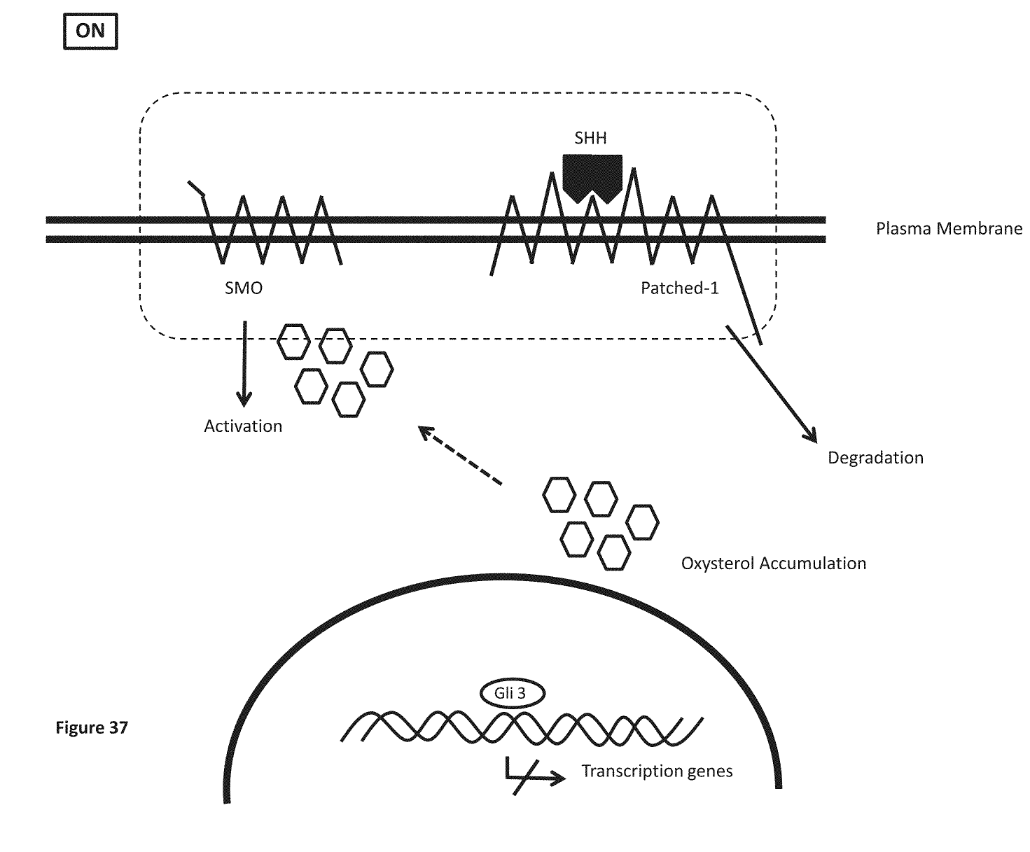

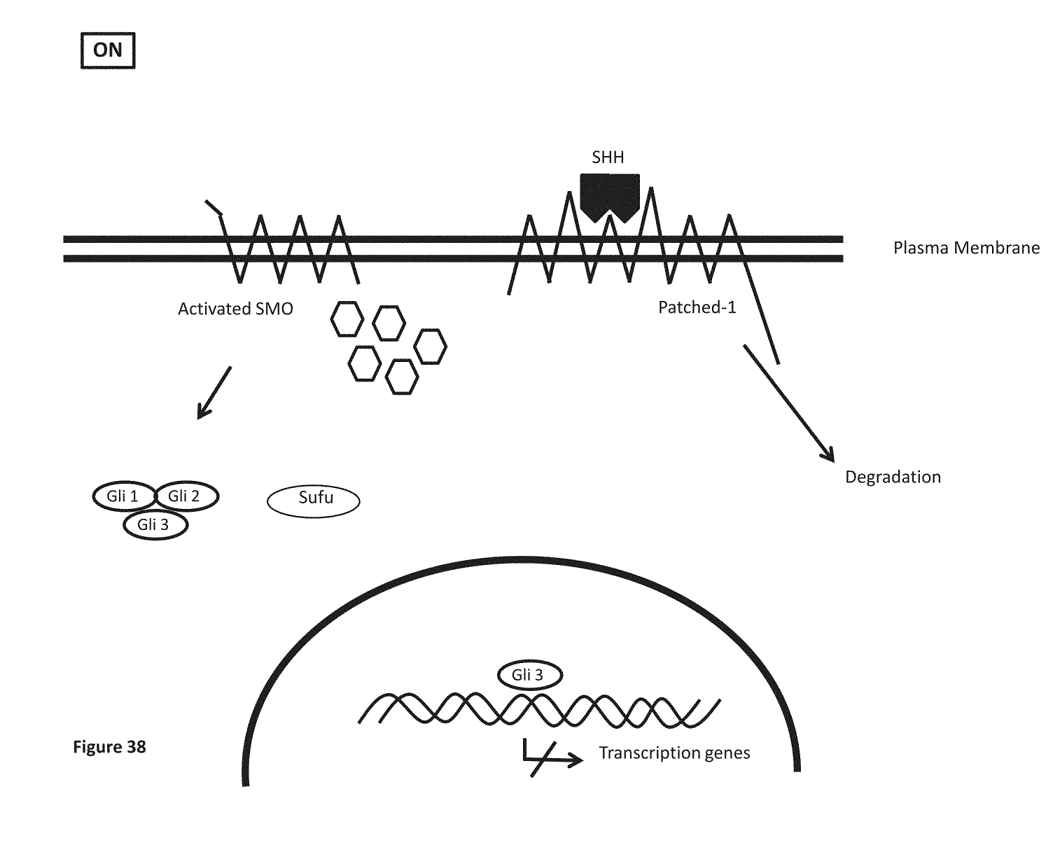

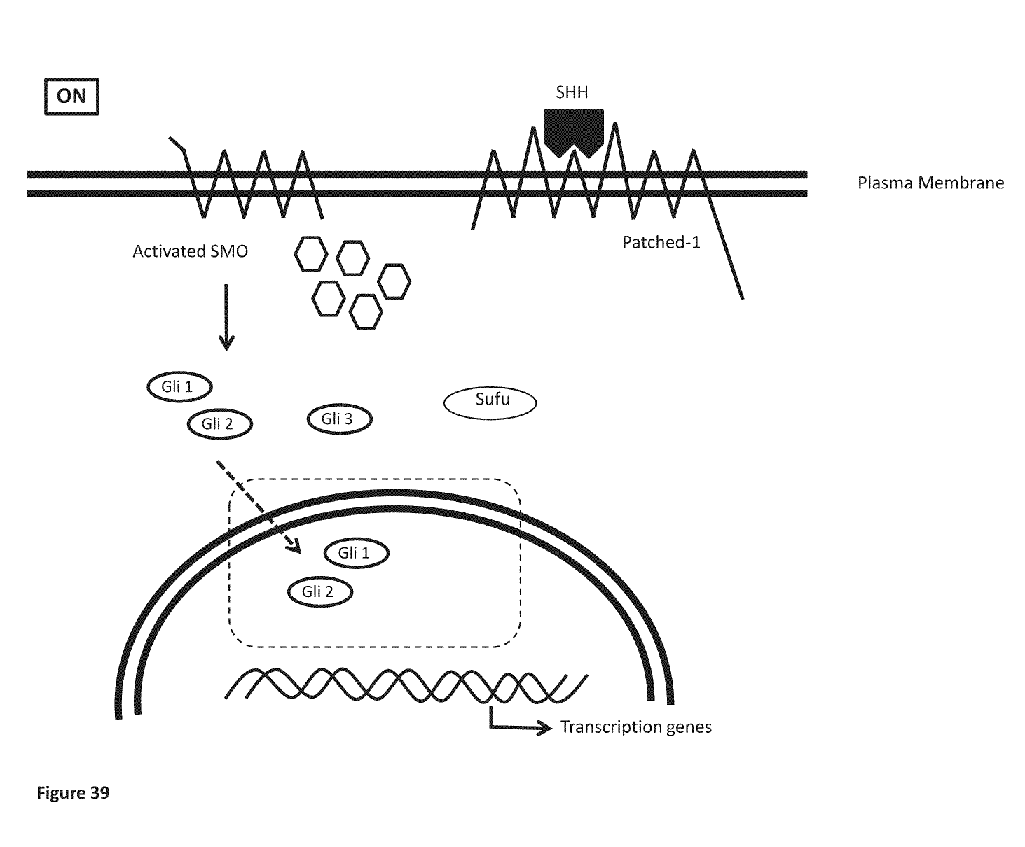

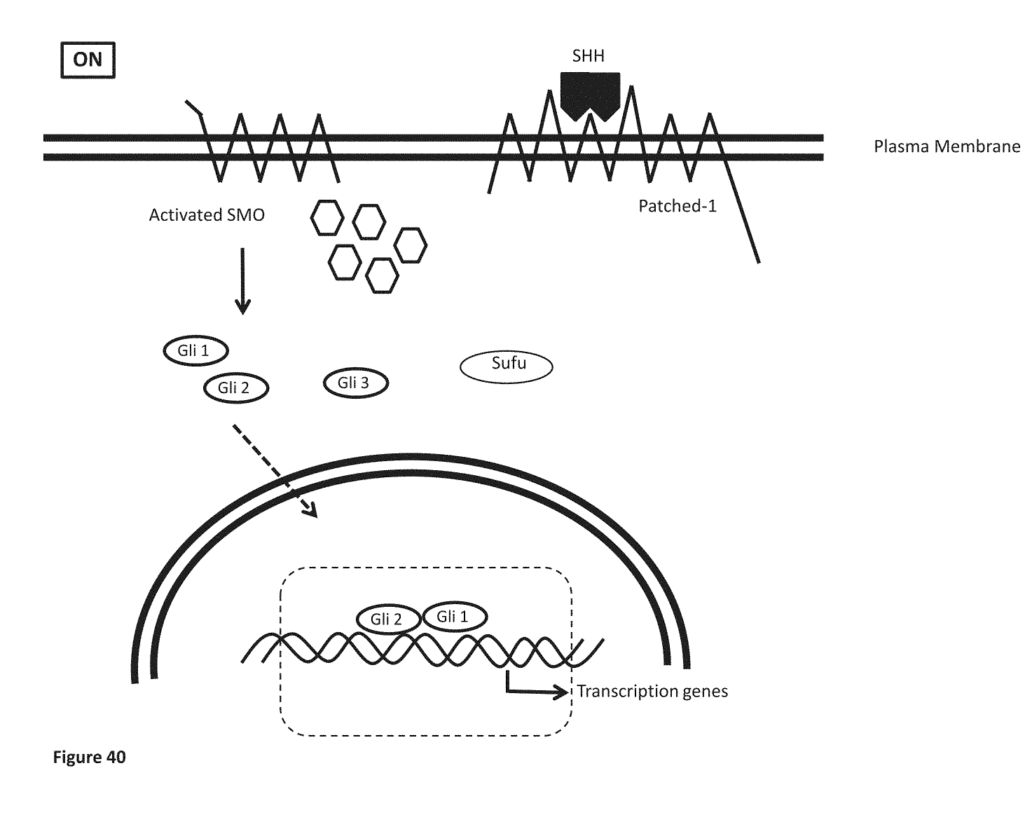

while the following table describes the “on” case.

| | | Type(s) of | Classes of | | Associated |
|---|---|---|---|---|---|
| Step | Description | Event | events | Constituents | FIGS. |
| 1 | On-Sonic Hedgehog | Degradation | Stoichiometric | SHH, | FIG. 36 |
| | (SHH) binds to Patched- | | | Patched-1 | |
| | 1, causing | | | | |
| | internalization and | | | | |
| | degradation. | | | | |
| 2 | The degradation of | Degradation | Membrane | Patched-1, SMO | FIG. 37 |
| | Patched-1 releases | | Transduction | | |
| | SMO, allowing it to | | | | |
| | enter the cilia. | | | | |
| 3 | When inside the cilia, | Dissociation | Stoichiometric | SMO, SUFU, | FIG. 38 |
| | SMO promotes the | | | Gli1, 2 | |
| | dissociation of a | | | | |
| | Suppressor of fused | | | | |
| | (SUFU)-glioma- | | | | |
| | associated oncogene | | | | |
| | homologue (Gli) | | | | |
| | complex. | | | | |
| 4 | Gli1, 2 enters the | Activation | Intracellular | Gli1, 2, 3 | FIG. 39 |
| | nucleus, resulting in | Degradation | Transport | | |
| | nuclear translocation, | | | | |
| | and activation of Gli1, & | | | | |
| | Gli2 transcription | | | | |
| | factors, & the | | | | |
| | degradation of the | | | | |
| | repressor Gli3. | | | | |
| 5 | Activated Gli | Transcription | Stoichiometric | Gli1, 2 | FIG. 40 |
| | transcription factors | | Gene | | |
| | accumulate in the | | Expression | | |
| | nucleus and controls | | | | |
| | gene transcription. | | | | |

The 5th column of the above table lists constituents of the pathway step described in that row of the table. These constituents would be used in a replica reaction executed by the present invention corresponding to the pathway step described in the associated row of the above table.

The named constituents involved the example contemporary understanding of the Hedgehog pathway represented by the above table can be produced in a laboratory or in some cases are commercially available or can be produced commercially in the future for use in the present invention and extensions of the present invention. As an example, the table below lists example sources for the constituents identified in the table above.

| Constituents | Description |
|---|---|
| SHH | The Sonic Hedgehog protein which initiates the signaling pathway by |
| | degradation of Patched-1 can be obtained from a supplier through this source: |
| | http://www.prospecbio.com/Sonic_Hedgehog_Human_3_186/(Visited on |
| | Sep. 8, 2014) |
| Patched-1 | The Patched-1 protein mentioned above, which inhibits SMO in the off state |
| | can be obtained from a supplier through this source: |
| | http://www.novusbio.com/Patched-1-Recombinant-Protein_H00005727-P01.html |
| | (Visited on Sep. 8, 2014) |
| SMO | The protein SMO can be obtained from a supplier through this source: |
| | http://www.abnova.com/products/products_detail.asp?catalog_id=H00006608-Q03 |
| | (Visited on Sep. 8, 2014) |
| SUFU | Suppressor of Fused (SUFU) can be obtained from a supplier through this |
| | source: |
| | http://www.abnova.com/products/products_detail.asp?catalog_id=H00051684-P01 |
| | (Visited on Sep. 8, 2014) |

### Example 3: The RTK Pathway

The MAPK signaling pathway regulate many cellular activities, including proliferation, differentiation, survival, and death. MAPK signaling pathways have also been implemented in developing many human diseases including Alzheimer's disease, Parkinson's disease, and amyotrophic lateral schlerosis.

The ERK signaling pathway contributes in several tumor developments. The phosphorylation by ERK of proteins, including myosin light chain kinase, focal adhesion kinase, and paxillin induces cancer cell migration. In addition, the ERK pathway also promotes activation of matrix metalloproteinases, which in turn promotes degradation of extracellular matrix proteins followed up with tumor invasion. ERK signaling regulates activities and levels of the proapoptotic protein BIM, and anti-apoptotic protein, MCL-1, which in turn promotes the survival of cancer cells. Increased MCL-1 is also associated with poor prognosis and resistance to anticancer drugs.

Mutations taken place in the EGFR, the activator of the ERK pathway, occur in the lung and colorectal cancers.

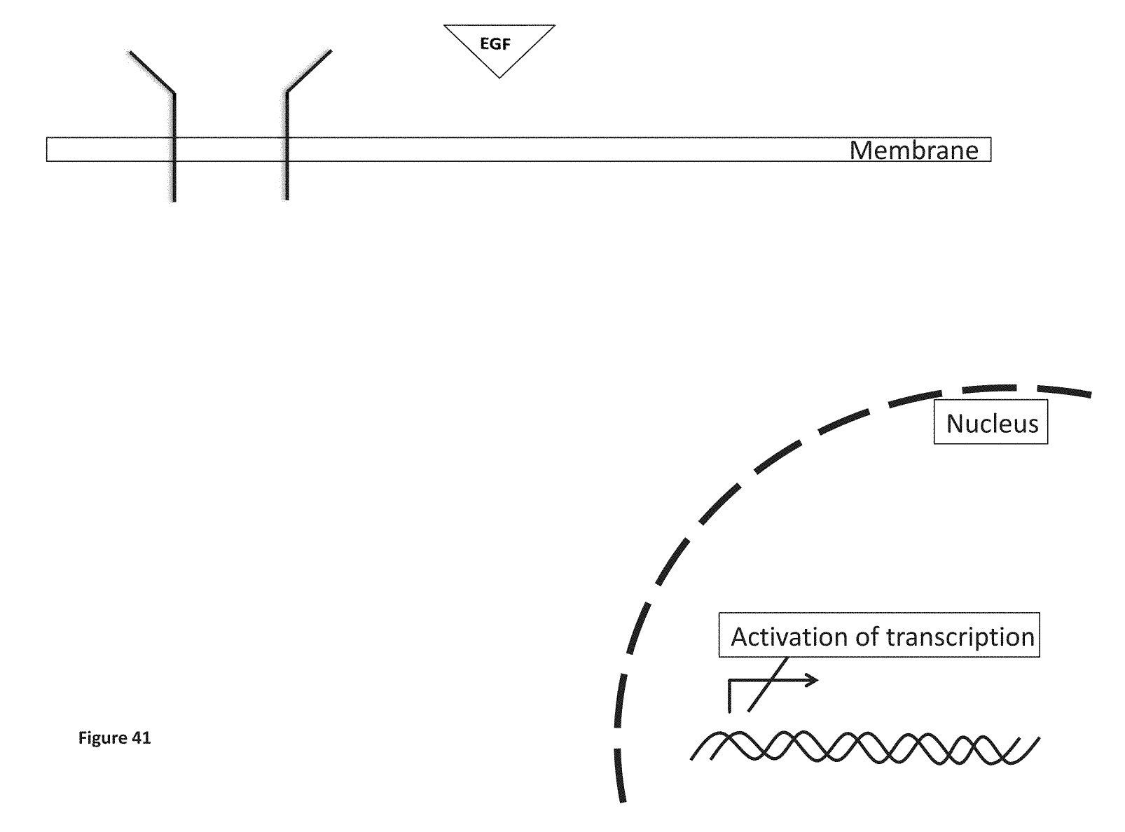

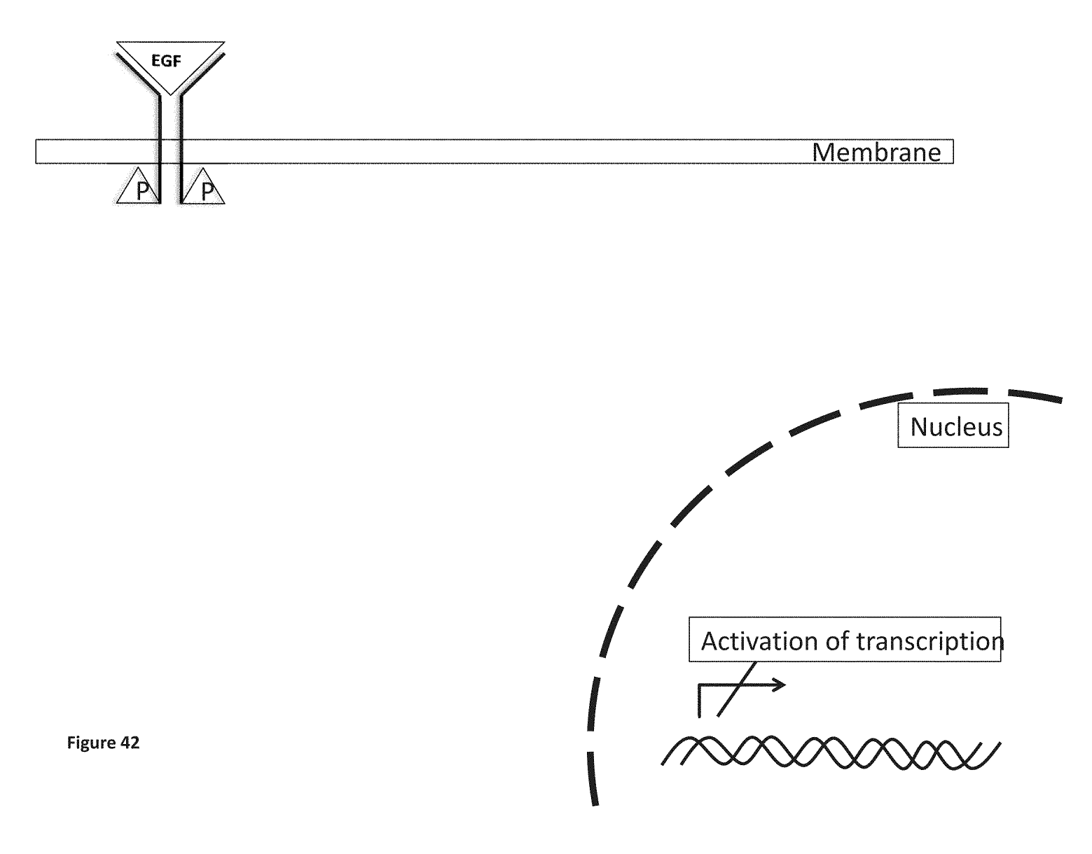

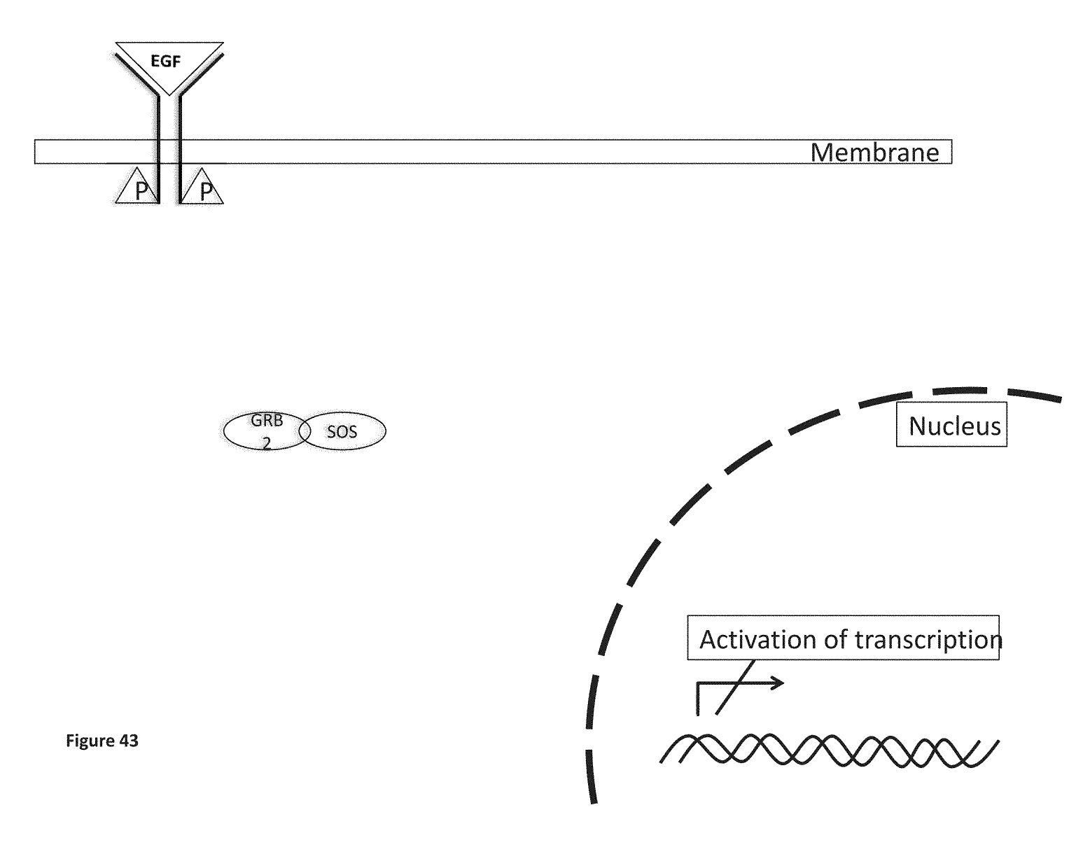

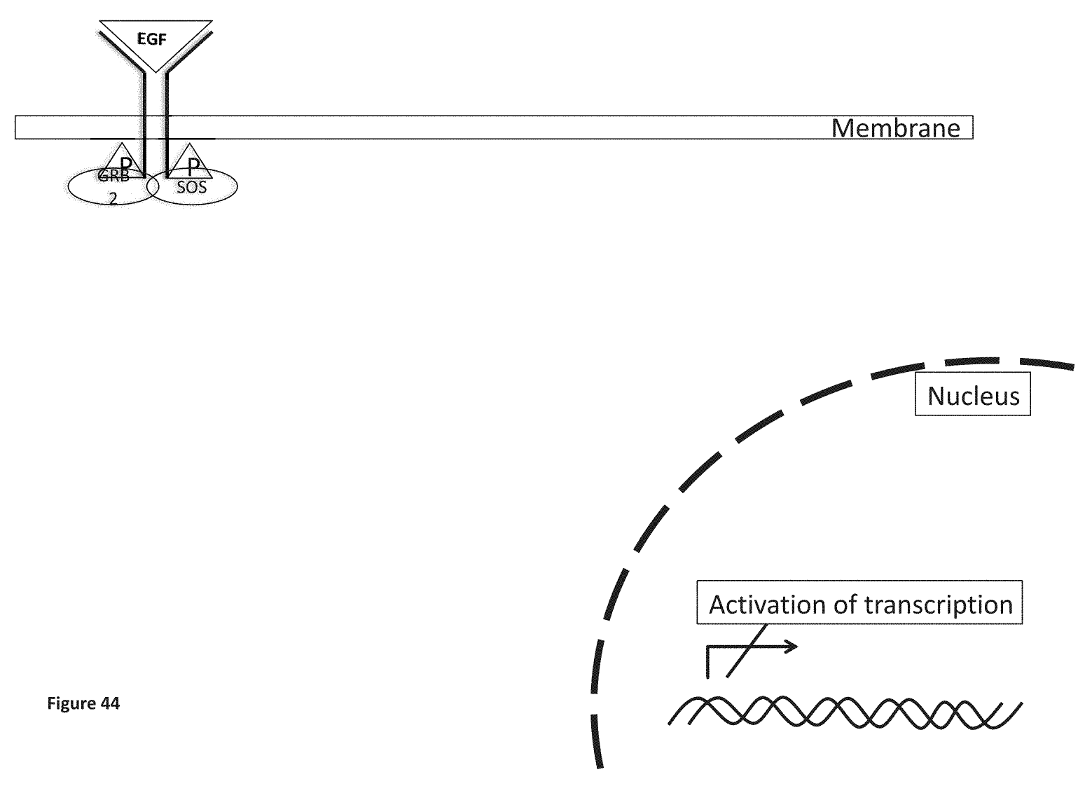

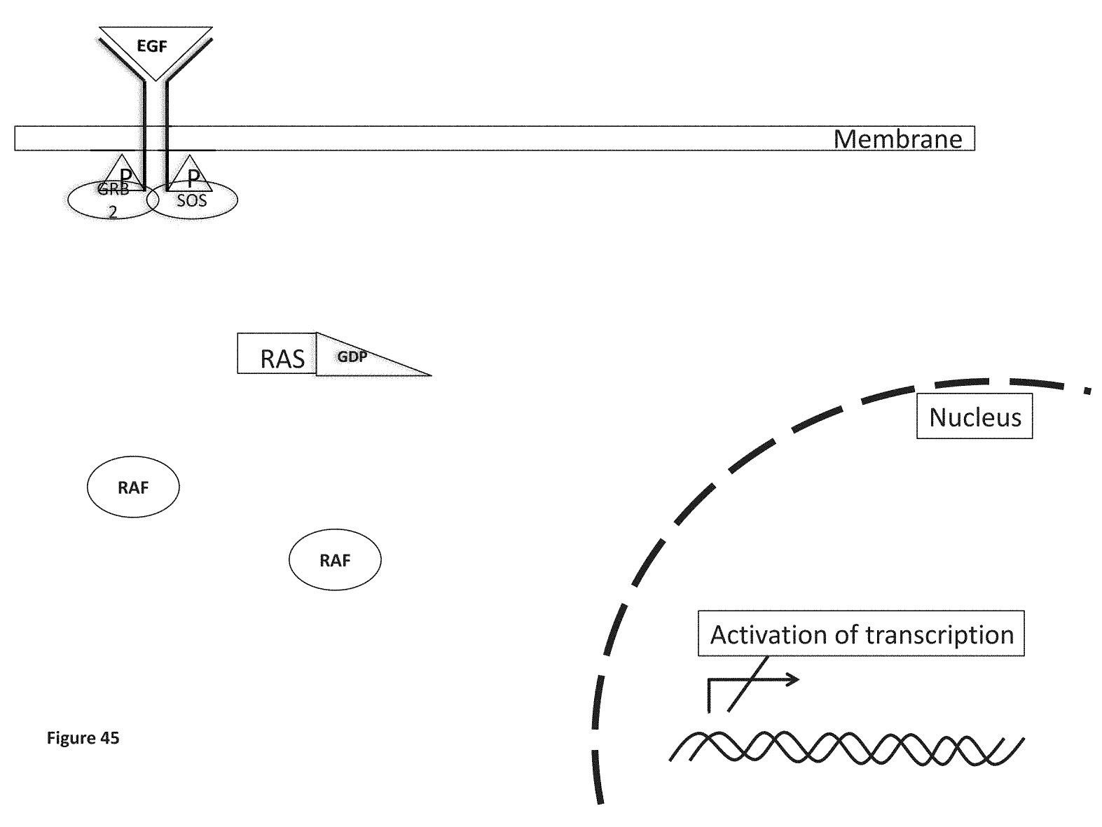

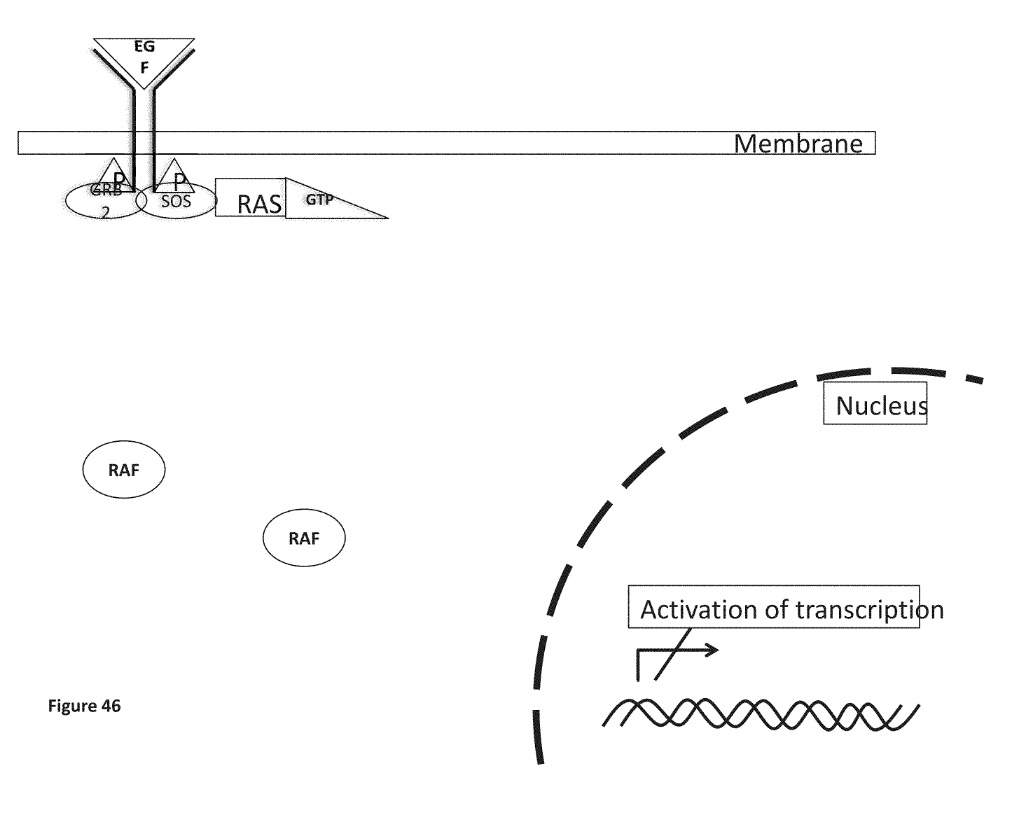

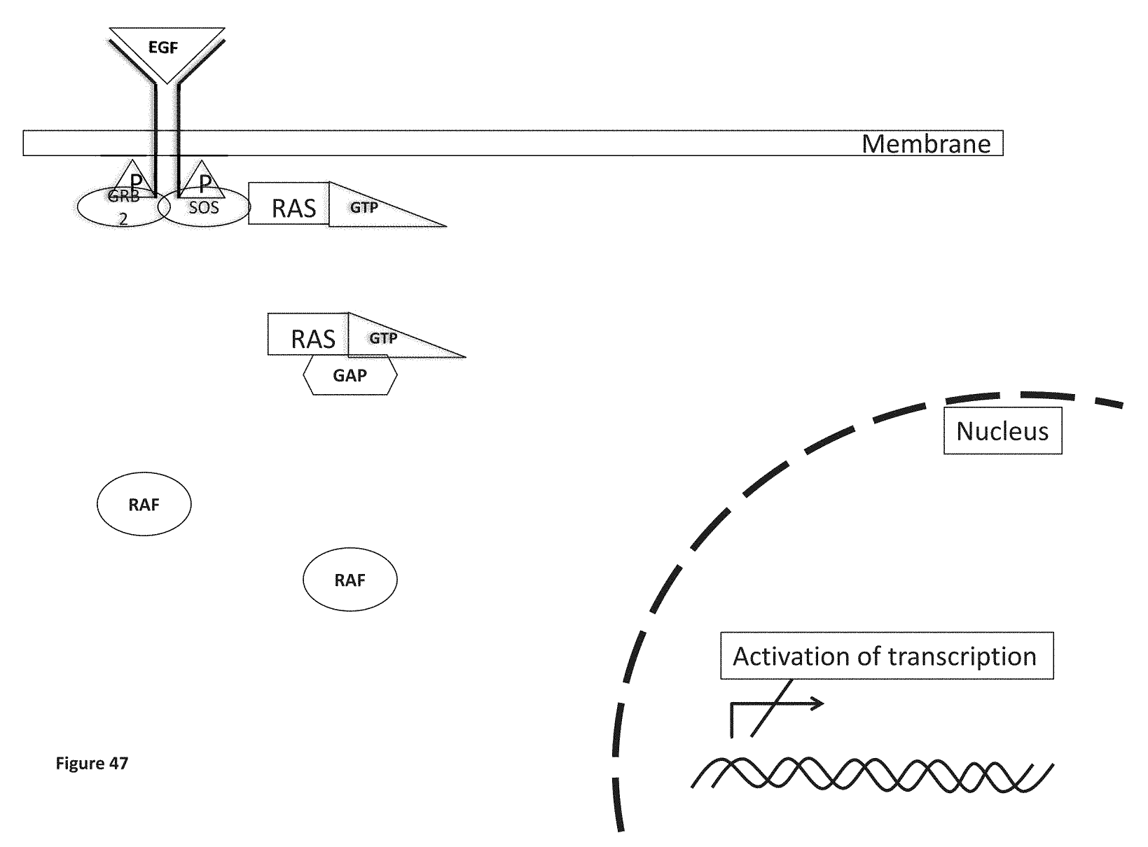

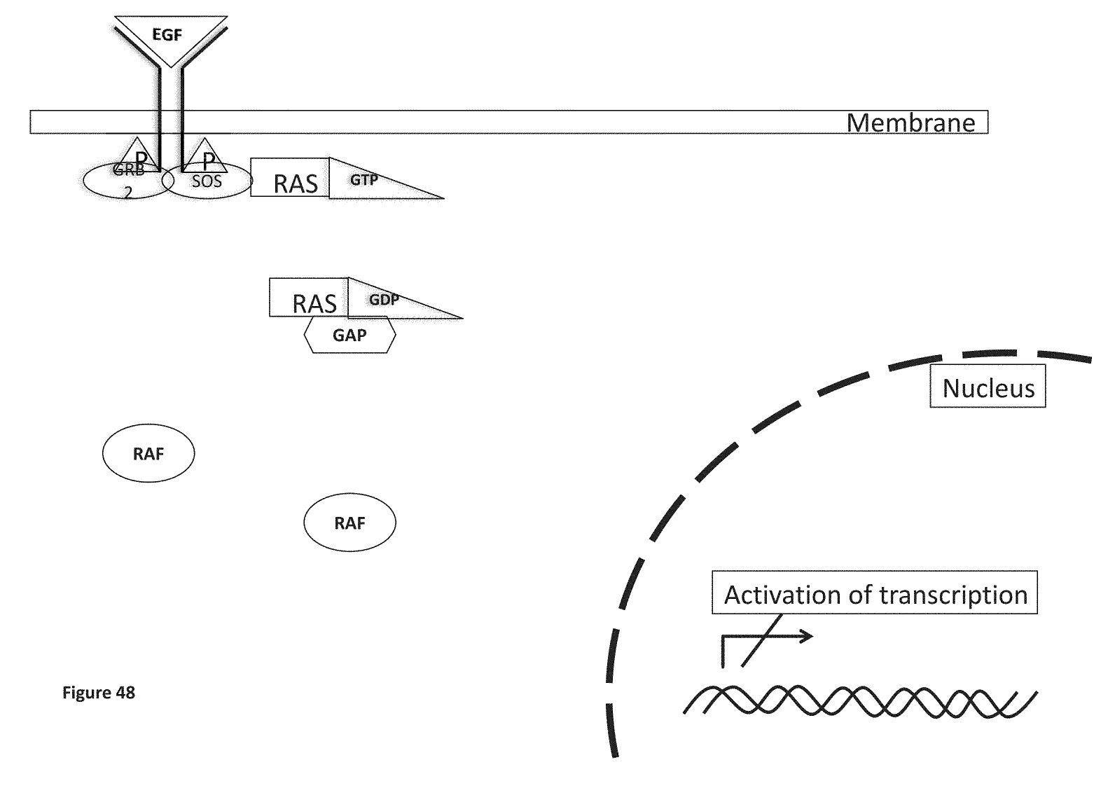

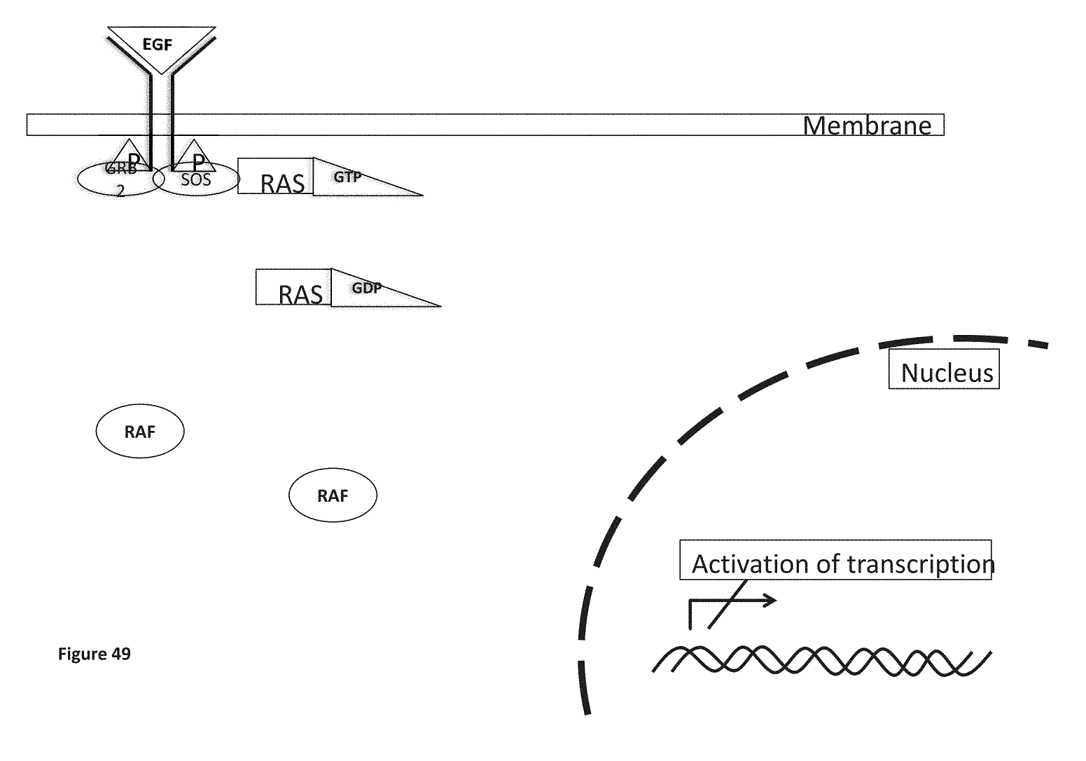

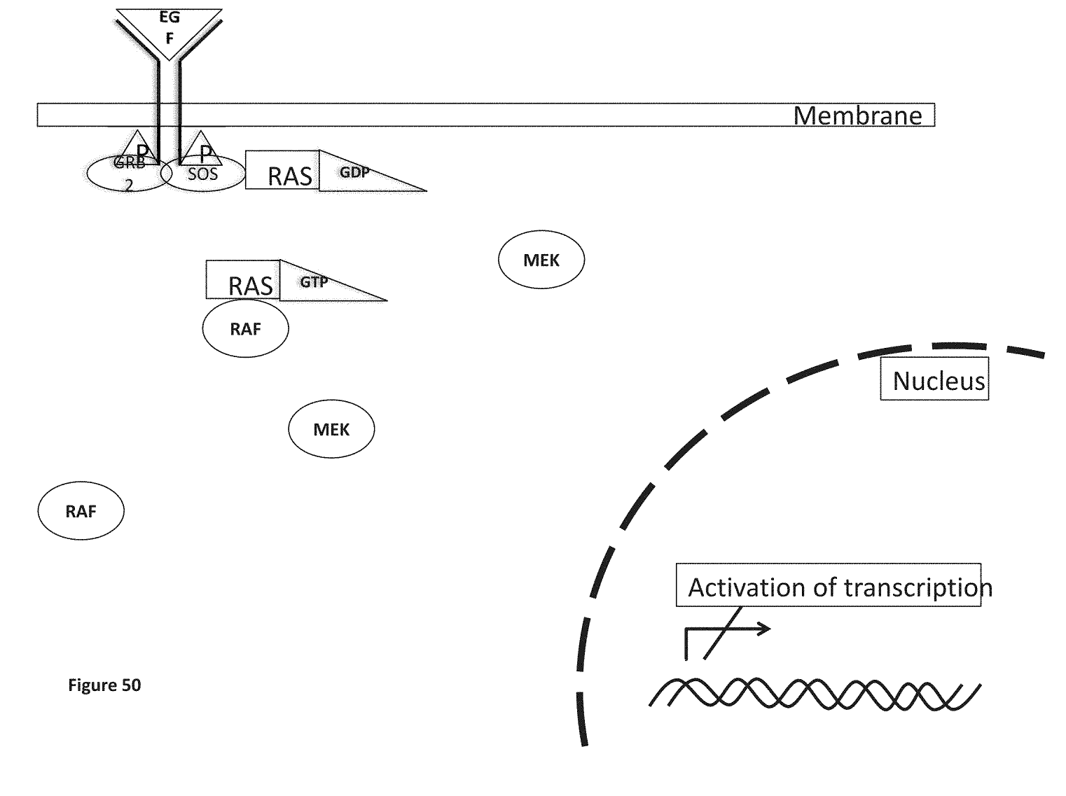

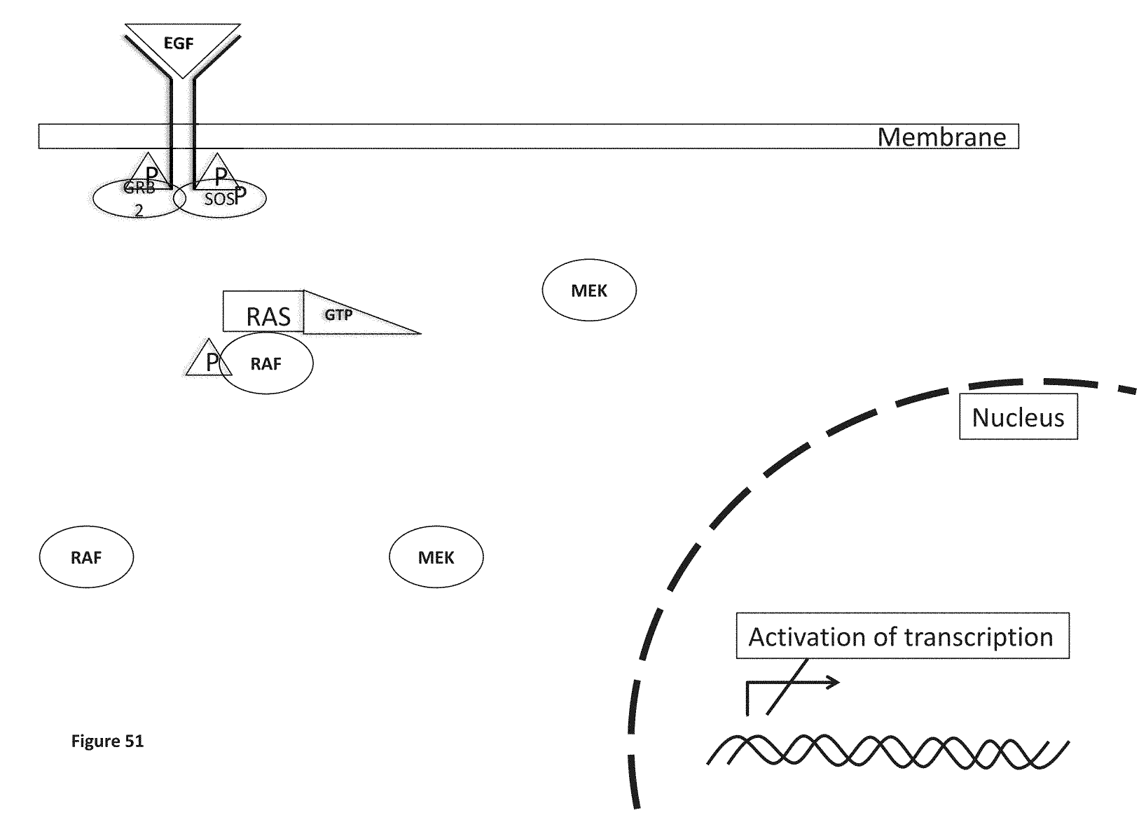

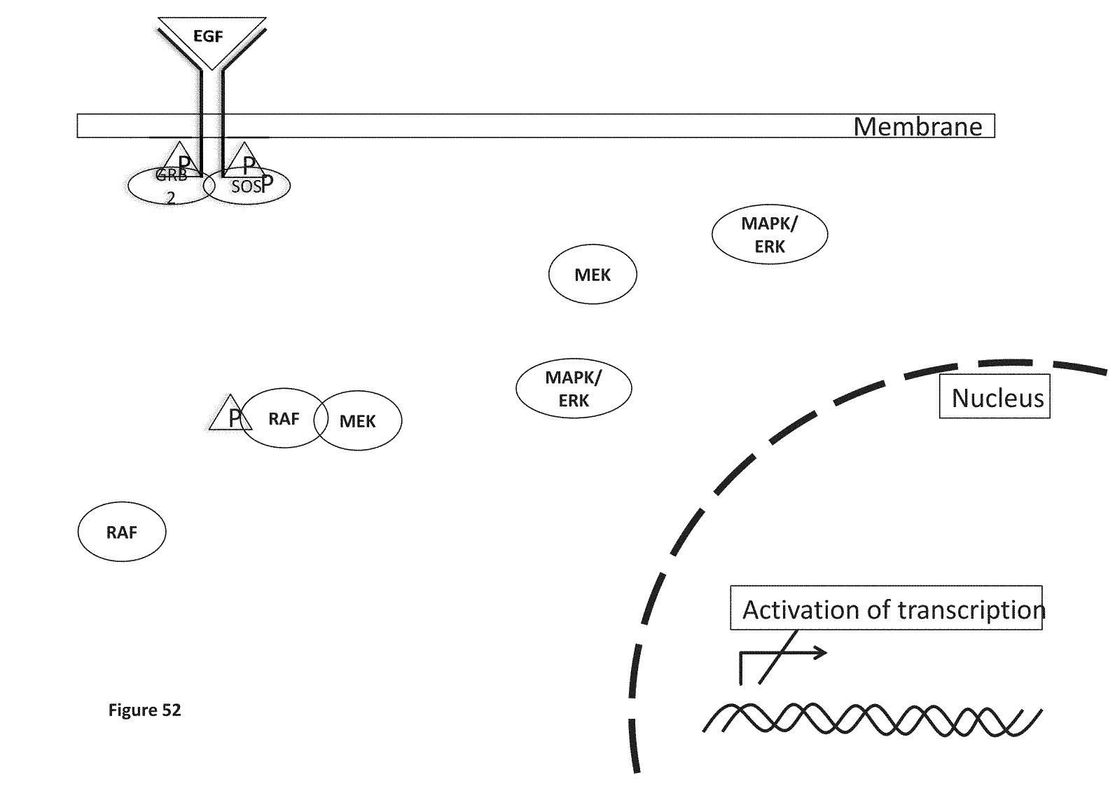

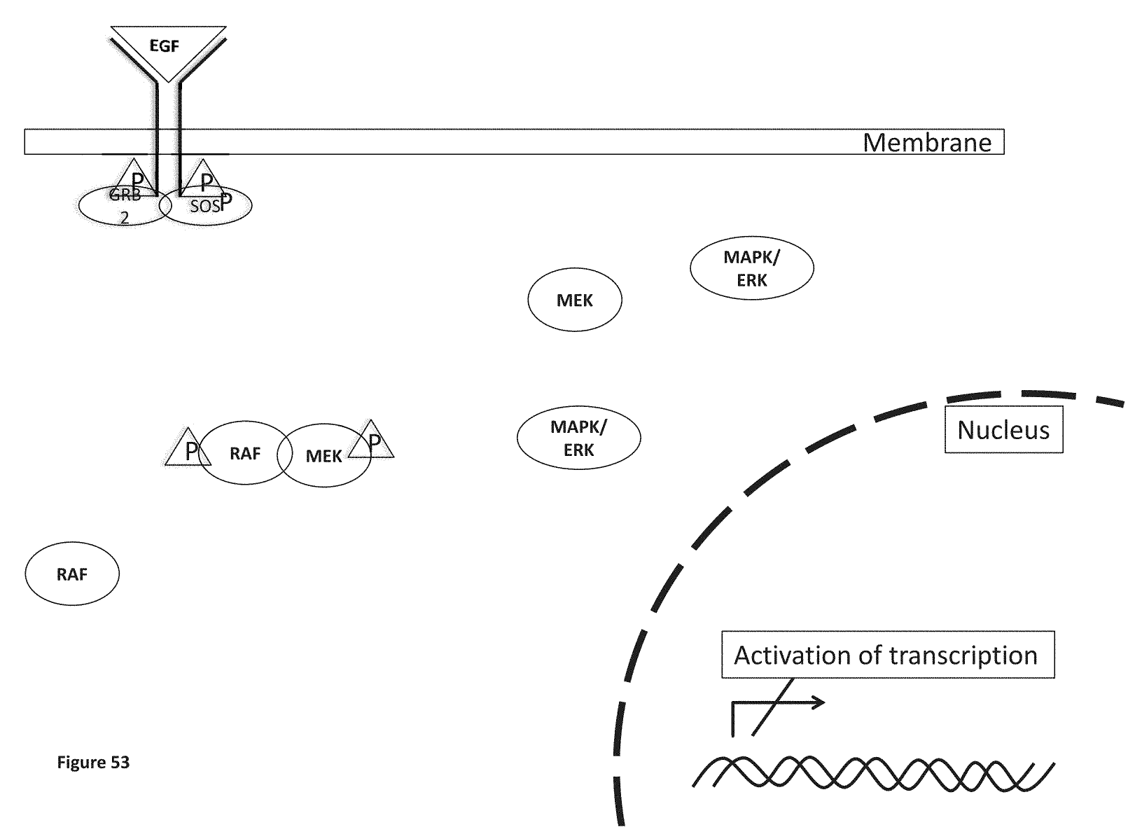

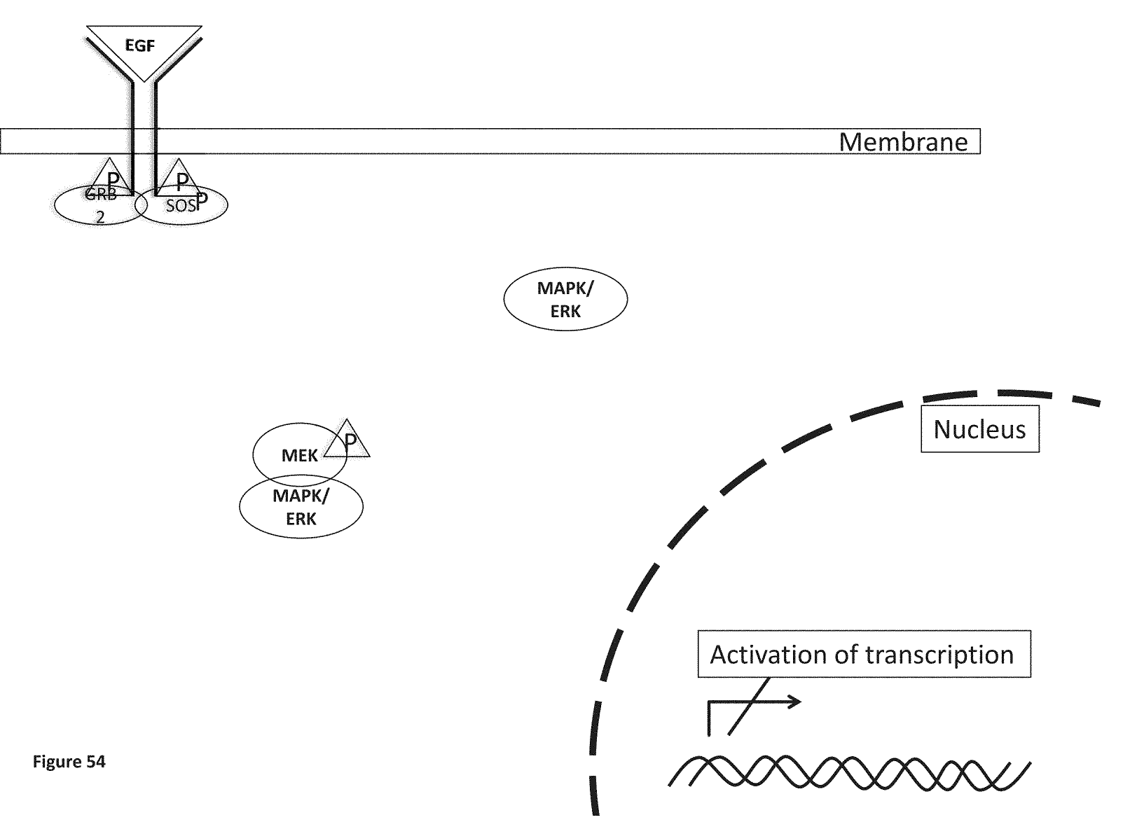

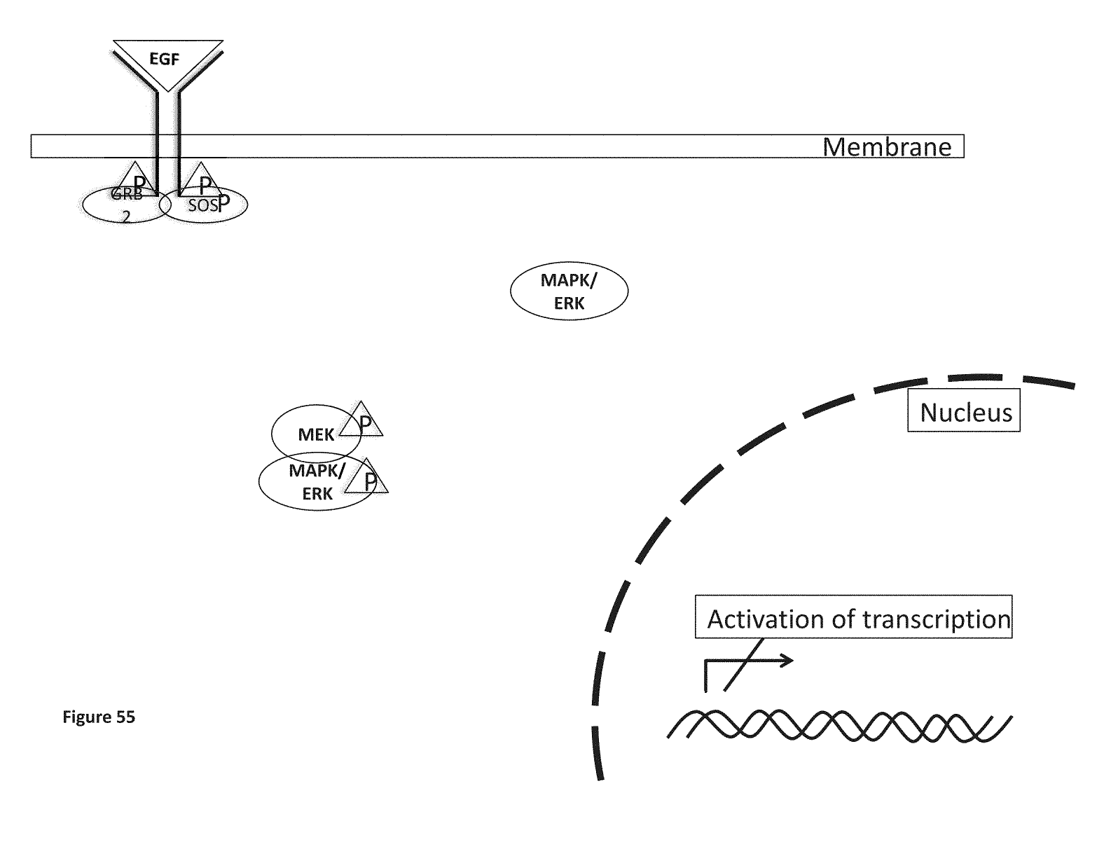

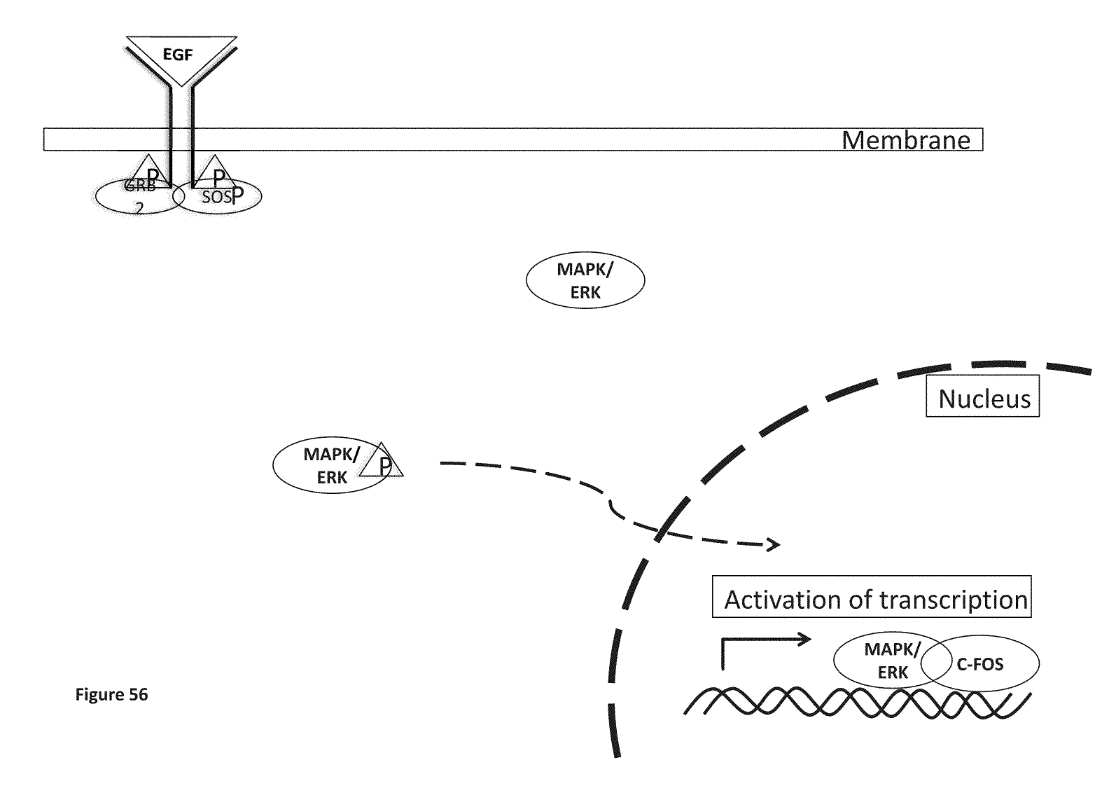

FIG. 41 through FIG. 56 and the following table provide example sequential step-by-step breakdown of an example contemporary understanding of the RTK pathway. Each step, represented by a table row and/or one of these figures, represents an example reaction step and is an example candidate for a replica reaction in the context of present invention and extensions of the present invention. As described earlier, the replica reactions can be implemented by embodiments of the present invention or extensions of the present invention in isolation (“stepwise”), and groups of contiguous steps can be implemented by embodiments of the present invention or extensions of the present invention together in a block of “one-pot” reactions (“blockwise”), the block representing a multiple-step segment or uninterrupted subset of the pathway. Example “stoichiometric” steps suitable for execution in the present invention are called out in the 4th column of the table. The 6th column of the table identifies example correspondences between the example contemporary understanding of the RTK pathway represented by the figures and example steps of the represented in of the table.

| | | Type(s) of | Classes of | | Associated |
|---|---|---|---|---|---|
| Step | Description | Event | events | Constituents | FIGS. |
| 1 | Epidermal growth | Binding | Membrane | EGF, EGFR | FIG. 41 |
| | factor (EGF) binds | Activation | Transduction | | |
| | to the receptor | | Stoichiometric | | |
| | EGFR to activate | | | | |
| | tyrosine kinase | | | | |
| | activity of | | | | |
| | cytoplasmic domain | | | | |
| | of the receptor. | | | | |
| 2 | EGFR becomes | Phosphorylation | Stoichiometric | EGFR | FIG. 42 |
| | phosphorylated. | | | | |
| 3 | The Docking | Binding | Stoichiometric | GRB2, SOS | FIG. 43 |
| | protein, GRB2 binds | | | | |
| | to SOS. | | | | |
| 4 | When GRB2-SOS | Activation | Stoichiometric | GRB2-SOS complex, | FIG. 44 |
| | complex docks to | | | EGFR | |
| | phosphorylated | | | | |
| | EGFR, SOS | | | | |
| | becomes activated. | | | | |
| 5 | SOS catalyzes the | Removal | Stoichiometric | SOS, GDP, Ras, GTP | FIG. 45 |
| | exchange of GDP for | Binding | | | FIG. 46 |
| | GTP. | Activation | | | |
| | (The activated SOS | | | | |
| | removes GDP from | | | | |
| | Ras, which in turn | | | | |
| | allows Ras to bind | | | | |
| | GTP to become | | | | |
| | active.) | | | | |
| 6 | GAP promotes | Hydrolysis | Stoichiometric | GAP, GTP, GDP | FIG. 47, |
| | hydrolysis of GTP to | | | | FIG. 48, |
| | GDP. | | | | FIG. 49 |
| 7 | Activated Ras | Phosphorylation | Stoichiometric | Ras, RAF kinase | FIG. 50 |
| | activates the protein | Activation | | | |
| | kinase activity of | | | | |
| | RAF kinase. | | | | |
| 8 | RAF kinase | Phosphorylation | Stoichiometric | RAF kinase, MEK | FIG. 51, |
| | phosphorylates and | Activation | | | FIG. 52 |
| | activates MEK. | | | | |
| 9 | MEK phosphorylates | Phosphorylation | Stoichiometric | MEK, MAPK | FIG. 53, |
| | and activates | Activation | | | FIG. 54, |
| | MAPK. | | | | FIG. 55 |
| 10 | MAPK activation | Translation | Intracellular | MAPK, mRNA | FIG. 56 |
| | alters the translation | | Transport | | |
| | of mRNA to | | | | |
| | proteins. | | | | |
| 11 | MAPK also | Transcription | Stoichiometric | MAPK, transcription | FIG. 56 |
| | regulates the | | Gene expression | factors, | |
| | activities of several | | | C-Fos | |
| | transcription factors, | | | | |
| | and regulates the | | | | |
| | transcription of the | | | | |
| | C-Fos gene. | | | | |

The 5th column of the above table lists constituents of the pathway step described in that row of the table. These constituents would be used in a replica reaction executed by the present invention corresponding to the pathway step described in the associated row of the above table.

The named constituents involved the example contemporary understanding of the RTK pathway represented by the above table can be produced in a laboratory or in some cases are commercially available or can be produced commercially in the future for use in the present invention and extensions of the present invention. As an example, the table below lists example sources for the constituents identified in the table above.

| Constituents | Description |
|---|---|
| EGF | The EGF protein, which binds with the EGFR ligands to start the signaling |
| | pathway can be obtained from a supplier through this source: |
| | http://www.lifetechnologies.com/order/catalog/product/PHG0311 |
| | (Visited on Sep. 8, 2014) |
| EGFR | http://www.lifetechnologies.com/order/catalog/product/PV4803 |
| | (Visited on Sep. 8, 2014) |
| GRB2 | http://www.abnova.com/products/products_detail.asp?catalog_id=H00002885-Q01 |
| | (Visited on Sep. 8, 2014) |
| SOS | http://www.enzolifesciences.com/BML-P305/sos-1-human-1149-1158/ |
| | http://www.abnova.com/products/products_detail.asp?catalog_id=H00006654-Q01 |
| | (Visited on Sep. 8, 2014) |
| Ras | http://www.cellbiolabs.com/ras-recombinant-proteins Ras |
| | http://www.sinobiological.com/K-Ras-Protein-g-4979.html Ras |
| | (Visited on Sep. 8, 2014) |
| RAF kinase | http://www.sigmaaldrich.com/catalog/product/sigma/r3652?lang=en&region=US |
| | (Visited on Sep. 8, 2014) |
| MEK | http://www.abdserotec.com/human-mek-1-antibody-5812- |
| | hca067.html?f=purified (antibody) |
| | http://www.enzolifesciences.com/BML-SE509/mek1-human-recombinant-his-tag/MEK1 |
| | (Visited on Sep. 8, 2014) |
| MAPK | https://www.caymanchem.com/app/template/Product.vm/catalog/10009179 (antibody) |
| | http://www.biocompare.com/pfu/110627/soids/2-143578/Assay_Kit/ELIS A_Mitogen_Activated_Protein_Kinase_Kinase_1 |
| | (Visited on Sep. 9, 2014) |
| C-Fos | http://www.novusbio.com/c-Fos-Recombinant-Protein_H00002353-P01.html |
| | (Visited on Sep. 10, 2014) |

### Example 4: The JAK-STAT Pathway

The JAK-STAT signaling pathway mediates cellular responses to many of cytokines and growth factors, such as proliferation, differentiation, migration, and apoptosis. All of these responses are crucial for the development and homeostasis of hematopoietic cells. In mammalian systems, JAK/STAT signaling plays a key role in controlling organ or tissue size.

Disruption or mutation of the homeostatic process of JAK-STAT activation may lead to oncogenic results. Genetic abnormalities that induce active JAK-STAT signaling have been demonstrated in many hematologic malignancies. In addition, somatic JAK2 mutations have been identified in many patients with myeloproliferative neoplasm.

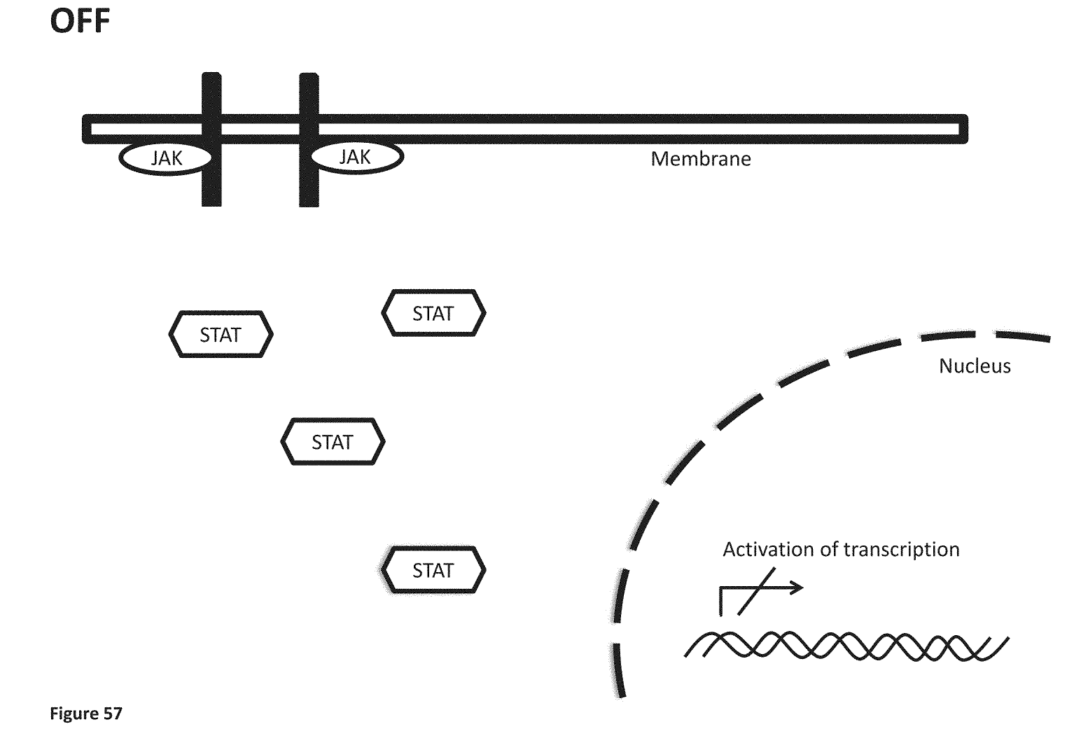

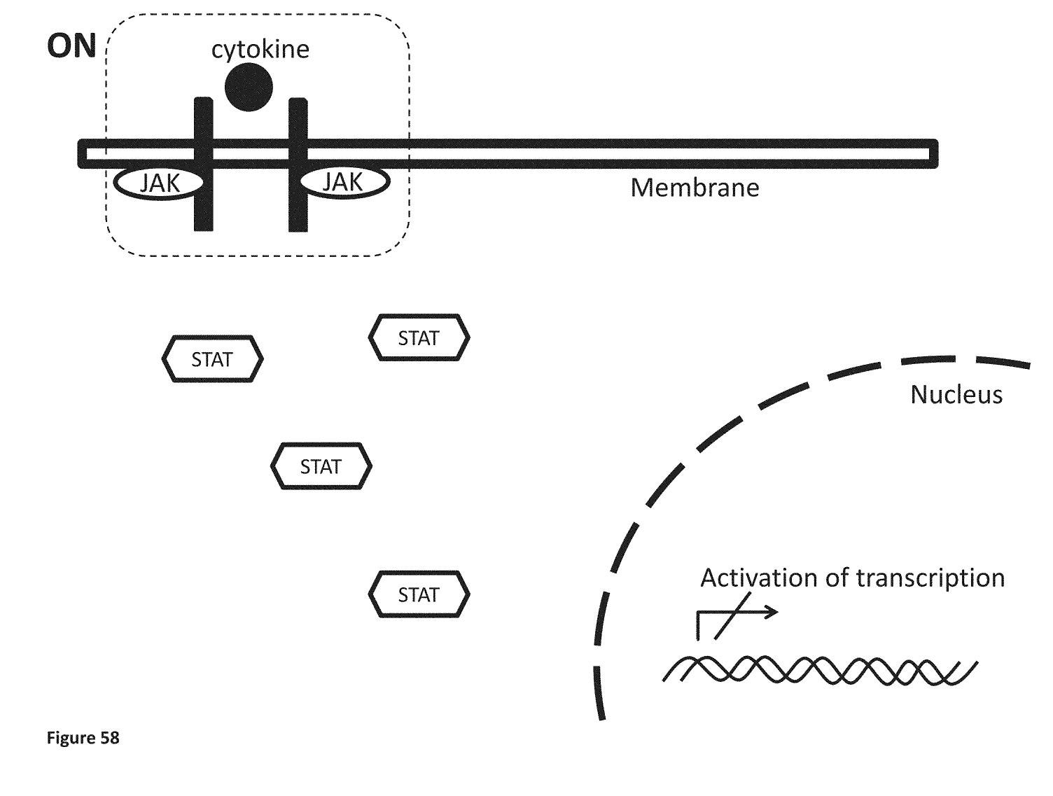

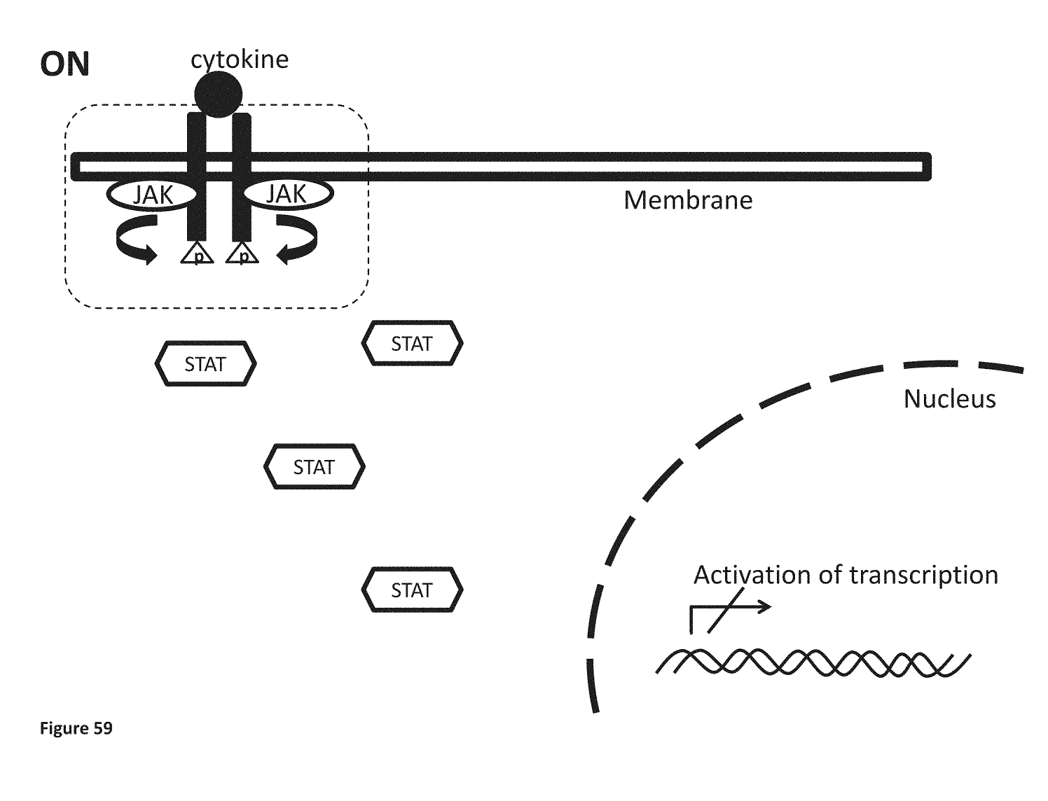

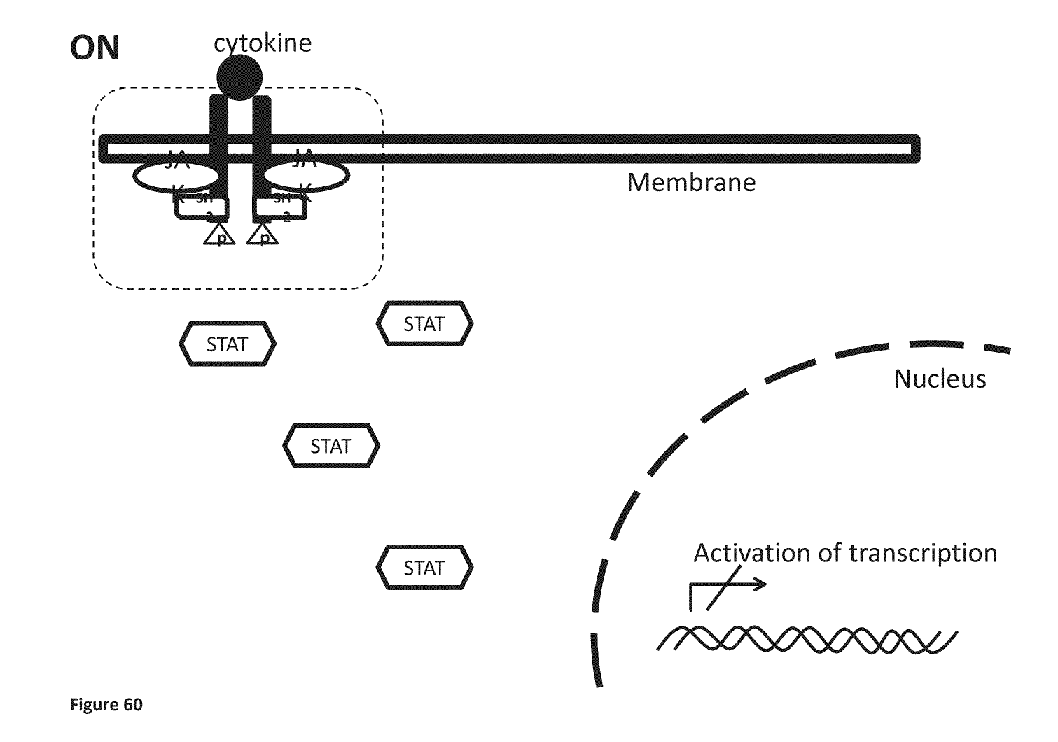

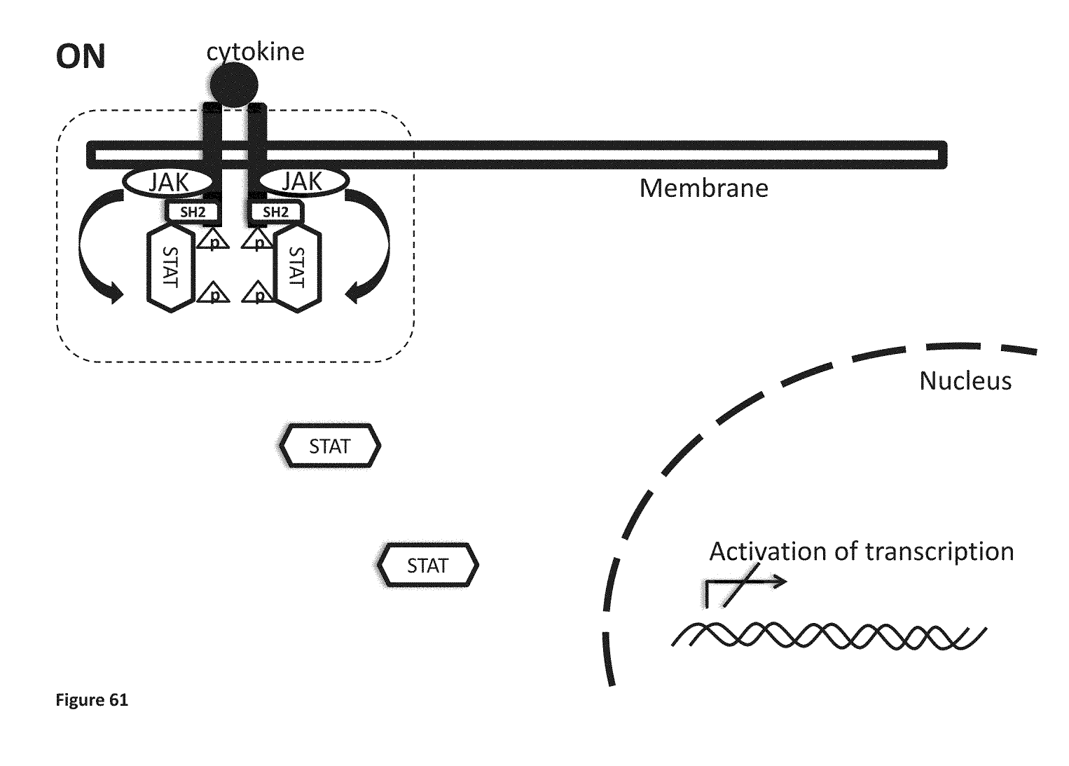

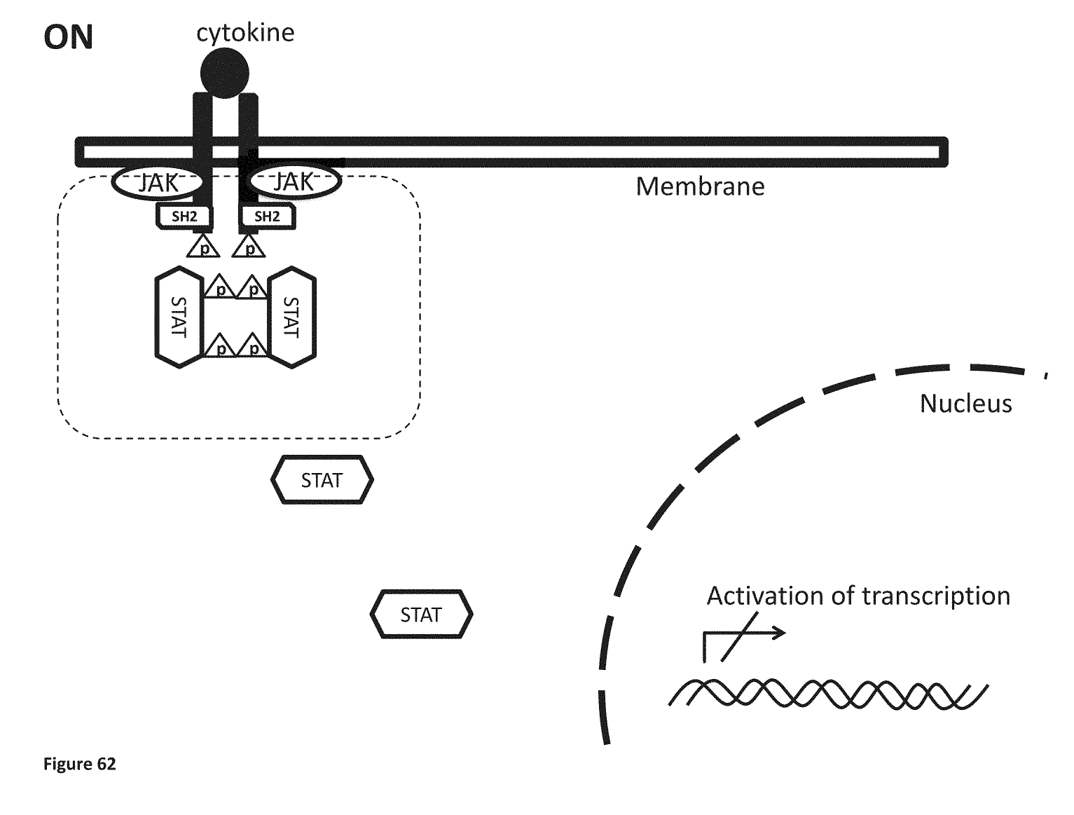

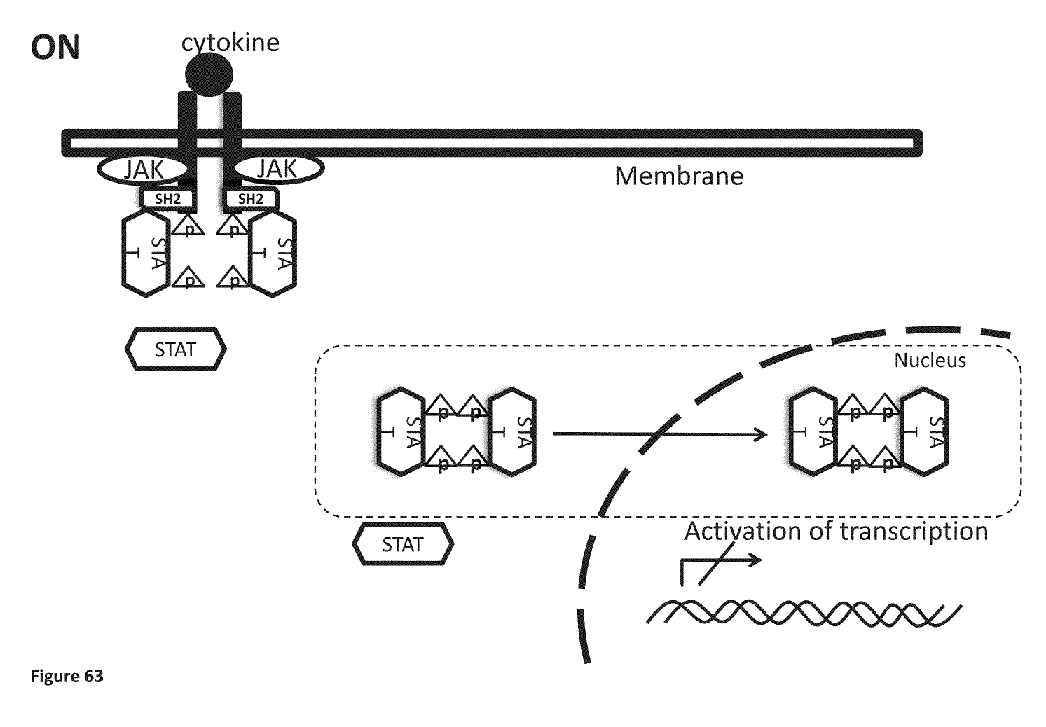

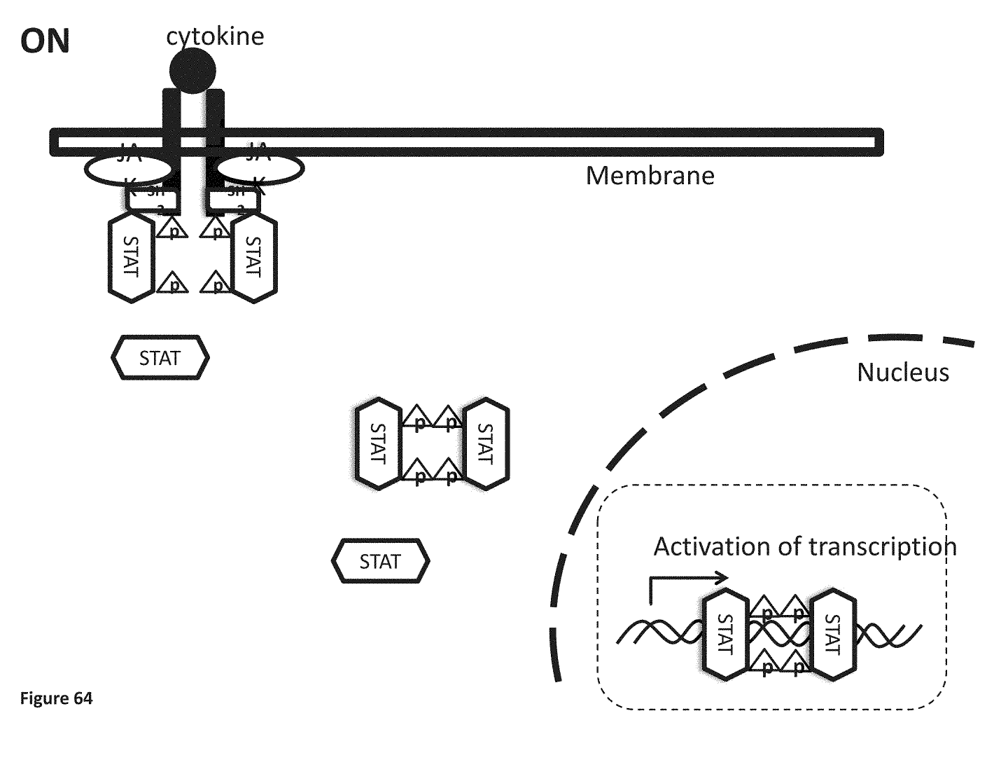

FIG. 57 through FIG. 64 and the following table provide example sequential step-by-step breakdown of an example contemporary understanding of the JAK-STAT pathway. Each step, represented by a table row and/or one of these figures, represents an example reaction step and is an example candidate for a replica reaction in the context of present invention and extensions of the present invention. As described earlier, the replica reactions can be implemented by embodiments of the present invention or extensions of the present invention in isolation (“stepwise”), and groups of contiguous steps can be implemented by embodiments of the present invention or extensions of the present invention together in a block of “one-pot” reactions (“blockwise”), the block representing a multiple-step segment or uninterrupted subset of the pathway. Example “stoichiometric” steps suitable for execution in the present invention are called out in the 4th column of the table. The 6th column of the table identifies example correspondences between the example contemporary understanding of the JAK-STAT pathway represented by the figures and the example steps of represented in of the table.

| | | Type of | Classes of | | Associated |
|---|---|---|---|---|---|
| Step | Description | Event | events | Constituents | FIGS. |
| 1 | JAKs binds to the | Binding | Membrane | JAKs, cytokine | FIG. 57 |
| | cell surface of | Activation | Transduction | receptors | |
| | cytokine | | Stoichiometric | | |
| | receptors. | | | | |
| 2 | Binding of the | Activation | Stoichiometric | Ligands, JAKs | FIG. 58 |
| | ligands (peptides | Dimerization | | | |
| | eg. cytokines) to | | | | |
| | the receptor | | | | |
| | activates JAKs. | | | | |
| 3 | JAKs | Phosphorylation | Stoichiometric | JAKs, tyrosine | FIG. 59 |
| | phosphorylates | | | residues, SH2 | |
| | tyrosine residues | | | domains | |
| | on the receptor. | | | | |
| 4 | Phosphorylated | Site Creation | Stoichiometric | p-Tyrosine | FIG. 60 |
| | tyrosine residues | | | residues, proteins | |
| | create sites for | | | containing | |
| | interaction with | | | phosphotyrosine- | |
| | proteins | | | bind SH2 | |
| | containing | | | domains | |
| | phosphotyrosine- | | | | |
| | bind SH2 | | | | |
| | domains. | | | | |
| 5 | STATs that | Binding | Membrane | STATs, SH2 | FIG. 61 |
| | possess SH2 | | Transduction | domains capable | |
| | domains capable | | Stoichiometric | of JAKs | |
| | of binding the | | | | |
| | phosphotyrosine | | | | |
| | residues are | | | | |
| | recruited to the | | | | |
| | receptors. | | | | |
| 6 | STATs are then | Phosphorylation | Stoichiometric | STATs, JAKs | FIG. 62 |
| | phosphorylated by | | | | |
| | JAKs. | | | | |
| 7 | Two STATs | Dimerization | Stoichiometric | STATs | FIG. 63 |
| | forms STATs | Activation | | | |
| | dimers and are | | | | |
| | activated. | | | | |
| 8 | Activated STAT | Transcription | Intracellular | STAT, | FIG. 64 |
| | dimers | | Transport | target genes | |
| | accumulate in the | | Stoichiometric | | |
| | nucleus and | | Gene Expression | | |
| | activate | | | | |
| | transcription of | | | | |
| | target genes. | | | | |

The 5th column of the above table lists constituents of the pathway step described in that row of the table. These constituents would be used in a replica reaction executed by the present invention corresponding to the pathway step described in the associated row of the above table.

The named constituents involved the example contemporary understanding of the JAK-STAT pathway represented by the above table can be produced in a laboratory or in some cases are commercially available or can be produced commercially in the future for use in the present invention and extensions of the present invention. As an example, the table below lists example sources for some of the constituents identified in the table above.

| Constituents | Description |
|---|---|
| JAKs | http://www.abnova.com/products/products_detail.asp?catalog_id=H00003716-Q01 |
| | JAK1(Visited on Sep. 10, 2014) |
| | http://www.abnova.com/products/products_detail.asp?catalog_id=H00003718-P01 |
| | JAK3 (Visited on Sep. 10, 2014) |
| STATs | http://www.abnova.com/products/products_detail.asp?catalog_id=H00006773-Q01 |
| | STAT2 (Visited on Sep. 10, 2014) |
| | http://www.abnova.com/products/products_detail.asp?catalog_id=H00006774-P01 |
| | STAT3 (Visited on Sep. 10, 2014) |
| | http://www.abnova.com/products/products_detail.asp?catalog_id=H00006775-P01 |
| | STAT4 (Visited on Sep. 10, 2014) |
| | http://www.abnova.com/products/products_detail.asp?catalog_id=H00006776-P01 |
| | STAT5 (Visited on Sep. 10, 2014) |
| | http://www.abnova.com/products/products_detail.asp?catalog_id=H00006778-P01 |
| | STAT6 (Visited on Sep. 10, 2014) |

### Example 5: The Notch Pathway

The Notch signaling pathway plays a crucial role in cell-cell communication, and regulation of embryonic development. Notch signaling is essential in the regulation of polarity and during left-right asymmetry determination. Mutation experiments have shown that, in the absence of Notch signaling, abnormal anterior-posterior polarity in somites occurs.

The Notch signaling is essential for sustaining NPCs in the developing brain. Loss-of-function mutations in the pathway cause precocious neuronal differentiation and NPC depletion. The NUMB protein is capable of antagonizing Notch effects, hindering the cell cycle and differentiation of NPCs. Notch signaling controls NPC self-renewal and cell fate specification.

Notch also plays an important role in cardiac development. Notch signaling regulates the atrioventricular boundary formation between the atrioventricular and the myocardium. Notch signaling is also involved in the ventricular endocardium, and is required for proper trabeculae development after myocardial specification. Notch signaling is also found to be involved in stages of pancreatic development, intestinal development, and bone development.

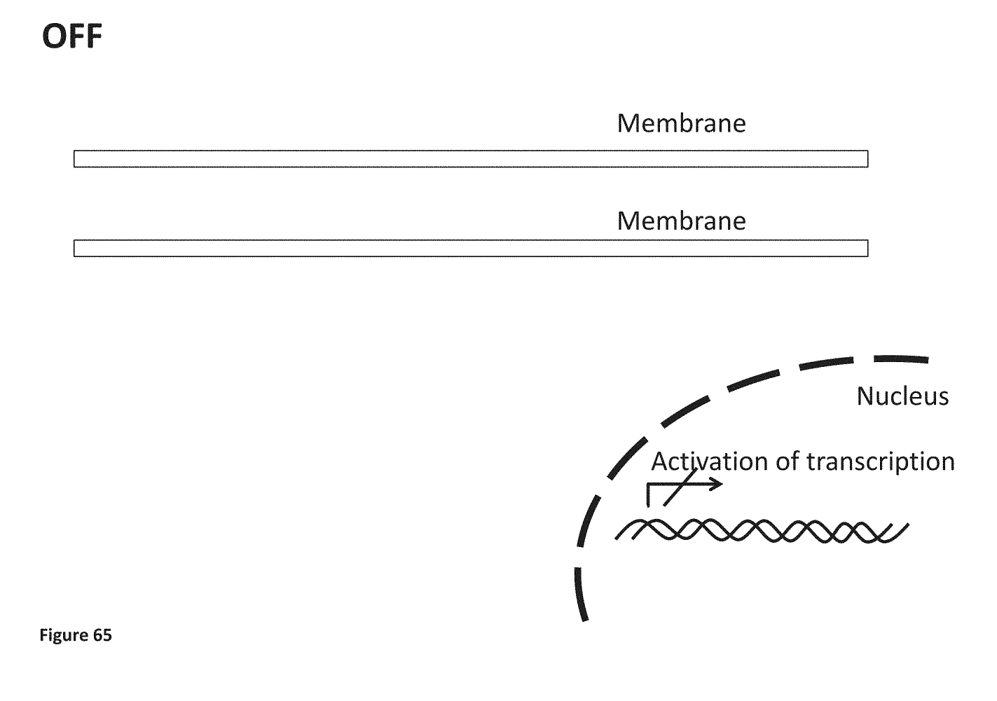

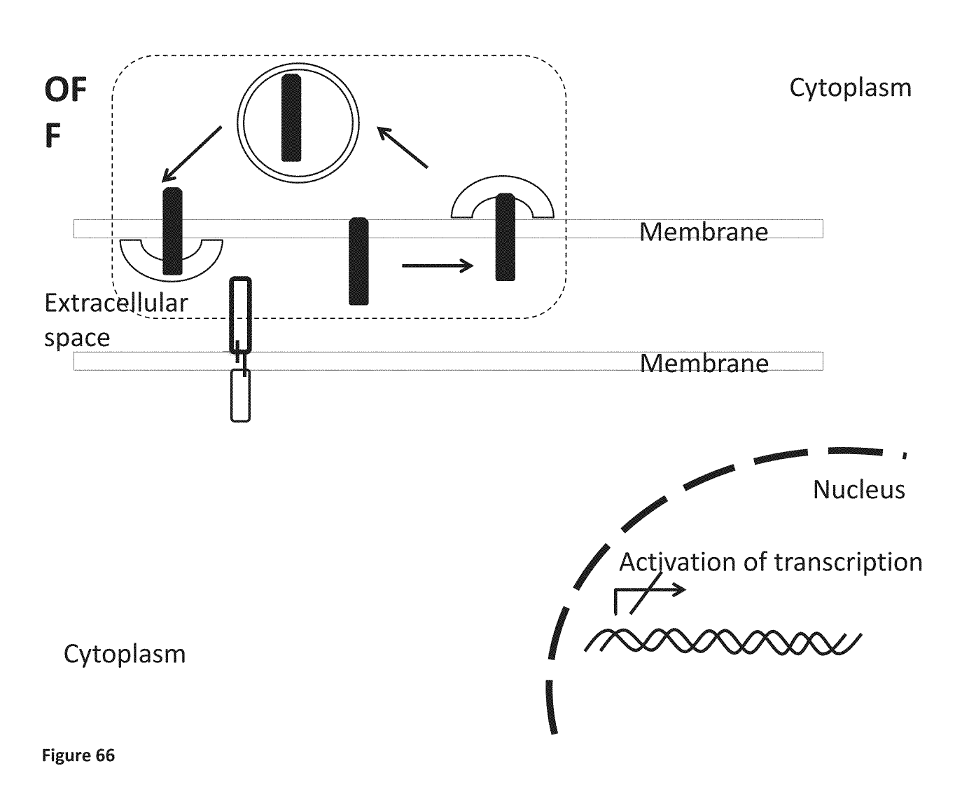

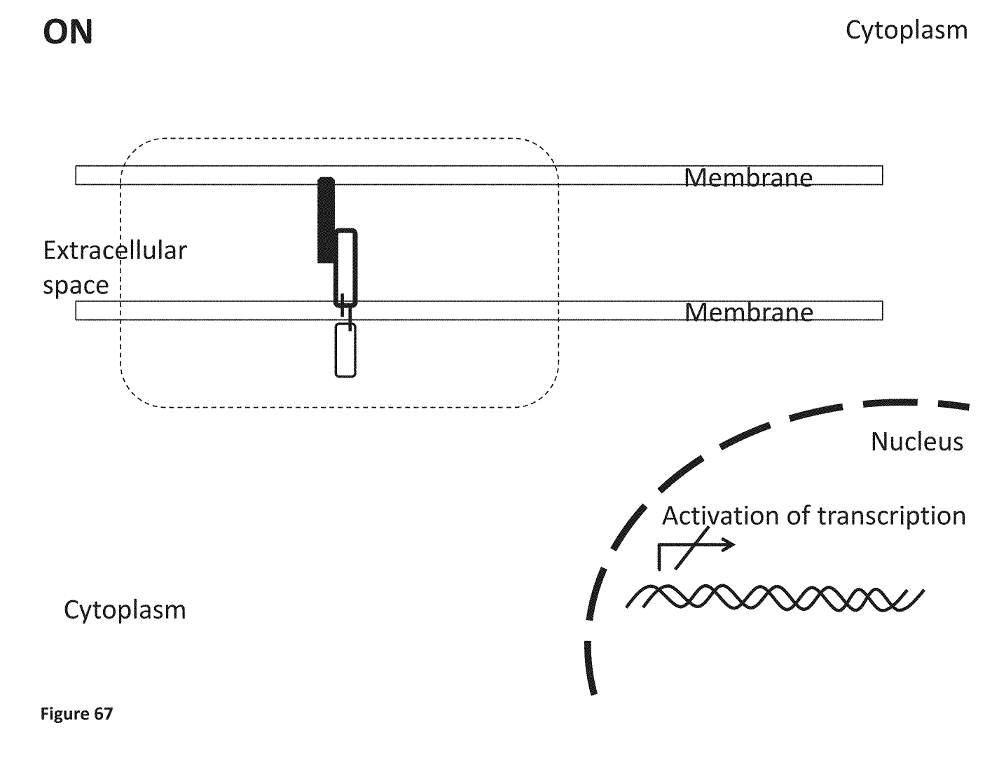

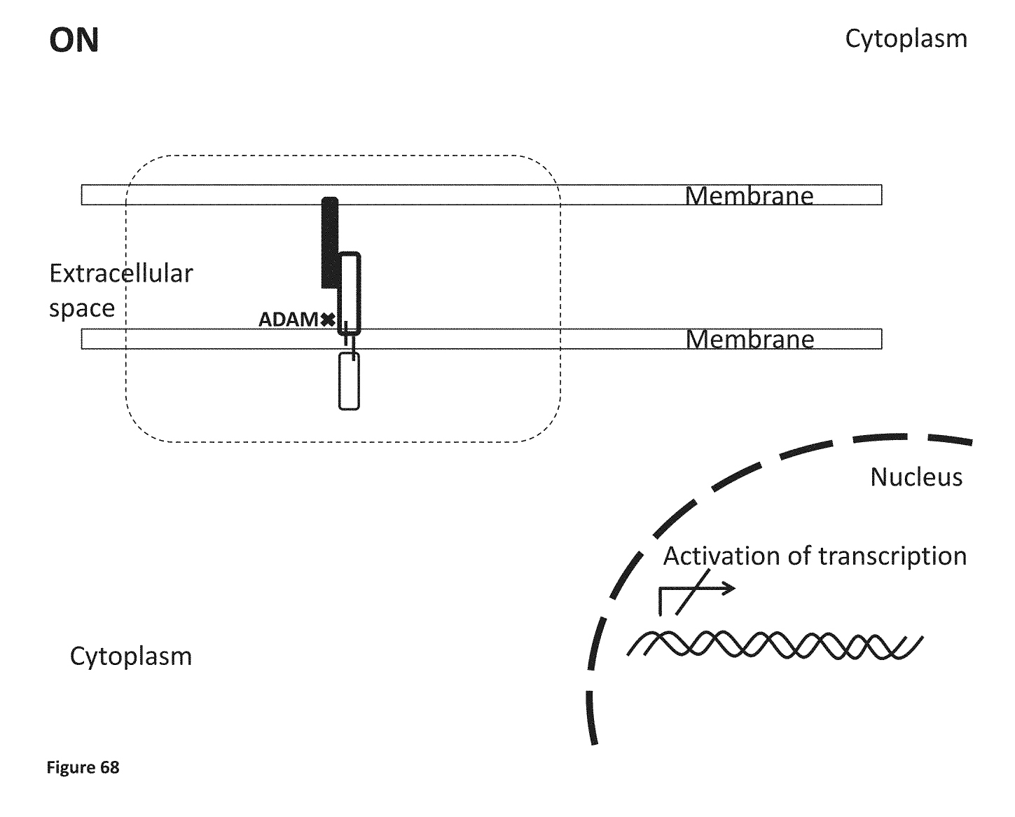

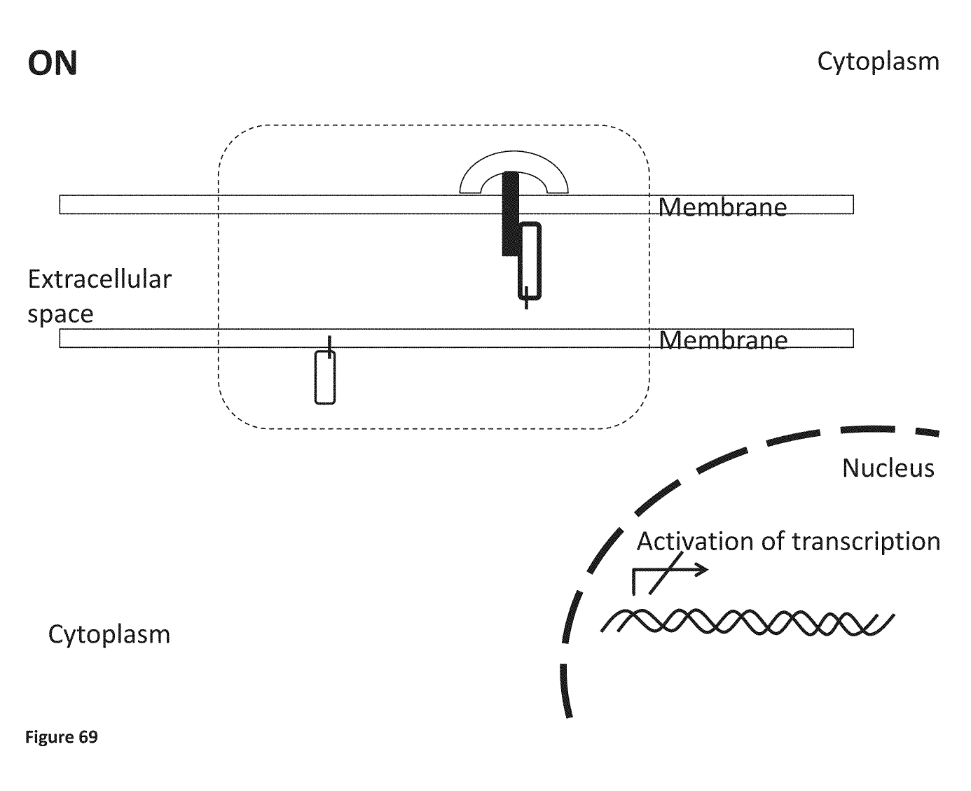

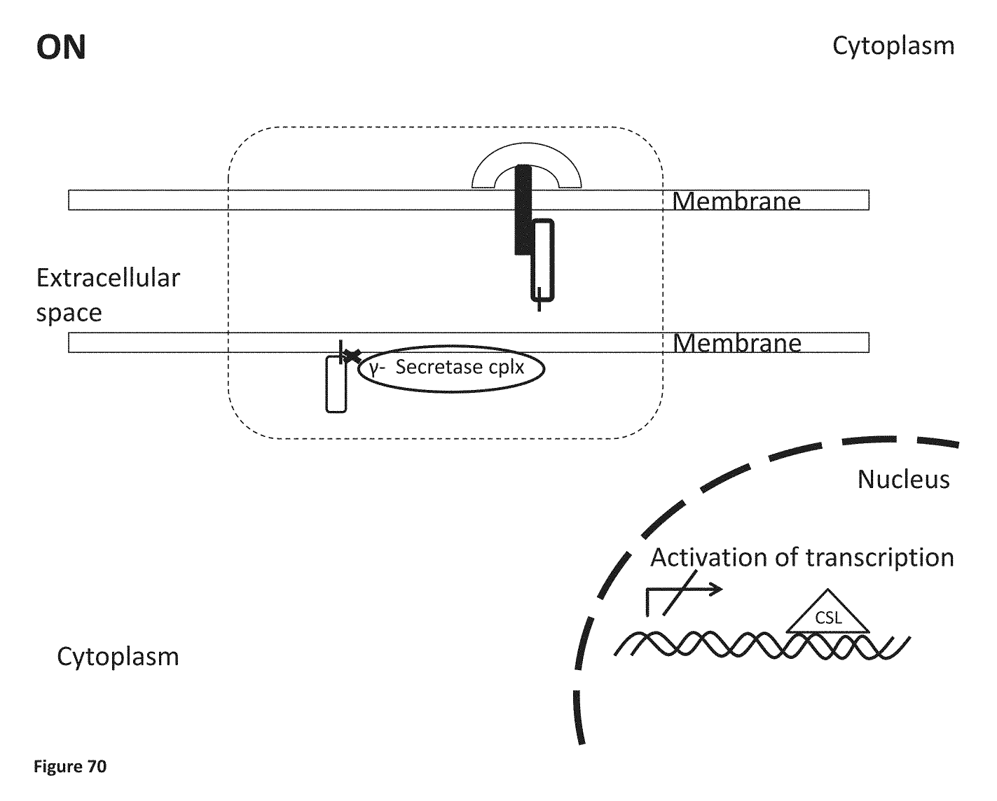

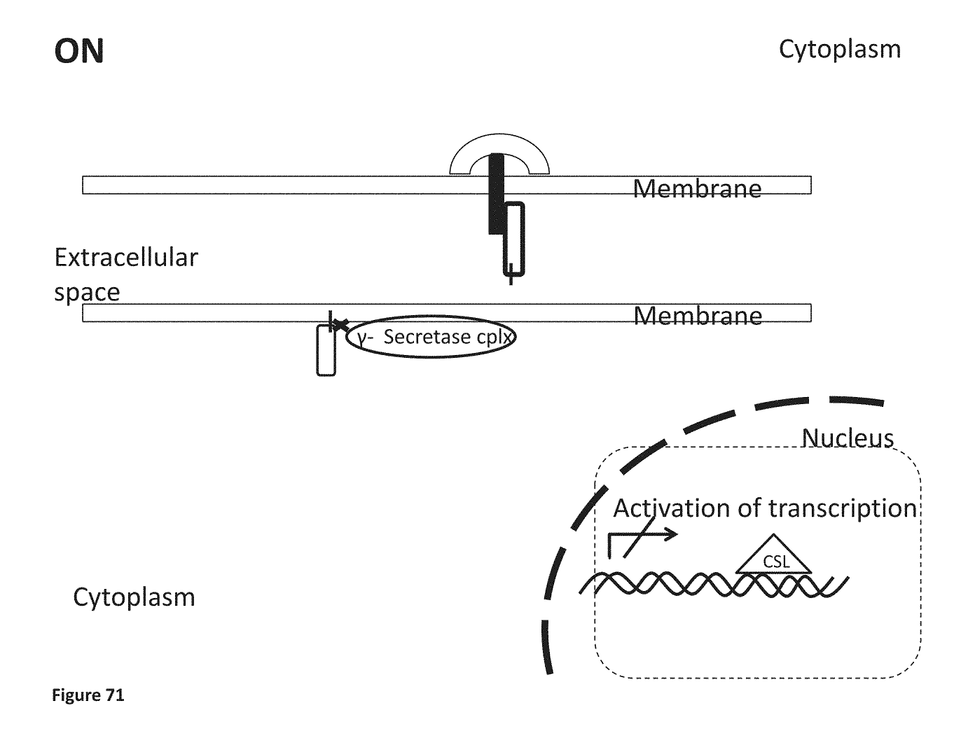

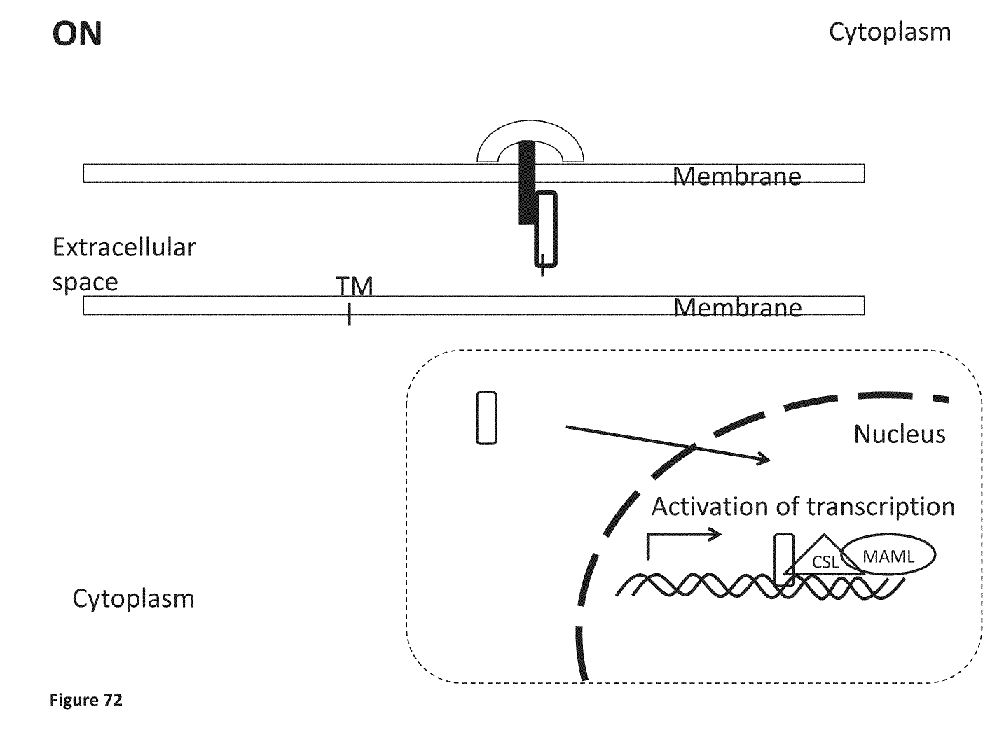

FIG. 65 through FIG. 72 and the following table provide example sequential step-by-step breakdown of an example contemporary understanding of the Notch pathway. Each step, represented by a table row and/or one of these figures, represents an example reaction step and is an example candidate for a replica reaction in the context of present invention and extensions of the present invention. As described earlier, the replica reactions can be implemented by embodiments of the present invention or extensions of the present invention in isolation (“stepwise”), and groups of contiguous steps can be implemented by embodiments of the present invention or extensions of the present invention together in a block of “one-pot” reactions (“blockwise”), the block representing a multiple-step segment or uninterrupted subset of the pathway. Example “stoichiometric” steps suitable for execution in the present invention are called out in the 4th column of the table. The 6th column of the table identifies example correspondences between the example contemporary understanding of the Notch pathway represented by the figures and the example steps of represented in of the table.

| | | Type of | Classes of | | Associated |
|---|---|---|---|---|---|
| Step | Description | Event | events | Constituents | FIGS. |
| 1 | Notch receptors are | Cleavage | Intracellular | Notch receptors, | |
| | processed in the ER and | Glycosylation | Transport | ER, Golgi | |
| | Golgi through cleavage and | and proteolysis | Stoichiometric | | |
| | glycosylation. | by furin-like | | | |
| | | Convertase | | | |
| 2 | The processed receptors in | Stabilized | Intra-membrane | Notch receptors | FIG. 66 |
| | the ER & Golgi causes a | Heterodimer | Stoichiometric | | |
| | Ca2+ stabilized heterodimer. | | | | |
| 3 | Notch receptor is then | Transportation | Membrane | Notch, Deltex, | FIG. 66, |
| | transported to the plasma | Regulation | Transduction | NUMB | FIG. 67 |
| | membrane to enable ligand | Inhibition | Extracellular | | |
| | binding regulated by Deltex | | Interaction | | |
| | and inhibited by NUMB. | | | | |
| 4 | Upon ligand binding, the | Proteolytic | Stoichiometric | NECD, ADAM | FIG. 68 |
| | NECD is cleaved away from | cleavage by | | | |
| | the TM-NICD domain by | ADAM, | | | |
| | ADAM. | removing | | | |
| | | NECD | | | |
| 5 | NECD remains bound to the | Endocytosis | Stoichiometric | NECD, ligand | FIG. 69 |
| | ligand and the complex goes | | Membrane | | |
| | through endocytosis within | | Transduction | | |
| | the signal sending cell. | | | | |
| 6 | In signaling receiving cell, γ- | Cleavage | Stoichiometric | γ-secretase, | FIG. 70 |
| | secretase releases NICD | | | NICD, | |
| | from the transmembrane. | | | | |
| 7 | Release of NICD from the | Translocation | Intracellular | NICD, CSL | FIG. 71 |
| | transmembrane allows | | Transport | | |
| | nuclear translocation where | | | | |
| | it associates with CSL | | | | |
| | transcription factor. | | | | |
| 8 | Association with CSL | Activation | Stoichiometric | CSL, MAML | FIG. 72 |
| | transcription factor activates | Transcription | Gene | | |
| | the canonical Notch target | | Expression | | |
| | genes: Myc, p21, & HES | | | | |
| | family members. | | | | |
| | (NICD, together with CSL | | | | |
| | and MAML, replaces the co- | | | | |
| | repressor and forms a | | | | |
| | transcription complex on | | | | |
| | Hes-1, Hes-5.) | | | | |

The 5th column of the above table lists constituents of the pathway step described in that row of the table. These constituents would be used in a replica reaction executed by the present invention corresponding to the pathway step described in the associated row of the above table.

The named constituents involved the example contemporary understanding of the Notch pathway represented by the above table can be produced in a laboratory or in some cases are commercially available or can be produced commercially in the future for use in the present invention and extensions of the present invention. As an example, the table below lists example sources for some of the constituents identified in the table above.

| Constituents | Description |
|---|---|
| Notch | http://www.rndsystems.com/product_results.aspx?m=1913 |
| | (Visited on Sep. 10, 2014) |
| Deltex | http://www.cusabio.com/protein-Recombinant_Protein-888692/ |
| | http://www.origene.com/protein/TP307944/DTX3L.aspx |
| | (Visited on Sep. 10, 2014) |
| NUMB | http://www.novusbio.com/NUMB-Recombinant-Protein_H00008650-P01.html |
| | (Visited on Sep. 10, 2014) |
| ADAM | http://www.mybiosource.com/datasheet.php?products_id=343002 |
| | (Visited on Sep. 12, 2014) |

### CLOSING REMARKS

The terms “certain embodiments”, “an embodiment”, “embodiment”, “embodiments”, “the embodiment”, “the embodiments”, “one or more embodiments”, “some embodiments”, and “one embodiment” mean one or more (but not all) embodiments unless expressly specified otherwise. The terms “including”, “comprising”, “having” and variations thereof mean “including but not limited to”, unless expressly specified otherwise. The enumerated listing of items does not imply that any or all of the items are mutually exclusive, unless expressly specified otherwise. The terms “a”, “an” and “the” mean “one or more”, unless expressly specified otherwise.

The foregoing description, for purpose of explanation, has been described with reference to specific embodiments. However, the illustrative discussions above are not intended to be exhaustive or to limit the present application to the precise forms disclosed. Many modifications and variations are possible in view of the above teachings. The embodiments were chosen and described in order to best explain the principles of the present application and its practical applications, to thereby enable others skilled in the art to best utilize embodiments of the present application and various embodiments with various modifications as are suited to the particular use contemplated.

While aspects of the present application has been described in detail with reference to disclosed embodiments, various modifications within the scope of the present application will be apparent to those of ordinary skill in this technological field. It is to be appreciated that features described with respect to one embodiment typically can be applied to other embodiments.

The present application can be embodied in other specific forms without departing from the spirit or essential characteristics thereof. The present embodiments are therefore to be considered in all respects as illustrative and not restrictive, the scope of the present application being indicated by the appended claims rather than by the foregoing description, and all changes which come within the meaning and range of equivalency of the claims are therefore intended to be embraced therein.

Although exemplary embodiments have been provided in detail, various changes, substitutions and alternations could be made thereto without departing from spirit and scope of the disclosed subject matter as defined by the appended claims. Variations described for the embodiments may be realized in any combination desirable for each particular application. Thus particular limitations and embodiment enhancements described herein, which may have particular advantages to a particular application, need not be used for all applications. Also, not all limitations need be implemented in methods, systems, and apparatuses including one or more concepts described with relation to the provided embodiments. Therefore, the present application properly is to be construed with reference to the claims.

What is claimed:

1. A method for implementing a “breadboard” approach for the study of a biochemical pathway, the method comprising:
separating a naturally occurring multiple-stage biochemical process into a plurality of smaller portions, at least one of which can to a degree of approximation be accurately emulated with a fluidic implementation replica;
implementing a fluidic implementation replica of at least one of the smaller portions, the replica comprising inputs for reactants, a reaction environment, and provisions for measurement;
providing the controlled introduction of a plurality of biochemical materials into the replica, and
making at least one consuming measurement relating to a resulting biochemical reaction within the replica,
wherein the biochemical reaction are controlled by a computer executing an algorithm, and
wherein the fluidic implementation replica emulates the at least one the smaller portions of the biochemical pathway.

2. The method of claim 1 wherein the replica is configured to provide outlets for removing the reaction products.

3. The method of claim 1 wherein the reaction environment is configured to comprise a membrane.

4. The method of claim 1 wherein the replica configured to comprise at least one sensor.

5. The method of claim 4 wherein the sensor comprises an antibody.

6. The method of claim 1 wherein the replica configured for the measurement is made with an external sensor.

7. The method of claim 1 wherein the replica configured for the measurement is made with an external instrument.

8. The method of claim 1 wherein the replica configured to provide molecular crowding.

9. The method of claim 1 wherein the replica configured to provide at least one confined reaction environment.

10. The method of claim 1 wherein the replica configured to provide at least one constrained reaction environment.

11. The method of claim 1 wherein a second fluidic implementation replica is used to emulate another smaller portion.

12. The method of claim 1 wherein the replica configured to provide the introduction of a competitive species.

13. The method of claim 1 wherein the replica configured to provide the introduction of a drug.

14. The method of claim 1 wherein the replica configured to provide the introduction of an environmental influence.

15. The method of claim 1 wherein the algorithm comprises a mathematical model.

16. The method of claim 14 wherein the algorithm models a gene transcription process.

17. The method of claim 1 wherein the computer also is executing a mathematical model in communication with the algorithm.

18. The method of claim 1 wherein the naturally-occurring signaling pathway is not entirely understood.

19. The method of claim 1 wherein the consuming measurement involves an antibody.

20. The method of claim 1 wherein the consuming measurement involves a fluorophore.
